(19) United States
(12) Patent Application Publication
(10) Pub. No.: US 2008/0255186 A1
(43) Pub. Date: Oct. 16, 2008

(54) Pyrazolo[3,4-B]Pyridine Compound, and Its Use as a Pde4 Inhibitor

(76) Inventors: Siegfried Benjamin Christensen, Collegeville, PA (US); Stuart Holman, Hertfordshire (GB); Suzanne Elaine Keeling, Hertfordshire (GB); Amyn Pyarali Sayani, Mississauga (CA)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/067,994

(22) PCT Filed: Sep. 29, 2006
(86) PCT No.: PCT/GB2006/003627

§ 371 Date: Mar. 25, 2008

Related U.S. Application Data
(60) Provisional application No. 60/721,597, filed on Sep. 29, 2005.

(51) Int. Cl. A61K 31/437; C07D 471/04; A61P 17/00

(52) U.S. Cl. 514/303; 546/119

(57) ABSTRACT

The invention provides N-{[1,6-diethyl-4-(tetrahydro-2H-pyran-4-ylamino)-1H-pyrazolo[3,4-b]pyridin-5-yl]methyl}-3-methyl-5-isoxazolecarboxamide

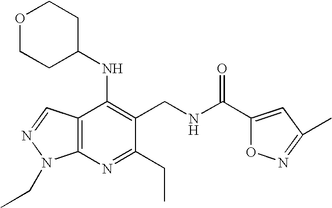

or a salt thereof, such as the compound or a pharmaceutically acceptable salt thereof. The invention also provides the use of the compound or salt as an inhibitor of phosphodiesterase type IV (PDE4.

The present invention relates to a pyrazolo[3,4-b]pyridine compound or a salt thereof, processes for their preparation, intermediates usable in these processes, and pharmaceutical compositions containing the compound or a salt thereof. The invention also relates to the use of the pyrazolo[3,4-b]pyridine compound or a salt thereof in therapy, for example as inhibitors of phosphodiesterase type IV (PDE4) and/or for the treatment and/or prophylaxis of inflammatory and/or allergic diseases such as atopic dermatitis.

### BACKGROUND TO THE INVENTION

WO 2004/024728 A2 (PCT/EP2003/011814, filed on 12 Sep. 2003, published on 25 Mar. 2004, Glaxo Group Limited), and incorporated herein by reference in its entirety as though fully set forth, discloses pyrazolo[3,4-b]pyridine compounds or salts thereof with a 4-NHR³ group and a 5-C(O)—X group (wherein X is NR⁴R⁵ or OR^{5a}), according to the following formula (I):

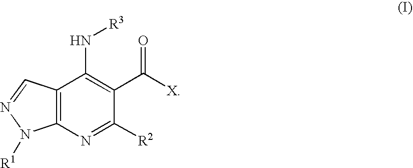

In WO 2004/024728 A2, the pyrazolo[3,4-b]pyridine compounds of formula (I) and salts thereof disclosed therein are disclosed as being inhibitors of phosphodiesterase type IV (PDE4).

WO 2004/056823 A1 (PCT/EP2003/014867, filed on 19 Dec. 2003, published on 8 Jul. 2004, Glaxo Group Limited), and incorporated herein by reference in its entirety as though fully set forth, discloses and claims further pyrazolo[3,4-b]pyridine compounds or salts thereof. WO 2004/056823 A1 also discloses the use of these compounds as PDE4 inhibitors.

WO 2004/024728 has been reviewed, and WO 2004/056823 mentioned, in Expert Opin. Ther. Patents, 2005 (January edition), 15(1), 111-114.

WO 2005/058892 A1 (PCT/EP2004/014490, filed on 17 Dec. 2004, published on 30 Jun. 2005, Glaxo Group Limited), and incorporated herein by reference in its entirety as though fully set forth, discloses further pyrazolo[3,4-b]pyridine compounds or salts thereof and their use as PDE4 inhibitors.

Further pyrazolo[3,4-b]pyridine compounds or salts thereof, and their use as PDE4 inhibitors, are disclosed in copending patent publications WO 2005/090348 A1 (PCT/GB2005/000983), WO 2005/090354 A1 (PCT/GB2005/000987), WO 2005/090352 A1 (PCT/EP2005/003038), and WO 2005/090353 A1 (PCT/GB2005/000976) (all Glaxo Group Limited, all PCT-filed on 15 Mar. 2005 and all published on 29 Sep. 2005).

### The Invention

We have now found a new pyrazolo[3,4-b]pyridine compound which inhibits phosphodiesterase type IV (PDE4), and which for example appears to be suitable for inclusion in an external-topical pharmaceutical composition and/or which appears to be usable by external topical administration (e.g. to a mammal such as a human or pig), in particular by topical administration to the skin.

The present invention therefore provides N-{[1,6-diethyl-4-(tetrahydro-2H-pyran-4-ylamino)-1H-pyrazolo[3,4-b]pyridin-5-yl]methyl}-3-methyl-5-isoxazolecarboxamide

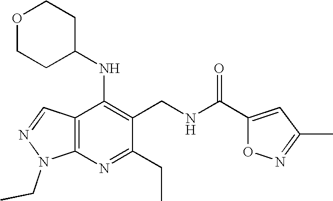

or a salt thereof.

In particular, the present invention provides N-{[1,6-diethyl-4-(tetrahydro-2H-pyran-4-ylamino)-1H-pyrazolo[3,4-b]pyridin-5-yl]methyl}-3-methyl-5-isoxazolecarboxamide or a pharmaceutically acceptable salt thereof.

In particular, the present invention provides N-{[1,6-diethyl-4-(tetrahydro-2H-pyran-4-ylamino)-1H-pyrazolo[3,4-b]pyridin-5-yl]methyl}-3-methyl-5-isoxazolecarboxamide. This is the so-called “free base” form, also called “the compound of the invention” or similar.

The above-defined compound or salt of the present invention (hereinafter “the/a compound or salt of the invention” or “the compound of the invention or the salt thereof” or similar) is a compound of formula (I) or a salt thereof (in particular, the compound or a pharmaceutically acceptable salt thereof):

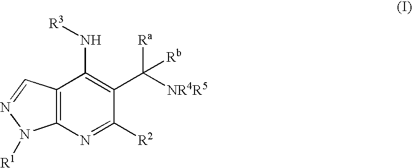

wherein:
R¹ is ethyl;
R² is ethyl;
R³ is a heterocyclic group of sub-formula (bb) which is not substituted on a ring carbon:

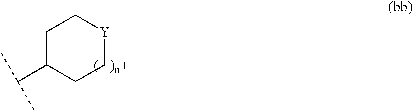

in which n¹ is 1; and in which Y is O;
and wherein:
R^{a} is a hydrogen atom (H);
R^{b} is a hydrogen atom (H);
R⁴ is a hydrogen atom (H);
R⁵ is —C(O)—(CH₂)ₙ—Ar; wherein n is 0;
(that is: NR⁴R⁵ is —NH—C(O)—Ar)
and Ar has the sub-formula (z) which is sub-formula (z9):

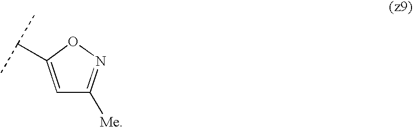

In compounds which may be usable in the preparation of the compounds of formula (I), an “alkyl” group or moiety may be straight-chain or branched. Alkyl groups, for example C₁ alkyl or C₁₋₆alkyl or C₁₋₄alkyl or C₁₋₃alkyl or C₁₋₂alkyl, which may be employed include C₁₋₆alkyl or C₁₋₄alkyl or C₁₋₃alkyl or C₁₋₂alkyl such as methyl, ethyl, n-propyl, n-butyl, n-pentyl, or n-hexyl or any branched isomers thereof such as isopropyl, t-butyl, sec-butyl, isobutyl, 3-methylbutan-2-yl, 2-ethylbutan-1-yl, or the like.

A corresponding meaning is intended for “alkoxy”, and like terms derived from alkyl. For example, “alkoxy” such as C₁₋₆alkoxy or C₁₋₄alkoxy or C₁₋₂alkoxy includes methoxy, ethoxy, propyloxy, and oxy derivatives of the alkyls listed above. “Alkylsulfonyl” such as C₁₋₄alkylsulfonyl includes methylsulfonyl (methanesulfonyl), ethylsulfonyl, and others derived from the alkyls listed above. “Alkylsulfonyloxy” such as C₁₋₄alkylsulfonyloxy includes methanesulfonyloxy (methylsulfonyloxy), ethanesulfonyloxy, et al.

“Fluoroalkyl” includes alkyl groups with one, two, three, four, five or more fluorine substituents, for example C₁₋₄fluoroalkyl or C₁₋₃fluoroalkyl or C₁₋₂fluoroalkyl such as monofluoromethyl, difluoromethyl, trifluoromethyl, pentafluoroethyl, 2,2,2-trifluoroethyl (CF₃CH₂—), 2,2-difluoroethyl (CHF₂CH₂—), 2-fluoroethyl (CH₂FCH₂—), etc. “Fluoroalkoxy” includes C₁₋₄fluoroalkoxy or C₁₋₂fluoroalkoxy such as trifluoromethoxy, pentafluoroethoxy, monofluoromethoxy, difluoromethoxy, etc. “Fluoroalkylsulfonyl” such as C₁₋₄fluoroalkylsulfonyl includes trifluoromethanesulfonyl, pentafluoroethylsulfonyl, etc.

A halogen atom (“halo”) present in compounds, for example in the compounds of formula (I), means a fluorine, chlorine, bromine or iodine atom (“fluoro”, “chloro”, “bromo” or “iodo”), for example fluoro, chloro or bromo.

In the compound of formula (I) or the salt thereof of the present invention, R³ is tetrahydro-2H-pyran-4-yl; that is NHR³ is of sub-formula (h) as shown below:

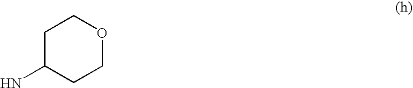

In the sub-formulae (h) above, the —NH— connection point of the NHR³ group to the 4-position of the pyrazolopyridine of formula (I) is underlined.

The compound of the invention or the salt thereof can suitably be for external topical administration (e.g. to a mammal such as a human or pig), in particular for topical administration to the skin.

The compound of the invention or a pharmaceutically acceptable salt thereof can suitably be contained/comprised in a pharmaceutical composition suitable and/or adapted for external topical administration (e.g. to a mammal such as a human or pig), in particular in a pharmaceutical composition suitable and/or adapted for topical administration to the skin (e.g. to a mammal such as a human or pig).

### Salts, Solvates, Isomers, Tautomeric Forms, Molecular Weights, etc.

Because of their potential use in medicine, the compound of formula (I) may be used in the form of a pharmaceutically acceptable salt thereof. Pharmaceutically acceptable salts can include acid addition salts.

For example, a pharmaceutically acceptable acid addition salt of the compound of the invention can be formed (or can have been formed) by mixing (e.g. intimately mixing) N-{[1,6-diethyl-4-(tetrahydro-2H-pyran-4-ylamino)-1H-pyrazolo[3,4-b]pyridin-5-yl]methyl}-3-methyl-5-isoxazolecarboxamide with a pharmaceutically acceptable acid having a pKa of 1.5 or less.

In one embodiment, a pharmaceutically acceptable acid addition salt is optionally formed by mixing (e.g. intimately mixing) a compound of formula (I) with a suitable pharmaceutically acceptable inorganic or organic acid (such as hydrobromic, hydrochloric, sulfuric, nitric, phosphoric, p-toluenesulfonic, benzenesulfonic, methanesulfonic, ethanesulfonic, or naphthalenesulfonic such as 2-naphthalenesulfonic acid), optionally in (e.g. dissolved in) a suitable solvent, and/or optionally in (e.g. dissolved in) an organic solvent such as methanol, ethanol, isopropanol, ethyl acetate, acetonitrile and/or dichloromethane, to give the salt, which is usually isolated for example by crystallisation and filtration. In one embodiment, the compound of formula (I) is for example dissolved in a suitable solvent, and/or in an organic solvent such as methanol, ethanol, isopropanol, ethyl acetate, acetonitrile and/or dichloromethane, and the pharmaceutically acceptable inorganic or organic acid (e.g. in solution or in isolated form) is then added, to prepare the pharmaceutically acceptable salt, which is usually isolated for example by crystallisation and filtration. For preparation of a hydrochloride salt, an anhydrous solution (e.g. 1-5M, e.g. 2M) of HCl in dioxane or diethyl ether, or aqueous hydrochloric acid, or HCl gas, is optionally used as the acid. For preparation of a sulfate or nitrate salt, in one embodiment an aqueous, methanolic or ethanolic solution of sulfuric acid or nitric acid respectively is optionally added to a solution of the compound of formula (I) in a water-miscible organic solvent.

A pharmaceutically acceptable acid addition salt of a compound of formula (I) can comprise or be for example a hydrobromide, hydrochloride, sulfate, nitrate, phosphate, p-toluenesulfonate, benzenesulfonate, methanesulfonate, ethanesulfonate, or naphthalenesulfonate (e.g. 2-naphthalenesulfonate) salt of the compound of formula (I).

Other non-pharmaceutically acceptable salts, e.g. oxalates or trifluoroacetates, may be used, for example in the isolation of compounds of the invention, and are included within the scope of this invention.

The invention includes within its scope all possible stoichiometric and non-stoichiometric forms of the salts of the compounds of formula (I).

Also included within the scope of the invention are all solvates, hydrates and polymorphs of compounds and salts of the invention.

Certain salts of the compound of the invention (or the tautomer of the amide moiety in the compound of the invention), included in the present invention, may be present as isomers, e.g. positional, geometric or optical isomers. The present invention includes within its scope all such isomers, including racemates, enantiomers and mixtures thereof.

Certain of the groups, e.g. heteroaromatic ring systems or amide moiety(ies), included in the compound of formula (I) or a salt thereof may exist in one or more tautomeric forms. The present invention includes within its scope all such tautomeric forms, including mixtures.

### Synthetic Process Routes

The following non-limiting processes can generally be used to prepare the compound of the invention:

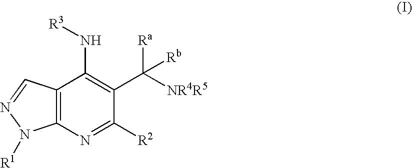

In the compound of Formula (I), R^{a} and R^{b} are both a hydrogen atom (H), R⁴ is H, R⁵ is —C(O)—(CH₂)ₙ—Ar; wherein n is 0 and wherein Ar is as defined herein, and R¹, R² and R³ are as defined herein.

### Process A

To prepare a compound of formula (I), an amine of formula (II) or a salt thereof can be reacted with a compound Ar—C(O)—X¹, wherein X¹ is a leaving group substitutable by the NH₂ amine moiety of the compound of formula (II):

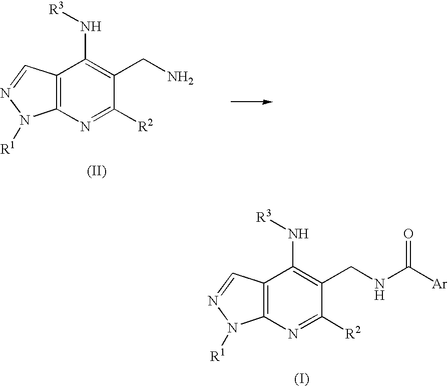

Ar—C(O)—X¹ is typically an activated derivative of carboxylic acid Ar—C(O)—OH.

In the reaction of (II) to (I), a compound Ar—C(O)—X¹ can for example be the acid chloride Ar—C(O)Cl (wherein X¹ is a chlorine atom (Cl)). For use of Ar—C(O)Cl, the reaction is typically carried out in the presence of a base such as diisopropylethylamine (ⁱPr₂NEt=DIPEA) and/or in a suitable non-aqueous non-alcohol organic solvent (e.g. anhydrous solvent) such as acetonitrile (e.g. anhydrous) or DMF, for example at room temperature (e.g. about 18 to about 25° C.).

The acid chloride Ar—C(O)Cl can typically be formed from the carboxylic acid (a) by reaction with thionyl chloride, either in a suitable organic solvent such as chloroform or without solvent, or (b) by reaction with oxalyl chloride (optionally also with e.g. diethyl formamide or DMF), for example in a suitable organic solvent such as dichloromethane.

Alternatively, the compound Ar—C(O)—X¹ can be an activated derivative of the carboxylic acid Ar—C(O)—OH wherein the leaving group X¹ is

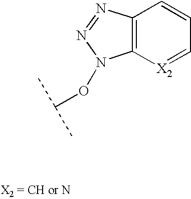

The activated compound Ar—C(O)—X¹, where X¹ is as shown above, can optionally be formed from the carboxylic acid Ar—C(O)—OH by the following reaction (a). In reaction (a), the carboxylic acid Ar—C(O)—OH is reacted with (i), (ii), (iii) or (iv):

(i) O-(7-azabenzotriazol-1-yl)-N,N,N′,N′-tetramethyluronium hexafluorophosphate (HATU) (when X₂ is N), or
(ii) O-(benzotriazol-1-yl)-N,N,N′,N′-tetramethyluronium hexafluorophosphate (HBTU) (when X₂ is CH), or
(iii) 2-(1H-benzotriazol-1-yl)-1,1,3,3-tetramethyluronium tetrafluoroborate (TBTU) (when X₂ is CH), or
(iv) benzotriazol-1-yl-oxy-trispyrrolidinophosphonium hexafluorophosphate (PyBOP).

In one embodiment of reaction (a), reaction (a) is e.g. carried out in the presence of a tertiary amine base such as diisopropylethylamine (ⁱPr₂NEt=DIPEA), and/or in the presence of a non-aqueous non-alcohol organic solvent (e.g. anhydrous solvent) such as dimethyl formamide (DMF, e.g. anhydrous DMF) or acetonitrile e.g. anhydrous acetonitrile. In one embodiment, reaction (a) is e.g. carried out at room temperature (e.g. about 18 to about 25° C.), and/or for a reaction time of from 2 to 48 hours or 6 to 48 hours such as 12 to 24 hours, for example about 15-18 hours. For example, reaction (a) can optionally be carried out under anhydrous conditions.

In an alternative embodiment, the activated compound Ar—C(O)—X¹ is for example formed from the carboxylic acid Ar—C(O)—OH by the following reaction (b). In optional reaction (b), the carboxylic acid Ar—C(O)—OH is reacted with a suitable organic di-substituted carbodiimide (R^{C1}N═C═NR^{C2} wherein R^{C1} and R^{C2} are independent organic groups such as, independently, C₁₋₄alkyl (e.g. ethyl), cyclohexyl or 3-dimethylaminopropyl), such as 1-(3-dimethylaminopropyl)-3-ethylcarbodiimide or a salt thereof (EDC) e.g. the hydrochloride salt, or such as dicyclohexylcarbodiimide (DCC), and the resulting carbodiimide-acid adduct Ar—C(O)—X¹ wherein X¹ is O—C(NHR^{C1})═NR^{C2} for example then reacts with the amine of formula (II) or a salt thereof.

In one embodiment, this reaction (b) is for example carried out in the presence of a non-aqueous non-alcohol organic solvent (e.g. anhydrous solvent) such as dimethyl formamide (DMF) or acetonitrile and/or e.g. at room temperature and/or e.g. under anhydrous conditions. In one embodiment, the reaction is carried out in the presence of a tertiary amine base such as diisopropylethylamine (iPr₂NEt=DIPEA).

In one alternative to reaction (b), the reaction of Ar—C(O)—OH with the carbodiimide is optionally carried out in the presence of 1-hydroxybenzotriazole (HOBT), in which case, an activated compound Ar—C(O)—X¹ wherein X¹ is

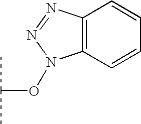

is often the compound which reacts with the amine of formula (II) or a salt thereof (cf. reaction (a) above).

The amine compound of formula (II) can be prepared by hydrogenation of an azide compound of formula (IIIa):

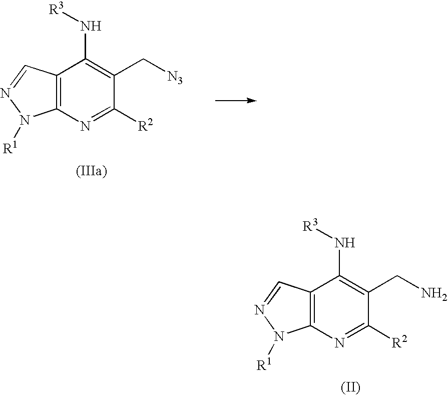

Hydrogenation conditions can for example include H₂/palladium on carbon (e.g. 5 to 10% palladium on carbon), for example with ethanol, methanol, isopropyl alcohol or ethyl acetate (in particular ethanol) solvent.

The azide compound of formula (IIIa) can generally be prepared by reaction of a compound of formula (IIIb), wherein X^{3b} is a leaving group displaceable by azide, with a metal azide such as sodium azide, lithium azide or potassium azide:

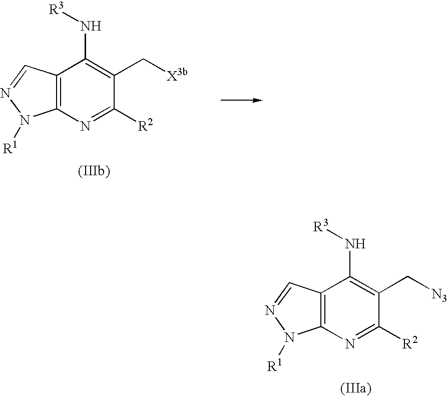

Typical conditions for the (IIIb) to (IIIa) reaction, e.g. with sodium azide or lithium azide, can e.g. include DMSO solvent (e.g. dry) or DMF solvent (e.g. dry) and/or reaction at room temperature. See for example Reference Intermediate 7 and Intermediate 8. In one embodiment, X^{3b} is a chlorine atom (Cl) or an organic sulfonate such as methanesulfonate, trifluoromethanesulfonate CF₃SO₂— or p-toluenesulfonate, in particular a chlorine atom.

Compounds of formula (IIIb), in particular wherein X^{3b} is Cl or an organic sulfonate, can generally or sometimes be prepared by conversion (e.g. chlorination) of an alcohol compound of formula (IIIc). This can for example be by reaction with SOCl₂ (thionyl chloride) for chlorination (when X^{3b} is Cl), or by reaction with methanesulfonyl chloride (when X^{3b} is methanesulfonate) or p-toluenesulfonyl chloride (when X^{3b} is p-toluenesulfonate). The chlorination reaction with thionyl chloride may require heating, e.g. to about 60 to about 90° C., for example at about 85° C.:

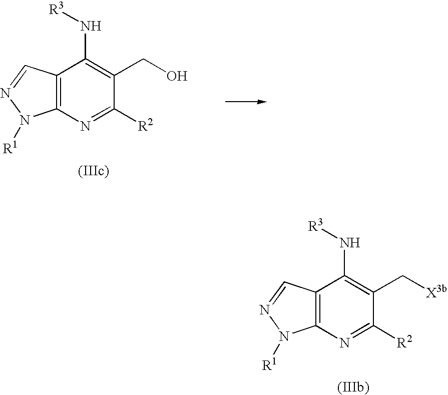

In one alternative method, azide compounds of formula (IIIa) may be preparable directly from the alcohol compound of formula (IIIc). For example, reacting compounds of formula (IIIc) with an azide, e.g. sodium azide, in the presence of carbon tetrabromide and triphenylphosphine may give a compound of formula (IIIa) (e.g see Toyota et. al. Journal of Organic Chemistry, 2000, 65(21), 7110-7113):

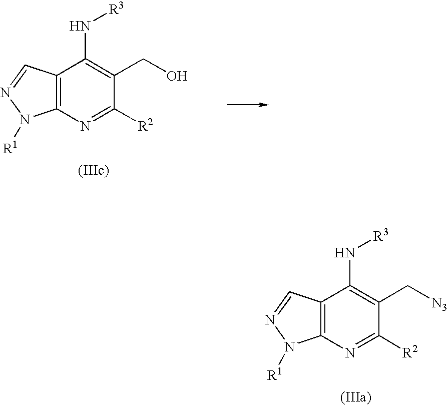

In another alternative process which is of particular interest, the amine compound of formula (II) or a salt thereof can be prepared directly from the compound of formula (IIIb) or a salt thereof (for example wherein X^{3b} is a chlorine atom, for example from the benzenesulfonate salt of the compound of formula (IIIb) e.g. wherein X^{3b} is a chlorine atom), without converting (IIIb) to the azide compound of formula (IIIa):

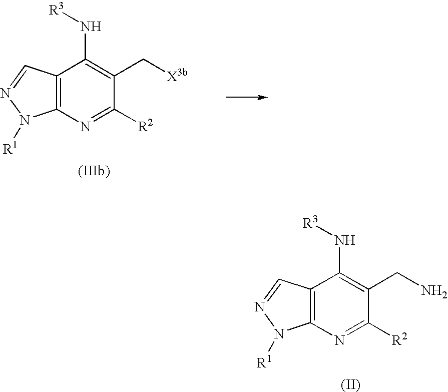

For example, this reaction of (IIIb) or a salt thereof to the amine (II) or a salt thereof can generally be carried out by reaction of the compound of formula (IIIb) (for example wherein X^{3b} is or comprises a chlorine atom) with an aminating agent such as lithium hexamethyldisilazide, sodium hexamethyldisilazide or potassium hexamethyldisilazide (in particular lithium hexamethyldisilazide, e.g. with slow mixing/addition), in a suitable non-aqueous non-alcohol organic solvent (e.g. anhydrous solvent) such as tetrahydrofuran, for example at a suitable temperature of, for example, about 25 to about 50° C., e.g. ca. 30-45° C. or ca. 35-40° C. The reaction is suitably followed by treatment with an aqueous acid such as aqueous hydrochloric acid (e.g. 2-10M, e.g. about 5M), e.g. at a suitable temperature such as from 0° C. to room temperature, for example at about 10° C.

In a simplified process, wherein the compound of formula (IIIb) has X^{3b} as a chlorine atom, in one suitable embodiment the alcohol compound of formula (IIIc) or a salt thereof is converted into the amine of formula (II) or a salt thereof, without substantially purifying and/or without substantially isolating the compound of formula (IIIb) or the salt thereof wherein X^{3b} is a chlorine atom. In this embodiment, the compound of formula (IIIb) or a salt thereof wherein X^{3b} is a chlorine atom can for example be in the form of the benzenesulfonate salt:

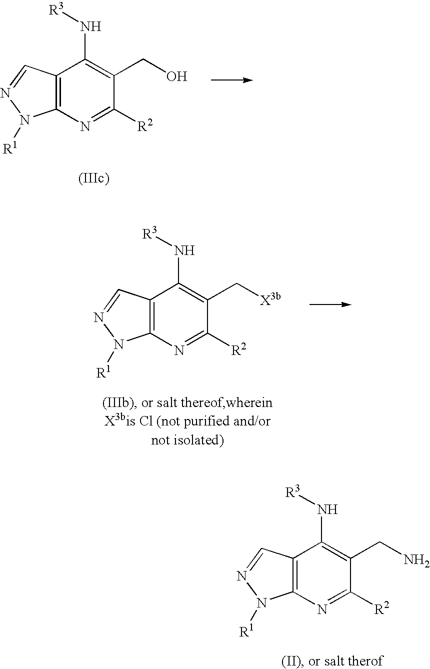

The alcohol compound of formula (IIIc) can be prepared by reduction of an ester compound of formula (IV), wherein R^{e} is C₁₋₄ straight-chain alkyl such as ethyl, in the presence of a reducing agent. The reducing agent can for example be diisobutylaluminium hydride or lithium borohydride (LiBH₄). For diisobutylaluminium hydride reduction, the reduction can e.g. be in dichloromethane and/or toluene solvent (e.g. see Intermediate 4 or Reference Intermediate 3 herein) and/or can e.g. be carried out at about 0° C. and/or under nitrogen and/or with careful temperature control and/or with gradual addition or mixing of diisobutylaluminium hydride. For lithium borohydride (LiBH₄), see for example Intermediate 4, Alternative Synthesis B herein:

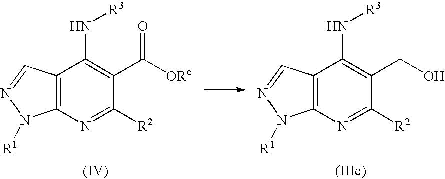

Compounds of formula (IV) can generally be prepared according to a method, for example as described by Yu et. al. in J. Med Chem., 2001, 44, 1025-1027, by reaction of a compound of formula (V) with an amine of formula R³NH₂ or a salt thereof such as a hydrochloride salt thereof. The reaction is for example carried out in the presence of a tertiary amine base such as triethylamine or N,N-diisopropylethylamine, and/or in an organic solvent such as 1-methyl-2-pyrrolidinone (NMP), ethanol, dioxane or acetonitrile. The reaction may comprise heating e.g. heating to ca. 60-180° C. or 80-180° C., for example ca. 60-100° C. (e.g. ca. 80-90° C.) or ca. 110-160° C., for example depending on the reflux temperature or boiling point of the solvent(s) used. For an example process, see e.g. Intermediate 2 herein, or see e.g. the process shown in Yu et. al. in J. Med. Chem., 2001, 44, 1025-1027 (see steps c+d of Scheme 1 therein):

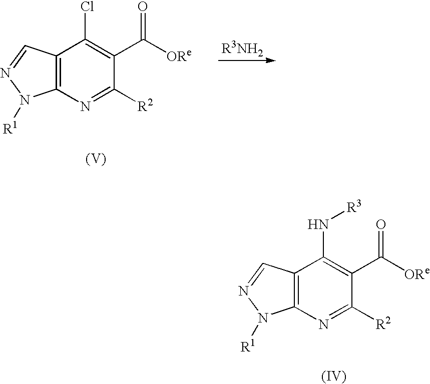

The amine of formula R³NH₂ is commercially available, e.g. from Combi-Blocks Inc. and/or from Peakdale Molecular Ltd. See Intermediate 12 one possible synthesis of the hydrochloride salt of R³NH₂.

In one embodiment, a compound of formula (V) is for example prepared by reaction of a compound of formula (VI) with (R²)(Cl)C═C(CO₂R^{e})₂ (a compound of formula (XIV)), followed by reaction with phosphorous oxychloride (POCl₃). The compound (R²)(Cl)C═C(CO₂R^{e})₂ can for example be diethyl (1-chloropropylidene)propanedioate (wherein R² is Et and R^{e} is Et):

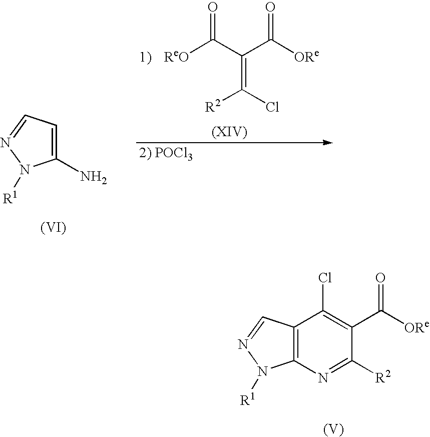

In one embodiment of the (VI) to (V) process, the compound of formula (VI) is reacted with a dialkyl (1-chloroalkylidene)propanedioate with heating, for example in a suitable organic solvent such as toluene, and for example in the presence of a suitable base such as triethylamine, e.g. at a suitable temperature such as the reflux temperature of the solvent. The conditions for the reaction of the intermediate [formed from (VI) and the dialkyl (1-chloroalkylidene)propanedioate] with phosphorous oxychloride (POCl₃) can include heating, e.g. heating at the reflux temperature of phosphorous oxychloride.

For an example of the compound (VI) to compound (V) process, see for example the Intermediate 1 synthesis hereinafter wherein R²=ethyl and R¹=ethyl and R^{e} is ethyl.

In one embodiment, a compound (R²)(Cl)C═C(CO₂R^{e})₂ (a compound of formula (XIV)) is prepared by reaction of a compound of formula (XV), with phosphorus oxychloride (POCl₃) in the presence of a suitable base such as tributylamine, at a suitable temperature such as ca. 80-130° C., for example ca. 100-120° C.:

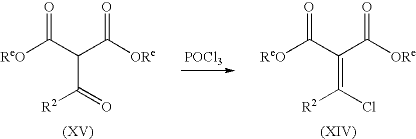

In one embodiment, a compound of formula (XV) is prepared by reaction of a dialkyl malonate of formula (XVI) with magnesium chloride and a suitable non-aqueous base such as triethylamine, in a suitable solvent (e.g. anhydrous solvent) such as acetonitrile, at a suitable temperature such as ca. 5-10° C., followed by addition of an acid chloride of formula (XVII) (propanoyl chloride when R² is ethyl), at a suitable temperature such as between 10° C. and room temperature. In one embodiment, the reaction is for example carried out under anhydrous conditions:

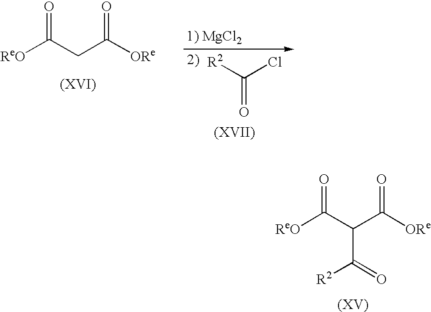

The compounds of formulae (XVI) and (XVII) where R^{e} and R² respectively represent ethyl are commercially available, e.g. from Aldrich or elsewhere.

The desired amino pyrazole of formula (VI), which (for R¹ is ethyl) is 1-ethyl-1H-pyrazol-5-amine, is commercially available, e.g. is commercially available from one or more of the following suppliers: Aldrich Chemical Company Inc., Albemarle Corporation, Art-Chem GmbH, Enamine and/or TimTec Corporation. However, alternatively, the amino pyrazole (VI) is optionally prepared, for example, by reaction of cyanoethyl hydrazine with a suitable aldehyde of formula R⁴⁰CHO in a solvent such as ethanol, with heating, followed by reduction, for example reduction with sodium in a suitable solvent such as t-butanol. (See for example the method(s) described by Dorgan et. al. in J. Chem. Soc., Perkin Trans. 1, (4), 938-42; 1980.) R⁴⁰ should be chosen so as to contain one less carbon atom than R¹, so that for example R⁴⁰=methyl will afford R¹=ethyl.

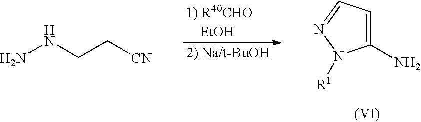

In an alternative to the reaction of compound (V) to prepare a 4-amino 5-ester pyrazolopyridine compound of Formula (IV), instead of the 4-chloro substituent in the compound of formula (V), it is likely that a different non-fluorine halogen atom such as a bromine atom, or another suitable leaving group which is displaceable by an amine of formula R³NH₂, can sometimes be used at the 4-position of the pyrazolopyridine. In one embodiment, the leaving group displaceable by the amine is for example R^{LA}, in the compound of formula (Va) shown below, wherein R^{LA} is a bromine atom or an alkoxy group OR³⁵ such as OC₁₋₄alkyl (in particular OEt) or a group —O—S(O)₂—R³⁷. Here, R³⁷ can e.g. be C₁₋₂alkyl such as methyl, C₁fluoroalkyl such as CF₃, phenyl, or 4-methyl-phenyl. The reaction of the compound of formula (Va) with the amine of formula R³NH₂ or a salt thereof is optionally carried out with or without solvent, and e.g. may comprise heating:

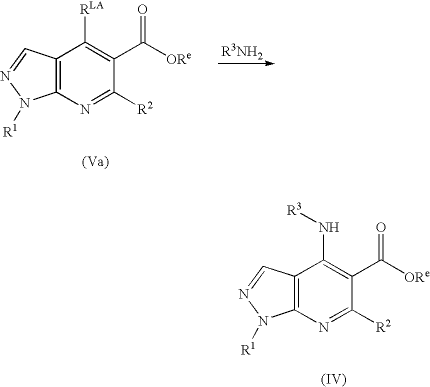

Compounds (Va) wherein R^{LA} is the group —O—S(O)₂—R³⁷ are likely to be preparable by reaction of the corresponding 4-hydroxy-pyrazolopyridine with R³⁷—S(O)₂—Cl such as CF₃—S(O)₂—Cl. The compound of formula (Va) wherein R^{LA} is OEt and R^{e} is Et is likely to be preparable, e.g. via the following reaction from the 4-chloro-pyrazolopyridine (V):

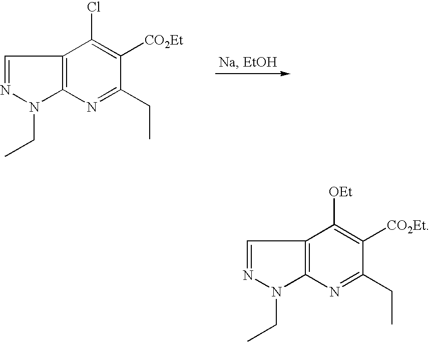

### Process Summary

The present invention therefore also provides a method of (process for) preparing a compound of formula (I) or a salt (e.g. pharmaceutically acceptable salt) thereof:

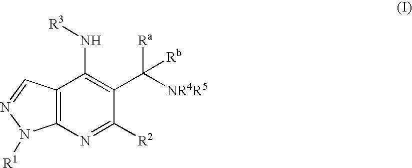

using the final step of Process A, generally as described hereinabove,
and optionally converting the compound of formula (I) into a salt thereof e.g. a pharmaceutically acceptable salt thereof.

Therefore, another aspect of the present invention provides a process for preparing a compound of formula (I) or a salt (e.g. pharmaceutically acceptable salt) thereof:

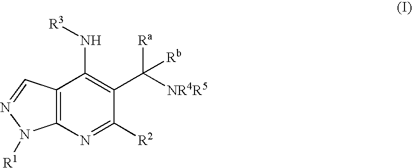

wherein R¹, R², R³, R⁴, R⁵, R^{a} and R^{b} are as defined herein, which process comprises:
(A) reacting an amine of formula (II) or a salt thereof

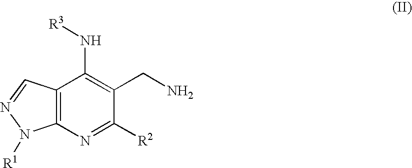

with a compound Ar—C(O)—X¹, wherein X¹ is a leaving group substitutable by the NH₂ amine moiety of the compound of formula (II);
and optionally converting the compound of formula (I) into a salt thereof e.g. a pharmaceutically acceptable salt thereof.

In this process aspect of the invention, preferred, suitable or optional features of process (A), can be as described above for Process A (e.g. those described above as preferred, suitable, optional or example features of this process), with all necessary changes being made.

In process (A), In the reaction of (II) to (I), a compound Ar—C(O)—X¹ can for example be the acid chloride Ar—C(O)Cl (i.e. X¹ is a chlorine atom (Cl)). Alternatively, the compound Ar—C(O)—X¹ can be an activated derivative of the carboxylic acid Ar—C(O)—OH wherein the leaving group X¹ is

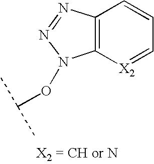

Alternatively, in the compound Ar—C(O)—X¹, X¹ can be O—C(NHR^{C1})═NR^{C2} wherein R^{C1} and R^{C2} are independent organic groups such as, independently, C₁₋₄alkyl (e.g. ethyl), cyclohexyl or 3-dimethylaminopropyl. In one embodiment, R^{C1} and R^{C2} can e.g. both be cyclohexyl; or one of R^{C1} and R^{C2} is ethyl and the other of R^{C1} and R^{C2} is 3-dimethylaminopropyl.

The present invention also provides: (B) a process for preparing a pharmaceutically acceptable salt of a compound of formula (I) comprising conversion of the compound of formula (I) or a salt thereof into a or the desired pharmaceutically acceptable salt of the compound of formula (I). Such processes can for example be as described herein, e.g. as described in the Salts section hereinabove. For example, a pharmaceutically acceptable salt can be an acid addition salt. In one embodiment, a pharmaceutically acceptable acid addition salt is optionally prepared by reaction of a compound of formula (I) with a suitable inorganic or organic acid (e.g. as described hereinabove).

The present invention also provides a compound of formula (I) or a salt thereof, prepared by (or obtainable by) a process as defined herein.

### Medical Uses

The present invention also provides a compound of formula (I) or a pharmaceutically acceptable salt thereof for use as an active therapeutic substance in a mammal such as a human. The compound or salt can be for use in the treatment and/or prophylaxis of any of the diseases/conditions described herein (e.g. for use in the treatment and/or prophylaxis of an inflammatory and/or allergic disease in a mammal such as a human; or e.g. for use in the treatment and/or prophylaxis of cognitive impairment (e.g. in a neurological disorder such as Alzheimer's disease) or depression in a mammal such as a human) and/or can be for use as a phosphodiesterase 4 (PDE4) inhibitor. “Therapy” may include treatment and/or prophylaxis.

The compound or salt is in particular for use in the treatment and/or prophylaxis of an inflammatory and/or allergic skin disease, such as atopic dermatitis or psoriasis (in particular atopic dermatitis), in a mammal such as a human.

Also provided is the use of a compound of formula (I) or a pharmaceutically acceptable salt thereof in the manufacture of a medicament (e.g. pharmaceutical composition) for the treatment and/or prophylaxis of any of the diseases/conditions described herein in a mammal such as a human, e.g. for the treatment and/or prophylaxis of an inflammatory and/or allergic disease in a mammal such as a human, or e.g. for the treatment and/or prophylaxis of cognitive impairment (e.g. in a neurological disorder such as Alzheimer's disease) or depression in a mammal.

Also provided is a method of treatment and/or prophylaxis of any of the diseases/conditions described herein in a mammal (e.g. human) in need thereof, e.g. a method of treatment and/or prophylaxis of an inflammatory and/or allergic disease, cognitive impairment (e.g. in a neurological disorder such as Alzheimer's disease or schizophrenia) or depression in a mammal (e.g. human) in need thereof, which method comprises administering to the mammal (e.g. human) a therapeutically effective amount of a compound of formula (I) as herein defined or a pharmaceutically acceptable salt thereof.

Phosphodiesterase 4 inhibitors are thought to be, or may be, potentially useful in the treatment and/or prophylaxis of a variety of diseases/conditions, especially inflammatory and/or allergic diseases, in a mammal such as a human, for example: asthma, chronic obstructive pulmonary disease (COPD) (e.g. chronic bronchitis and/or emphysema), atopic dermatitis, urticaria, rhinitis (e.g. allergic rhinitis), allergic conjunctivitis, vernal conjunctivitis, eosinophilic granuloma, psoriasis, rheumatoid arthritis, septic shock, ulcerative colitis, Crohn's disease, reperfusion injury of the myocardium and brain, chronic glomerulonephritis, endotoxic shock, adult respiratory distress syndrome, multiple sclerosis, cognitive impairment (e.g. in a neurological disorder such as Alzheimer's disease or schizophrenia), depression, or pain (e.g. inflammatory pain). Ulcerative colitis and/or Crohn's disease are collectively often referred to as inflammatory bowel disease.

Phosphodiesterase 4 inhibitors, e.g. perhaps the compound or salt of the invention, may also be potentially useful in the treatment and/or prophylaxis of anxiety in a mammal such as a human.

In the treatment and/or prophylaxis, with the compound or salt of the invention, the inflammatory and/or allergic disease can for example be chronic obstructive pulmonary disease (COPD), asthma, rheumatoid arthritis, rhinitis (e.g. allergic rhinitis), psoriasis or atopic dermatitis in a mammal (e.g. human).

In particular, with the compound or salt of the invention, the treatment and/or prophylaxis is of an inflammatory and/or allergic skin disease such as psoriasis or atopic dermatitis (in particular atopic dermatitis) in a mammal (e.g. human).

Suitably, the treatment and/or prophylaxis is of atopic dermatitis in a mammal such as a human or pig, in particular in a human, in particular in a human aged 21 years or less, e.g. 18 years or less. For treatment and/or prophylaxis of atopic dermatitis in a mammal, external topical administration to the mammal of the compound of formula (I) or a pharmaceutically acceptable salt thereof (e.g. topical administration to the skin e.g. to skin affected by the atopic dermatitis) is suitably used, though alternatively oral or parenteral administration can be used. For treatment and/or prophylaxis of atopic dermatitis, inhaled administration is usually not suitable.

“Atopic dermatitis” has been proposed to include two general sub-classes: (1) an “allergic (extrinsic)” type of atopic dermatitis which generally occurs in the context of sensitization to environmental allergens and/or which is generally accompanied by elevated serum IgE levels; and (2) an “non-allergic (intrinsic)” type of atopic dermatitis generally with little or no detectable sensitization and/or generally with normal or low serum IgE levels (N. Novak et al., J. Allergy Clin. Immunol., 2003, 112, 252-262; and T. C. Roos et al., Drugs, 2004, 64(23), 2639-2666, see e.g. pages 2640-2641). The compound of formula (I) or the pharmaceutically acceptable salt thereof can therefore be for the treatment and/or prophylaxis of allergic (extrinsic) atopic dermatitis and/or non-allergic (intrinsic) atopic dermatitis in a mammal (e.g. human or pig, in particular a human).

“External topical” administration means topical administration to an external body part (i.e. excluding, for example, the lung or mouth, but including the lips), in particular excluding the eye.

“External topical” administration is for example topical administration to the skin, for example to the skin of an arm, hand, leg, foot, head (e.g. face), neck and/or torso of a mammal such as a human. External topical administration can for example be to those parts of a mammal's skin affected by or susceptible to atopic dermatitis.

For the use of PDE4 inhibitors in atopic dermatitis, see for example:
J. M. Hanifin et al., “Type 4 phosphodiesterase inhibitors have clinical and in vitro anti-inflammatory effects in atopic dermatitis”, J. Invest. Dermatol., 1996, 107(1), 51-56; which reports reductions of inflammatory parameters in atopic dermatitis patients treated with PDE4 inhibitor CP80,633 (0.5% ointment, twice daily topical application);
C. E. M. Griffiths et al., “Randomized comparison of the type 4 phosphodiesterase inhibitor cipamfylline cream, cream vehicle and hydrocortisone 17-butyrate cream for the treatment of atopic dermatitis”, Br. J. Dermatol., 2002, 147(2), 299-307, which reports that cipamfylline (0.15%) cream is significantly more effective than vehicle, but significantly less effective than hydrocortisone 17-butyrate (0.1%) cream, in the treatment of atopic dermatitis patients;
T. C. Roos et al., “Recent advances in treatment strategies for atopic dermatitis”, Drugs, 2004, 64(23), 2639-2666 (see e.g. page 2657 and refs. 201-209 therein);
A. M. Doherty, Current Opinion Chem. Biol., 1999, 3(4), 466-473 (e.g. see p. 470); and
H. J. Dyke et al., Expert Opinion Invest. Drugs, 2002, 11(1), 1-13 (e.g. see p. 7 and refs. 74, 75 and 76 cited therein);
   and references cited in the above references.

For the use of the PDE4 inhibitors SB 207499 (cilomilast) and AWD 12-281 in mouse models of the allergic type of dermatitis, see W. Bäumer et al., Eur. J. Pharmacol., 2002, 446, 195-200 and W. Baumer et al., J. Pharmacy Pharmacol., 2003, 55, 1107-1114.

PDE4 inhibitors, for example cilomilast and roflumilast, are thought to be effective in the treatment of COPD. For example, see S. L. Wolda, Emerging Drugs, 2000, 5(3), 309-319; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; A. M. Vignola, Respiratory Medicine, 2004, 98, 495-503; D. Spina, Drugs, 2003, 63(23), 2575-2594; and references cited in the aforementioned publications; and G. Krishna et al., Expert Opinion on Investigational Drugs, 2004, 13(3), 255-267 (see especially pp. 259-261 and refs. 102-111 and 201 therein).

The PDE4 inhibitor cilomilast (Ariflo™) at 15 mg orally twice daily appears to improve forced expiratory volume in is (FEV₁) in COPD patients (C. H. Compton et al., The Lancet, 2001, vol. 358, 265-270), and appears to have antiinflammatory effects in COPD patients (E. Gamble et al., Am. J. Respir. Crit. Care Med., 2003, 168, 976-982). On cilomilast, see also R. D. Border et al., Chest, 2003, vol. 124 Suppl. 4, p. 170S (abstract) and J. D. Eddleston et al., Am. J. Respir. Crit. Care Med., 2001, 163, A277 (abstract). The PDE4 inhibitor roflumilast appears to show small improvements in FEV₁ in COPD patients (see B. J. Lipworth, The Lancet, 2005, 365, 167-175, and refs 49-50 therein).

COPD is often characterised by the presence of airflow obstruction due to chronic bronchitis and/or emphysema (e.g., see S. L. Wolda, Emerging Drugs, 2000, 5(3), 309-319).

For treatment and/or prophylaxis of COPD or asthma in a mammal (e.g. human), oral, inhaled or parenteral administration to the mammal of the compound of formula (I) or a pharmaceutically acceptable salt thereof is optionally used, e.g. oral or inhaled administration.

PDE4 inhibitors are thought to be effective in the treatment and/or prophylaxis of asthma (e.g. see M. A. Giembycz, Drugs, February 2000, 59(2), 193-212; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; P. J. Barnes, Nature Reviews—Drug Discovery, October 2004, 831-844; B. J. Lipworth, The Lancet, 2005, 365, 167-175; and references cited in the aforementioned publications).

The PDE4 inhibitor roflumilast, given orally at 500 ug once daily for 9 days, is reported to be effective in improving rhinal airflow during the treatment period (compared to placebo), in humans with histories of allergic rhinitis but asymptomatic at screening, and who were challenged with intranasal allergen provocation (pollen extracts) daily beginning the third day of treatment and each time approx. 2 hours after study drug administration (see B. M. Schmidt et al., J. Allergy & Clinical Immunology, 108(4), 2001, 530-536).

For treatment and/or prophylaxis of rhinitis (e.g. allergic rhinitis), intranasal, oral or parenteral administration of the compound of formula (I) or a pharmaceutically acceptable salt thereof is optionally used.

In one optional embodiment, the compound of formula (I) or the pharmaceutically acceptable salt thereof is for the treatment and/or prophylaxis of rhinitis, such as allergic rhinitis (e.g. seasonal allergic rhinitis or perennial allergic rhinitis) or non-allergic rhinitis (e.g. vasomotor rhinitis), in a mammal such as a human.

PDE4 inhibitors are thought to be, or may be, effective in the treatment of rheumatoid arthritis and multiple sclerosis (e.g. see H. J. Dyke et al., Expert Opinion on Investigational Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and references cited in these publications). For rheumatoid arthritis, oral or parenteral administration is optionally used.

PDE4 inhibitors have been suggested as having analgesic properties and thus being effective in the treatment of pain (A. Kumar et al., Indian J. Exp. Biol., 2000, 38(1), 26-30).

In the invention, the treatment and/or prophylaxis can be of cognitive impairment e.g. cognitive impairment in a neurological disorder (such as Alzheimer's disease or schizophrenia); and/or administration of the compound or salt can optionally be oral. For example, the treatment and/or prophylaxis can comprise cognitive enhancement e.g. in a neurological disorder. For cognition background, see for example: H. T. Zhang et al. in: Psychopharmacology, June 2000, 150(3), 311-316 and Neuropsychopharmacology, 2000, 23(2), 198-204; and T. Egawa et al., Japanese J. Pharmacol., 1997, 75(3), 275-81.

PDE4 inhibitors such as rolipram have been suggested as having antidepressant properties (e.g. J. Zhu et al., CNS Drug Reviews, 2001, 7(4), 387-398; O'Donnell, Expert Opinion on Investigational Drugs, 2000, 9(3), 621-625; H. T. Zhang et al., Neuropsychopharmacology, October 2002, 27(4), 587-595; J. M. O'Donnell and H.-T. Zhang, Trends Pharmacol. Sci., March 2004, 25(3), 158-163; and T. E. Renau, Curr. Opinion Invest Drugs, 2004, 5(1), 34-39).

PDE4 inhibition has been suggested for the treatment of inflammatory bowel disease (e.g. ulcerative colitis and/or Crohn's disease), see K. H. Banner and M. A. Trevethick, Trends Pharmacol. Sci., August 2004, 25(8), 430-436.

### Pharmaceutical Compositions, Routes of Administration, and Dosage Regimens

For use in medicine, the compounds or salts of the present invention are usually administered as a pharmaceutical composition.

The present invention therefore provides in a further aspect a pharmaceutical composition comprising a compound of formula (I) or a pharmaceutically acceptable salt thereof and one or more pharmaceutically acceptable carriers and/or excipients.

The pharmaceutical composition can be for use in the treatment and/or prophylaxis of any of the conditions described herein, for example chronic obstructive pulmonary disease (COPD), asthma, rheumatoid arthritis, allergic rhinitis, psoriasis or atopic dermatitis in a mammal (e.g. human).

The invention also provides a method of preparing a pharmaceutical composition comprising a compound of formula (I), as herein defined, or a pharmaceutically acceptable salt thereof, and one or more pharmaceutically acceptable carriers and/or excipients,
the method comprising mixing the compound or salt with the one or more pharmaceutically acceptable carriers and/or excipients.

The invention also provides a pharmaceutical composition prepared by said method.

The compounds of formula (I) and/or the pharmaceutical composition may be administered, for example, by external topical (e.g. skin topical), oral, parenteral (e.g. intravenous, subcutaneous, or intramuscular), inhaled, or nasal administration. Inhaled administration involves topical administration to the lung e.g. by aerosol or dry powder composition.

Accordingly, the pharmaceutical composition can be suitable for (e.g. adapted for) external topical (e.g. skin topical), oral, parenteral (e.g. intravenous, subcutaneous, or intramuscular), inhaled, or nasal administration, e.g. to a mammal such as a human. The pharmaceutical composition can for example be suitable for external topical (e.g. skin topical) or oral administration, e.g. to a mammal such as a human.

The pharmaceutical composition can optionally be in unit dose form. The unit dose form can for example be:

(a) a tablet or capsule for oral administration e.g. for oral administration to a human;
(b) a rupturable or peel-openable sealed dose container containing a dry powder inhalable pharmaceutical composition (e.g. a plurality of which are usually disposed inside a suitable inhalation device);
(c) a vial, ampoule or filled syringe for parenteral administration e.g. comprising a solution or suspension of the compound or pharmaceutically acceptable salt in a suitable carrier such as an aqueous carrier or e.g. containing a lyophilised parenteral pharmaceutical composition (the vial or ampoule can optionally be manufactured using a blow-fill-seal process).

Alternatively, the composition can be in a form adapted for the administration of varying amounts of composition as desired by the user, such as a spreadable or sprayable external topical composition such as a cream, an ointment, a gel, or a liquid.

### Pharmaceutical Compositions Suitable for External Topical Administration

The pharmaceutical composition of the invention can for example be suitable for (e.g. adapted for) external topical administration (e.g. skin topical administration, i.e. topical administration to the skin), for example to a mammal such as a human. The pharmaceutical composition suitable for external topical administration can suitably be for the treatment and/or prophylaxis of atopic dermatitis in a mammal such as a human, e.g. by external topical administration.

“External topical administration” is defined above under the “medical uses” section. External topical administration can for example be to those parts of the skin affected by or susceptible to the disease or condition e.g. atopic dermatitis, in particular in a mammal (e.g. human) suffering from or susceptible to atopic dermatitis.

An external-topical pharmaceutical composition, e.g. skin topical pharmaceutical composition, can for example be an ointment, a cream (usually an oil-in-water or water-in-oil pharmaceutical composition, usually an emulsion), an aqueous gel, or a microemulsion. The pharmaceutical composition can alternatively be a DMSO-containing solution such as a DMSO/acetone solution or DMSO/water solution (DMSO=dimethyl sulfoxide); a DMSO-containing solution can be used for experimental animal tests, but is not usually desirable for use in humans.

An external-topical pharmaceutical composition, e.g. skin topical pharmaceutical composition, of particular interest is an ointment.

In the external-topical pharmaceutical composition, e.g. an ointment or an oil-in-water or water-in-oil composition, the compound of formula (I) [N-{[1,6-diethyl-4-(tetrahydro-2H-pyran-4-ylamino)-1H-pyrazolo[3,4-b]pyridin-5-yl]methyl}-3-methyl-5-isoxazolecarboxamide] or the pharmaceutically acceptable salt thereof can be present in 0.1% to 10%, such as 0.2% to 10%, or 0.2% to 5%, or 0.5% to 5%, in particular 1% to 10% (e.g. about 2%, about 4% or about 6%), or 1% to 5% (e.g. 1.5% to 5% or 1.5% to 5%, such as about 2% or about 4%), or 0.5% to 3% (e.g. 0.5% or about 2%), or 1% to 3% (e.g. about 2%), by weight of the composition (w/w).

In the external-topical pharmaceutical composition of the invention, the compound of formula (I) or the pharmaceutically acceptable salt thereof can in particular comprise or be the compound (i.e. as the “free base” form).

In one optional embodiment, the compound of formula (I) or the pharmaceutically acceptable salt thereof can optionally be in a particle-size-reduced form, for example obtained or obtainable by micronisation. This can be, for example, for use in a pharmaceutical composition suitable for (e.g. adapted for) external topical (e.g. skin topical) administration. See the Particle size reduction sub-section herein, within the Inhalable pharmaceutical compositions section, for more details.

Aqueous solubility: A preliminary screen, which can aim to estimate roughly the aqueous solubility of a compound or salt of the invention, can include (as an approximate summary): (i) creating a ca. 10 mM solution of the compound in DMSO, (ii) diluting a portion of this DMSO solution by mixing about 19 parts by volume of pH 7.4 aqueous phosphate buffered saline (PBS) buffer with 1 part by volume of the ca. 10 mM DMSO solution, (iii) “filtering” the mixture with the aid of centrifugation, and then (iv) measuring the concentration of the dissolved compound in the “filtrate”. Although some DMSO (about 5% by volume) is usually present in this solubility screen “filtrate”, the results can be a very approximate estimate of aqueous solubility, e.g. at room temperature.

Lipophilicity: The clogP (calculated log of the octanol/water partition coefficient (P)) of a particular compound or salt of the invention can estimate the lipophilicity of the compound or salt.

Solubilising and/or skin-penetration-enhancing agents: An external-topical pharmaceutical composition, e.g. an ointment or an oil-in-water cream or water-in-oil cream, can for example include an agent which acts as a skin-penetration enhancer for and/or a solubiliser of the compound of formula (I) or the salt thereof. The skin-penetration-enhancing- and/or solubilising-agent can for example be propylene glycol, diethylene glycol monoethyl ether (e.g. TRANSCUTOL™) and/or caprylocaproyl macrogolglycerides (e.g. LABRASOL™), in particular propylene glycol. The solubiliser and/or skin-penetration enhancer suitably does not comprise DMSO. The solubiliser and/or skin-penetration enhancer is in particular both a solubiliser and skin-penetration enhancer, and/or can be present in 0.5% to 50%, in particular 5% to 50%, for example 7% to 30%, such as 7% to 25%, e.g. about 10% to about 20% (e.g. about 10% or about 20%), by weight of the composition (w/w).

The skin-penetration enhancer is for delivery of the compound of formula (I) or salt thereof (“active agent” or “drug”) through the skin. Solubilization of the drug also helps. The solubilising and/or skin-penetration-enhancing agents should ideally (a) be safe and/or tolerable, (b) have as low a potential for skin irritancy as possible consistent with being an effective skin penetration enhancer, and (c) be compatible with the active pharmaceutical ingredient. Note that the agent optionally functions both as a solubilising agent and a skin-penetration-enhancing agent.

Surfactants: An external-topical pharmaceutical composition, e.g. an ointment or an oil-in-water cream or water-in-oil cream, can, in one embodiment, include a surfactant (e.g. as an emulsifier), for example for achieving emulsification of compositions having two or more phases. The total surfactant content can for example be 0.3% to 20%, e.g. 0.5% to 15% or 0.5% to 12% or 0.5% to 10% or 1% to 12% or 3% to 10%, by weight of the composition (w/w). The surfactant can for example comprise one or more of the following: a polyoxyl C₁₂₋₂₂alkyl ether (e.g. a polyoxyl C₁₄₋₂₀alkyl ether such as polyoxyl cetyl ether or polyoxyl stearyl ether) (e.g. present at 0.5% to 10% w/w, e.g. 2.5% to 10% w/w such as about 5% to about 8% w/w), glycerol monostearate (e.g. Arlacel 165™) (e.g. present at 0.5% to 10% w/w, e.g. about 2% w/w), sorbitan monostearate (e.g. Span 60 ™) (e.g. present at 0.05% to 10% w/w, e.g. about 1% w/w), cetyl alcohol and/or stearyl alcohol (e.g. wherein the total of any cetyl alcohol and any stearyl alcohol present is 0.1% to 15% w/w, e.g. 1% to 10% w/w such as about 2% to about 5% w/w), and sodium dodecyl sulphate (SDS) (e.g. present at 0.3% to 2% w/w such as about 1% w/w). Polyoxyl stearyl ether (steareth) can e.g. be polyoxyl 2 stearyl ether (steareth 2) or polyoxyl 21 stearyl ether (steareth 21).

DMSO-containing solutions: One possible external-topical pharmaceutical composition is a solution of the compound of formula (I) or the pharmaceutically acceptable salt thereof present at ca. 0.5% to ca. 2.5% w/w in a DMSO-containing solvent such as in DMSO/acetone or in DMSO/water; for example a solution of the compound or salt present at ca. 0.5% to ca. 2.5% w/w in DMSO/acetone (1:1). DMSO-containing solutions, often being capable of high skin penetration, are often good experimental pre-clinical formulations for use in animals e.g. pigs, but their likely skin irritancy generally make them less suitable for use in humans such as patients, e.g. atopic dermatitis patients.

### Ointments (and Oil Phase in Ointments and Creams):

An external-topical pharmaceutical composition can for example be an ointment or an oil-in-water cream or water-in-oil cream. An ointment is of particular interest. The ointment or cream typically contains an oil phase (oily ointment base). The oil phase (oily ointment base) typically comprises an oil and/or a fat, for example of a consistency suitable for skin-spreadability.

The oil phase (oily ointment base) can for example comprise (e.g. be) an oil, wherein the oil comprises (e.g. is) white soft paraffin (white petrolatum) and/or a silicone oil and/or a mineral oil (such as liquid paraffin). (Mineral oil can also be used as a solubiliser and/or emollient).

In particular, the oil phase (oily ointment base) comprises (e.g. is) an oil, wherein the oil comprises (e.g. is) white soft paraffin (white petrolatum) and/or a silicone oil.

Preferably, the external-topical pharmaceutical composition is an ointment, and the oil phase (oily ointment base) comprises (e.g. is) an oil, wherein the oil comprises (e.g. consists essentially of) white soft paraffin (white petrolatum) and a silicone oil.

The white soft paraffin (white petrolatum), e.g. in an ointment or cream, can be of various grades, for example (for Penreco supplier) Penreco Regent White™ grade, Penreco Snow White grade, or Penreco Ultima White™ grade; in particular high melting point white petrolatum (high melting point white soft paraffin) (e.g. of Penreco Ultima White™ grade). The white petrolatum can be present at 25% to 99.9% w/w or 45% to 99.5% w/w or 50% to 99.5% w/w or 45% to 99% w/w or 50% to 99% w/w or 45% to 85% w/w or 45% to 75% w/w (i.e. by weight of the composition).

The silicone oil, e.g. in an ointment or cream, in particular in an ointment, can for example be present at: 5% to 60% w/w such as 5% to 50% w/w, in particular 10% to 50% w/w such as 15% to 40% w/w, suitably 20% to 35% w/w such as about 25% w/w (measured as the total silicone oil content, by weight of the composition).

The silicone oil can be solid or liquid. The silicone oil, e.g. in an ointment or cream, can for example comprise (e.g. be): decamethyl-cyclopentasiloxane (e.g. ST-Cyclomethicone 5-NF™, available from Dow Corning), stearoxytrimethylsilane [Me(CH₂)₁₇O—SiMe₃], polydimethylsiloxane (dimethicone), hexamethyldisiloxane (e.g. ca. 0.65 cSt viscosity at 25° C.), octamethyltrisiloxane (e.g. ca. 1.0 cSt viscosity at 25° C.), decamethyltetrasiloxane, dodecamethylpentasiloxane, or hydroxy-terminated polydimethylsiloxane (e.g. ST-Dimethiconol 40 ™, Dow Corning), or mixtures of any of the foregoing. The silicone oil, e.g. in an ointment or cream, can in particular comprise (e.g. be): decamethyl-cyclopentasiloxane, stearoxytrimethylsilane [Me(CH₂)₁₇O—SiMe₃], or polydimethylsiloxane (dimethicone), or mixtures of any of the foregoing. Preferably, the silicone oil, e.g. in an ointment or cream, can comprise (e.g. be) decamethyl-cyclopentasiloxane.

The decamethyl-cyclopentasiloxane can be ST-Cyclomethicone 5-NF™, available from Dow Corning, and which is described by Dow Corning as being a polydimethyl-cyclosiloxane having a decamethyl-cyclopentasiloxane content of >95% and having a octamethyl-cyclotetrasiloxane content of <1.0%. The decamethyl-cyclopentasiloxane can for example be present at 5% to 60% w/w such as 5% to 50% w/w, in particular 10% to 50% w/w such as 15% to 40% w/w, suitably 20% to 35% w/w such as about 25% w/w (i.e. by weight of the composition).

Stearoxytrimethylsilane [Me(CH₂)₁₇O—SiMe₃] can for example be present as a mixture of stearoxytrimethylsilane and stearyl alcohol for example Silky Wax 10™ which is available from Dow Corning. Stearoxytrimethylsilane (and/or stearoxytrimethylsilane and stearyl alcohol mixture), e.g. in an ointment or cream e.g. ointment, can for example be present at 1% to 30% w/w or 2% to 20% W/w or 5% to 20% w/w such as about 10% w/w.

Polydimethylsiloxane (dimethicone), whose structure is given in the Merck Index 12th edition 1996 as Me₃Si—O—[—Si(CH₃)₂—O—]ₙ—SiMe₃, can for example have a viscosity at 25° C. of from about 20 to about 12500 cSt (centistokes), such as a viscosity at 25° C. of from about 20 to about 350 cSt or from about 20 to about 100 cSt. For example, polydimethylsiloxane (dimethicone) can have a viscosity at 25° C. of: 20 cSt (+10%) (“dimethicone 20”), 100 cSt (±5%), 350 cSt (±5%) (“dimethicone 350”), 1000 cSt (±5%), or 12500 cSt (±5%); grades of polydimethylsiloxane having these five different viscosities are available from Dow Corning as Q7-9120™ Silicone Fluid. Polydimethylsiloxane (dimethicone), e.g. in an ointment, can e.g. be present at 0.1% to 15% w/w such as 0.5% to 10% w/w e.g. 0.5% to 5% w/w.

Microcrystalline wax or beeswax or beeswax substitute can alternatively or additionally be used as an oil/fat in the oil phase.

Alternatively or additionally, one or more fats like straight or branched chain mono- or di-alkyl esters such as isopropyl myristate, isopropyl palmitate, diisopropyl adipate, isocetyl stearate, isostearyl isostearate, decyl oleate, butyl stearate, 2-ethylhexyl palmitate, propylene glycol diester of coconut fatty acids, or a mixed ester of 2-ethyl hexanoic acid with a blend of cetyl or stearyl alcohols (e.g. known as Crodamol CAP) may be used in the oil phase (some of these are also solubilisers and/or surfactants). These may be used singly or in combination depending on the properties required.

The oil phase (oily ointment base) can for example be present at 25% to 99.9% w/w or 25% to 99.5% w/w or 25% to 85% w/w (in particular 45% to 99.5% w/w or 45% to 99% w/w, or 50% to 99.5% w/w or 50% to 99% w/w or 50% to 80% w/w, or 70% to 99.5% w/w or 80% to 99.5% w/w) in an ointment (e.g. as an emulsion, or e.g. as a homogeneous single phase (which does not exclude the compound or salt being at least partly in suspension)).

The oil phase (oily ointment base) can for example be present at 25% to 85% w/w (e.g. 35% to 70% w/w) in an water-in-oil cream (e.g. emulsion), or at 8% to 55% w/w (e.g. 10% to 45% w/w) in an oil-in-water cream (e.g. emulsion).

### Example Ointments:

One particular example of an external-topical pharmaceutical composition is an ointment comprising (e.g. consisting essentially of):
the compound of formula (I) [N-{[1,6-diethyl-4-(tetrahydro-2H-pyran-4-ylamino)-1H-pyrazolo[3,4-b]pyridin-5-yl]methyl}-3-methyl-5-isoxazolecarboxamide] or the pharmaceutically acceptable salt thereof present at 0.5% to 10% w/w (in particular 1% to 10% w/w or 1% to 5% w/w, or 1% to 3% w/w, e.g. about 2% w/w);
white petrolatum present at 25% to 99.9% w/w, in particular 45% to 99.5% w/w or 50% to 99.5% w/w or 45% to 99% w/w or 50% to 99% w/w or 45% to 85% w/w or 45% to 75% w/w (e.g. about 60-85% w/w, e.g. about 73-75% w/w) (i.e. by weight of the composition); and
a silicone oil present at: 5% to 60% w/w such as 5% to 50% w/w, in particular 10% to 50% w/w such as 15% to 40% w/w, suitably 20% to 35% w/w such as about 25% w/w (measured as the total silicone oil content, by weight of the composition).

One preferable example an external-topical pharmaceutical composition is an ointment comprising (e.g. consisting essentially of):
the compound of formula (I) [N-{[1,6-diethyl-4-(tetrahydro-2H-pyran-4-ylamino)-1H-pyrazolo[3,4-b]pyridin-5-yl]methyl}-3-methyl-5-isoxazolecarboxamide] or the pharmaceutically acceptable salt thereof present at 0.5% to 10% w/w (in particular 1% to 10% w/w or 1% to 5% w/w, or 1% to 3% w/w, e.g. about 2% w/w);
white petrolatum present at 25% to 99.9% w/w, in particular 45% to 99.5% w/w or 50% to 99.5% w/w or 45% to 99% w/w or 50% to 99% w/w or 45% to 85% w/w or 45% to 75% w/w (e.g. about 60-85% w/w, e.g. about 73-75% w/w) (i.e. by weight of the composition); and
decamethyl-cyclopentasiloxane (e.g. (e.g. ST-Cyclomethicone 5-NF™) present at 5% to 60% w/w such as 5% to 50% w/w, in particular 10% to 50% w/w such as 15% to 40% w/w, suitably 20% to 35% w/w such as about 25% w/w (i.e. by weight of the composition).

One example of an external-topical pharmaceutical composition is an ointment comprising:
the compound of formula (I) or the pharmaceutically acceptable salt thereof present at 0.2% to 5% w/w (e.g. 0.5% to 5% w/w, or 1% to 3% w/w, e.g. about 2% w/w);
an oil phase (oily ointment base) present at 25% to 99% w/w or 50% to 99% w/w or 25% to 85% w/w or 50% to 80% w/w (for example, the oil phase can comprise white petrolatum present at 25 to 75% w/w or 45 to 75% w/w, and optionally also comprising mineral oil (e.g. as solubiliser and emollient) present at 2.5% to 15% w/w such as 4% to 12% w/w);
one or more surfactants (e.g. polyoxyl stearyl ether) present in total at 0.5% to 10% w/w or 3% to 10% w/w; and
one or more agents acting as a skin-penetration enhancer (in particular acting as both a solubiliser and skin-penetration enhancer and/or in particular a hydrophilic skin-penetration enhancer such as propylene glycol) present in total at 0.5% to 50% w/w, such as 5% to 50% w/w or 7% to 30% w/w (e.g. about 20% w/w, e.g. of propylene glycol); and
optionally one or more antioxidants (e.g. butylated hydroxyanisole), e.g. present in total at 0.001 to 2% w/w such as 0.02 to 2% w/w; and
optionally one or more preservatives, e.g. present in total at 0.01 to 4% w/w such as 0.05 to 1% w/w (e.g. methylparaben present at 0.05 to 2% w/w and/or propylparaben present at 0.01 to 2% w/w).

The above example composition, including the oil “phase” and the penetration enhancer, can optionally be a homogeneous single phase. However, in one embodiment of the above example ointment composition, e.g. when using propylene glycol or another hydrophilic solubiliser and penetration enhancer, the oil phase (oily ointment base) and a hydrophilic phase containing the hydrophilic solubiliser and penetration enhancer (e.g. propylene-glycol-containing phase) have been emulsified to form an ointment emulsion.

Ointment compositions having two phases can optionally be prepared using an emulsification process whereby the hydrophilic phase (e.g. propylene-glycol-containing phase) and oil phase are first prepared in separate vessels. The hydrophilic phase can optionally contain a penetration enhancer such as propylene glycol, and optionally some or all of the compound of formula (I) or salt thereof. The oil phase can optionally contain a surfactant. Temperatures of both phases are maintained at elevated temperatures, such as about 45-90° C. or about 45-80° C. or about 55-90° C. or about 55-80° C. (e.g. about 60-65° C.), or from above 70 to 90° C., the oil phase temperature being sufficiently high (e.g. from above 70 to 90° C.) to melt the oil phase. While hot, one phase is added to another while mixing, e.g. using a high shear mixer, to effect emulsification, optionally keeping the temperature above 70° C. such as from above 70 to 90° C. The resulting ointment emulsion is allowed to cool, e.g. to about 15-35° C. such as to about 17-30° C., in particular while the agitation continues e.g. at lower speeds. The ointment emulsion can then optionally be dispensed from the manufacturing vessel and filled into primary packaging, for example tubes or sachets.

Optionally, an ointment can comprise a polyethylene glycol base, e.g. present at 25 to 98% w/w such as 50 to 95% w/w, instead of or as well as an oily ointment base.

Creams: An external-topical pharmaceutical composition can be a cream, e.g. a water-in-oil cream or an oil-in-water cream.

Water-in-oil creams: These usually have an increased aqueous content compared to ointments. In particular, the water-in-oil cream can be a water-in-oil cream emulsion.

That is, in particular, in the water-in-oil cream, an oil phase and an aqueous phase can have been emulsified to form a water-in-oil cream emulsion.

One example of an external-topical pharmaceutical composition is a water-in-oil cream (e.g. cream emulsion) comprising:
the compound of formula (I) or pharmaceutically acceptable salt thereof present at 0.2% to 10% w/w or 0.2% to 5% w/w (in particular 0.5% to 10% w/w or 1% to 10% w/w, such as 0.5% to 5% w/w or 1% to 5% w/w, or 1% to 3% w/w, e.g. about 2% w/w);
an oil phase (oily ointment base) present at 25% to 85% w/w or 35% to 70% w/w (for example comprising white petrolatum present at 25% to 75% w/w or 30% to 65% w/w (e.g. about 40% w/w), and optionally also comprising mineral oil (e.g. as solubiliser and emollient) present at 2.5% to 15% w/w or 4% to 12% w/w, e.g. about 10% w/w);
water present at 2% to 30% w/w, e.g. 5% to 25% or 10% to 22% w/w (e.g. about 20% w/w);
one or more surfactants (e.g. polyoxyl stearyl ether such as polyoxyl 2 stearyl ether) present in total at 0.5% to 12% w/w, such as 3% to 10% w/w (e.g. about 8% w/w); and
one or more agents acting as a skin-penetration enhancer (in particular acting as both a solubiliser and skin-penetration enhancer and/or in particular a hydrophilic skin-penetration enhancer such as propylene glycol) present in total at 0.5% to 50% w/w, such as 5% to 50% w/w or 7% to 30% w/w (e.g. about 20% w/w, e.g. about 20% w/w of propylene glycol).

The above water-in-oil cream can also optionally comprise:
one or more antioxidants (e.g. butylated hydroxyanisole), e.g. present in total at 0.001 to 2% w/w such as 0.02 to 2% w/w; and/or
one or more preservatives, e.g. present in total at 0.01 to 4% w/w such as 0.05 to 1% w/w (e.g. methylparaben present at 0.05 to 2% w/w and/or propylparaben present at 0.01 to 2% w/w).

Oil-in-water creams: These usually have an increased aqueous content compared to ointments and water-in-oil creams. In particular, the oil-in-water cream can be an oil-in-water cream emulsion. That is, in particular, in the oil-in-water cream, an oil phase and an aqueous phase can have been emulsified to form an oil-in-water cream emulsion.

Oil-in-water creams can for example be high-occlusion creams, wherein, after topical administration to the skin, moisture loss from the skin and/or from the cream is reduced or limited by means of sufficiently high coverage of the skin and/or by providing a sufficient barrier at the site of application.

An oil-in-water cream can in particular contain one or more emollients (hydrating agents), such as silicones (e.g. dimethicone, e.g. dimethicone 360 or dimethicone 20), a high-viscosity wax such as microcrystalline wax, and/or mineral oil.

In an oil-in-water cream, suitably there is a sufficiently high water content, for example wherein the water is present in 15% to 60% w/w, 20% to 50% w/w, or 25% to 40% w/w.

One example of an external-topical pharmaceutical composition is a oil-in-water cream (e.g. cream emulsion) comprising:
the compound of formula (I) or pharmaceutically acceptable salt thereof present at 0.2% to 10% w/w or 0.2% to 5% w/w (in particular 0.5% to 10% w/w or 1% to 10% w/w, such as 0.5% to 5% w/w or 1% to 5% w/w, or 1% to 3% w/w, e.g. about 2% w/w);
an oil phase (oily ointment base) containing one or more ingredients capable of acting as emollients, the oil phase being present at 5% to 60% w/w or in particular 20% to 60% w/w or 30% to 60% w/w such as 30% to 55% w/w;
water present at 12% to 75% w/w or 15% to 75% w/w or 15% to 60% w/w, in particular 15% to 50% w/w or 20% to 40% w/w;
one or more surfactants (e.g. polyoxyl stearyl ether such as polyoxyl 2 stearyl ether) present in total at 0.5% to 12% w/w, e.g. 3% to 10% w/w; and
one or more agents acting as a skin-penetration enhancer (in particular acting as both a solubiliser and skin-penetration enhancer and/or in particular a hydrophilic skin-penetration enhancer such as propylene glycol) present in total at 0.5% to 50% w/w, in particular 5% to 50% w/w or 7% to 25% w/w (e.g. about 20% w/w, e.g. about 20% w/w of propylene glycol).

The above oil-in-water cream can also optionally comprise:
one or more solubilisers (e.g. isopropyl myristate), e.g. present at 0.5% to 20% w/w, e.g. 3 to 12% w/w; and/or
one or more buffers (e.g. citric acid and/or dibasic sodium phosphate), e.g. present in total at 0.05 to 5% w/w.

In the above example oil-in-water cream composition, the oil phase can in particular comprise mineral oil (e.g. as emollient and solubiliser) present at 15% to 50% w/w or 20% to 45% w/w, and/or can in particular comprise a high-viscosity wax such as microcrystalline wax (e.g. as emollient) present at 5% to 25% w/w such as 8% to 15% w/w, and/or can in particular comprise a silicone (such as dimethicone e.g. dimethicone 360 or dimethicone 20, e.g. as emollient) present at 0.5% to 20% such as 0.5% to 10% or 1% to 5% w/w.

In the above example oil-in-water cream composition, the one or more surfactants can for example comprise: glycerol monostearate present at 0.5% to 10% w/w, and/or sorbitan monostearate present at 0.05% to 10% w/w, and/or [cetyl alcohol and/or stearyl alcohol] present in total at 0.1% to 15% or 1 to 10% w/w.

Cream emulsions, e.g. water-in-oil or oil-in-water cream emulsions, can generally be prepared by a process in which an aqueous phase is prepared, e.g. prepared before emulsification. The aqueous phase usually contains water and a solubiliser and/or skin-penetration enhancer such as propylene glycol, and optionally contains some or all of the compound of formula (I) or salt thereof, and/or optionally contains surfactant. The oil phase, e.g. containing white petrolatum and/or mineral oil, and/or optionally containing surfactant, can be prepared in a separate vessel. Temperatures of both phases are suitably maintained at (or heated to) elevated temperatures, such as about 45-90° C. or about 45-80° C. or about 45-75° C., for example about 55-90° C. or about 55-80° C. or about 55-75° C. (in particular at about 60-65° C.), or e.g. from above 70 to 90° C., the oil phase temperature being sufficiently high (e.g. about 45-90° C. or about 55-90° C. or from above 70 to 90° C.) to melt the oil phase. While hot, one phase is suitably added to another while mixing, e.g. using a high shear mixer, to effect emulsification, for example keeping the temperature 45° C. or above, or 55° C. or above such as above 70° C. e.g. from above 70 to 90° C. The resulting emulsion is typically allowed to cool, e.g. to about 15-35° C. such as to about 17-30° C. (e.g. to about 17-22° C.) or to about 18-30° C., for example while the agitation continues e.g. at lower speeds. The cream emulsion can then optionally be dispensed from the manufacturing vessel and filled into primary packaging, for example tubes or sachets.

Typically, a pharmaceutical composition of the invention suitable for external topical administration can be administered once daily, twice daily or more than twice daily, to external body part(s), e.g. on the skin such as at a site of diseased skin, e.g. skin suffering from atopic dermatitis.

### Pharmaceutical Compositions Suitable for Oral or Parenteral Administration

A pharmaceutical composition suitable for oral administration can be liquid or solid; for example it can be a syrup, suspension or emulsion, a tablet, a capsule or a lozenge.

A liquid formulation (e.g. oral) can generally consist of a suspension or solution of the compound or pharmaceutically acceptable salt in a suitable pharmaceutically acceptable liquid carrier(s), for example an aqueous solvent such as water, ethanol or glycerine, or a non-aqueous solvent, such as polyethylene glycol or an oil. The formulation may also contain a suspending agent, preservative, flavouring and/or colouring agent.

In one embodiment, the pharmaceutical composition is in unit dose form, such as a tablet or capsule for oral administration, e.g. for oral administration to a human.

A pharmaceutical composition suitable for oral administration being a tablet can comprise one or more pharmaceutically acceptable carriers and/or excipients suitable for preparing tablet formulations. The carrier can for example be or include lactose, cellulose (for example microcrystalline cellulose), or mannitol. The tablet can also or instead contain one or more pharmaceutically acceptable excipients, for example a binding agent such as hydroxypropylmethylcellulose or povidone (polyvinylpyrrolidone), a lubricant e.g. an alkaline earth metal stearate such as magnesium stearate, and/or a tablet disintegrant such as sodium starch glycollate, croscarmellose sodium, or crospovidone (cross-linked polyvinylpyrrolidone). The pharmaceutical composition being a tablet can be prepared by a method comprising the steps of: (i) mixing the compound of formula (I), as herein defined, or a pharmaceutically acceptable salt thereof, with the one or more pharmaceutically acceptable carriers and/or excipients, (ii) compressing the resulting mixture (which is usually in powder form) into tablets, and (iii) optionally coating the tablet with a tablet film-coating material.

A pharmaceutical composition suitable for oral administration being a capsule can be prepared using encapsulation procedures. For example, pellets or powder containing the active ingredient can be prepared using a suitable pharmaceutically acceptable carrier and then filled into a hard gelatin capsule. Alternatively, a dispersion or suspension can be prepared using any suitable pharmaceutically acceptable carrier, for example an aqueous gum or an oil and the dispersion or suspension then filled into a soft gelatin capsule.

In a pharmaceutical composition for oral administration of the invention, the compound of formula (I) or the pharmaceutically acceptable salt thereof can in one embodiment comprise (e.g. be) the compound of the invention (i.e. as the “free base” form).

A pharmaceutical composition suitable for (e.g. adapted for) parenteral (e.g. intravenous, subcutaneous, or intramuscular) administration can comprise a solution or suspension of the compound or pharmaceutically acceptable salt in a sterile pharmaceutically acceptable aqueous carrier (e.g. sterile water) or parenterally acceptable oil. Alternatively, the solution can be lyophilised. A lyophilised pharmaceutical composition suitable for (e.g. adapted for) parenteral administration may, in use, optionally be reconstituted with a suitable solvent, e.g. sterile water or a sterile parenterally acceptable aqueous solution, just prior to administration.

### Pharmaceutical Compositions Suitable for Inhalable or Intranasal Administration, and Particle-Size Reduction

Compositions suitable for (e.g. adapted for) nasal or inhaled administration may conveniently be formulated as aerosols, drops, gels or dry powders.

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it can contain a suitable propellant under pressure such as compressed air, carbon dioxide, or an organic propellant such as a chlorofluorocarbon (CFC) or hydrofluorocarbon (HFC). Suitable CFC propellants include dichlorodifluoromethane, trichlorofluoromethane and dichlorotetrafluoroethane. Suitable HFC propellants include 1,1,1,2,3,3,3-heptafluoropropane and 1,1,1,2-tetrafluoroethane. The aerosol dosage forms can also take the form of a pump-atomiser.

### Particle Size Reduction of Compound of Formula (I) or Salt Thereof.

In, for example, pharmaceutical compositions suitable (e.g. adapted for) inhaled administration, the compound or salt of formula (I) can be in a particle-size-reduced form. The size-reduced form can for example be obtained or obtainable by micronisation. Micronisation usually involves subjecting the compound/salt to collisional and/or abrasional forces in a fast-flowing circular or spiral/vortex-shaped airstream often including a cyclone component. The particle size of the size-reduced (e.g. micronised) compound or salt can be defined by a D50 value of about 0.5 to about 10 microns, e.g. about 1 to about 7 microns or about 1 to about 5 microns (e.g. as measured using laser diffraction). For example, the compound or salt of formula (I) can have a particle size defined by: a D10 of about 0.3 to about 3 microns (e.g. about 0.5 to about 2 microns, or about 1 micron), and/or a D50 of about 0.5 to about 10 microns or about 1 to about 7 microns or (e.g. about 1 to about 5 microns or about 2 to about 5 microns or about 2 to about 4 microns), and/or a D90 of about 1 to about 30 microns or about 2 to about 20 microns or about 2 to about 15 microns or about 3 to about 15 microns (e.g. about 5 to about 15 microns or about 5 to about 10 microns or about 2 to about 10 microns); for example as measured using laser diffraction.

In particle size measurements, D90, D50 and D10 respectively mean that 90%, 50% and 10% of the material is less than the micron size specified. D50 is the median particle size. DV90, DV50 and DV10 respectively mean that 90%, 50% and 10% by volume of the material is less than the micron size specified. DM90, DM50 and DM10 respectively mean that 90%, 50% and 10% by weight of the material is less than the micron size specified.

Laser diffraction measurement of particle size can use a dry method (wherein a suspension of the compound/salt in an airflow crosses the laser beam) or a wet method [wherein a suspension of the compound/salt in a liquid dispersing medium, such as isooctane or (e.g. if compound is soluble in isooctane) 0.1% Tween 80 in water, crosses the laser beam]. With laser diffraction, particle size can for example be calculated using the Fraunhofer calculation; and/or optionally a Malvern Mastersizer or Sympatec apparatus is used for measurement. For example, particle size measurement and/or analysis by laser diffraction can use any or all of (e.g. all of) the following: a Malvern Mastersizer longbed version, a dispersing medium of 0.1% Tween 80 in water, a stir rate of ca. 1500 rpm, ca. 3 mins sonification prior to final dispersion and analysis, a 300 RF (Reverse Fourier) lens, and/or the Fraunhofer calculation with Malvern software.

An illustrative non-limiting example of a small-scale micronisation process is now given:

### Micronisation Example: Micronisation of a Compound or Salt of One of the Examples

Purpose: To micronise a compound or salt of one of the Examples (described hereinafter), e.g. in an amount of approximately 600-1000 mg thereof, using a Jetpharma MC1 micronizer.
The parent (unmicronised) and micronised materials are analyzed for particle size by laser diffraction and crystallinity by PXRD.

### Micronisation Example: Equipment and Material

| Equipment/material | Description and specification |
|---|---|
| Jetpharma MC1 Micronizer | Nitrogen supply: Air tank with |
| | 275 psi rate tubing |
| Analytical balance | Sartorius Analytical |
| Top loader balance | Mettler PM400 |
| Digital Caliper | VWR Electronic caliper |
| Materials to be micronised | a compound or salt of one of |
| (Procedure 1) | the Examples |
| Materials to be micronised | a compound or salt of one of |
| (Procedure2) | the Examples |

The Jetpharma MC1 Micronizer comprises a horizontal disc-shaped milling housing having: a tubular compound inlet (e.g. angled at ca. 30 degrees to the horizontal) for entry of a suspension of unmicronised compound of formula (I) or salt in a gasflow, a separate gas inlet for entry of gases, a gas outlet for exit of gases, and a collection vessel (micronizer container) for collecting micronised material. The milling housing has two chambers: (a) an outer annular chamber in gaseous connection with the gas inlet, the chamber being for receiving pressurised gas (e.g. air or nitrogen), and (b) a disc-shaped inner milling chamber within and coaxial with the outer chamber for micronising the input compound/salt, the two chambers being separated by an annular wall. The annular wall (ring R) has a plurality of narrow-bored holes connecting the inner and outer chambers and circumferentially-spaced-apart around the annular wall. The holes opening into the inner chamber are directed at an angle (directed part-way between radially and tangentially), and in use act as nozzles directing pressurised gas at high velocity from the outer chamber into the inner chamber and in an inwardly-spiral path (vortex) around the inner chamber (cyclone). The compound inlet is in gaseous communication with the inner chamber via a nozzle directed tangentially to the inner chamber, within and near to the annular wall/ring R. Upper and lower broad-diameter exit vents in the central axis of the inner milling chamber connect to (a) (lower exit) the collection vessel which has no air outlet, and (b) (upper exit) the gas outlet. Inside and coaxial with the tubular compound inlet and longitudinally-movable within it is positioned a venturi inlet (V) for entry of gases. The compound inlet also has a bifurcation connecting to an upwardly-directed material inlet port for inputting material.

In use, the narrow head of the venturi inlet (V) is suitably positioned below and slightly forward of the material inlet port, so that when the venturi delivers pressurised gas (e.g. air or nitrogen) the feed material is sucked from the material inlet port into the gas stream through the compound inlet and is accelerated into the inner milling chamber tangentially at a subsonic speed. Inside the milling chamber the material is further accelerated to a supersonic speed by the hole/nozzle system around the ring (R) (annular wall) of the milling chamber. The nozzles are slightly angled so that the acceleration pattern of the material is in the form of an inwardly-directed vortex or cyclone. The material inside the milling chamber circulates rapidly and particle collisions occur during the process, causing larger particles to fracture into smaller ones. “Centrifugal” acceleration in the vortex causes the larger particles to remain at the periphery of the inner chamber while progressively smaller particles move closer to the centre until they exit the milling chamber, generally through the lower exit, at low pressure and low velocity. The particles that exit the milling chamber are heavier than air and settle downward through the lower exit into the collection vessel (micronizer container), while the exhaust gas rises (together with a minority of small particles of micronised material) and escapes into the atmosphere at low pressure and low velocity.

### Micronisation Example: Procedure:

The micronizer is assembled. The narrow head of the venturi inlet is positioned below and slightly forward of the material inlet port and is measured with a micro-caliper to make sure that it is inserted correctly. The ring (R) and venturi (V) pressures are adjusted according to the values specified in the experimental design (refer to experimental section below) by adjusting the valves on the pressure gauges on the micronizer. The setup is checked for leakage by observing if there is any fluctuation in the reading of the pressure gauges.

Note that the venturi (V) pressure is kept at least 2 bars greater than the ring (R) pressure to prevent regurgitation of material, e.g. outwardly from the material inlet port.

Balance performance is checked with calibration weights. Specified amount of the parent material is fed into the input container of the micronizer using a spatula. The input container plus material is weighed. The equipment pressure is monitored during the micronization process.

Upon completion of the micronising run, the nitrogen supply is shut off and the micronised material is allowed to settle into the micronizer container. The micronised powder in the micronizer container (collection vessel) and the cyclone (above the recovery vessel) are collected together into a pre-weighed and labelled collection vial. The weight of the micronised material is recorded. The input container is re-weighed in order to calculate the amount of input material by difference. The micronizer is disassembled and residual PDE4 compound on the micronizer inner surface is rinsed with 70/30 isopropyl alcohol/water and collected into a flask. The micronizer is then thoroughly cleaned in a Lancer washing machine and dried before subsequent runs are performed.

### Micronisation Example Optional Experimental Parameters

### Procedure 1: Optional Experimental Parameters

This experiment, Procedure 1, can optionally be carried out generally using a procedure and an apparatus generally as described above or similar to those described, using generally the following experimental parameters:

| | | Venturi |
|---|---|---|
| | Material | Pressure (V)/ |
| | input | ring (R) |
| Procedure | amount | Pressure |
| no. | (g) | (bar) |
| 1 | ca. 0.9 g | V = 5 to 7 bar |
| | | R = 3 to 4 bar |

% yield = [(Material from collection vessel + Material from cyclone)/Material input amount] × 100.

The above optional parameters can be varied using the skilled person's knowledge.

### Procedure 2: Optional Experimental Parameters

The optional experimental parameters can for example be as follows:

| | | Venturi | |
|---|---|---|---|
| | Material | Pressure (V)/ | |
| Procedure | input | ring (R) | Intended |
| no. | amount (g) | Pressure (bar) | feed-rate |
| 2 | ca. 0.9 g | V = 8 to 10 bar | 180 to 200 mg/min |
| | | R = 5.5 to 6 bar | |

The above optional parameters can be varied using the skilled person's knowledge.

### Dry Powder Inhalable Compositions

For pharmaceutical compositions suitable (e.g. adapted for) inhaled administration, the pharmaceutical composition may for example be a dry powder inhalable composition. Such a composition can comprise a powder base such as lactose or starch, the compound of formula (I) or salt thereof (suitably in particle-size-reduced form, e.g. in micronised form), and optionally a ternary agent such as L-leucine, mannitol, trehalose, magnesium stearate and/or cellobiose octaacetate (e.g. alpha-D-isomer of cellobiose octaacetate, e.g. available from Aldrich). For cellobiose octaacetate and storage stability, see WO 03/088943.

The dry powder inhalable composition can comprise a dry powder blend of lactose and the compound of formula (I) or salt thereof. The lactose can be lactose hydrate e.g. lactose monohydrate and/or can be inhalation-grade and/or fine-grade lactose. The particle size of the lactose can for example be defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. The particle size of the lactose can for example be defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose can be defined by 90% or more of the lactose particles being less than 100-200 microns in diameter, and/or 50% or more of the lactose particles being less than 40-70 microns in diameter. It can be about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 JD Zwolle, Netherlands).

In the dry powder inhalable composition the compound of formula (I) or salt thereof can for example be present in about 0.1% to about 70% (e.g. about 1% to about 50%, e.g. about 5% to about 40%, e.g. about 20 to about 30%) by weight of the composition.

An illustrative non-limiting example of a dry powder inhalable composition follows:

Dry Powder Formulation Example—Dry powder Lactose Blend Preparation

Using a size-reduced e.g. micronised form of the compound of formula (I) or salt thereof (e.g. as prepared in the Micronisation Example herein), the dry powder blend is, for example, prepared by mixing the required amount of the compound/salt (e.g. 10 mg, 1% w/w) with inhalation-grade lactose containing 10% fines (e.g. 990 mg, 99% w/w) in a Teflon™ (polytetrafluoroethene) pot in a Mikro-dismembrator ball-mill (but without a ball bearing) at % speed (ca. 2000-2500 rpm) for about 4 hours at each blend concentration. The Mikro-dismembrator (available from B. Braun Biotech International,

SchwarzenbergerWeg 73-79, D-34212 Melsungen, Germany; www.bbraunbiotech.com) comprises a base with an upwardly-projecting and sidewardly-vibratable arm to which is attached the Teflon™ pot. The vibration of the arm achieves blending.

Other blends can include: 10% w/w compound/salt (50 mg)+90% w/w lactose (450 mg, inhalation-grade lactose containing 10% fines).

Serial dilution of the 1% w/w blend can achieve e.g. 0.1% and 0.3% w/w blends.

### Dry Powder Inhalation Devices

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container can be rupturable or peel-openable on demand and the dose, e.g. of the dry powder composition, can be administered by inhalation via a device such as the DISKUS™ device, marketed by GlaxoSmithKline. The DISKUS™ inhalation device can e.g. be substantially as described in GB 2,242,134 A. In such device at least one container for the pharmaceutical composition in powder form (the at least one container in particular being a plurality of sealed dose containers mounted longitudinally in a strip or ribbon) is defined between two members peelably secured to one another; the device comprises: means defining an opening station for the said at least one container; means for peeling the members apart at the opening station to open the container; and an outlet, communicating with the opened container, through which a user can inhale the pharmaceutical composition in powder form from the opened container.

### Dosage Regimens

In a pharmaceutical composition suitable for (e.g. adapted for) external topical administration, e.g. an ointment or an oil-in-water or water-in-oil composition, the compound of formula (I) or the pharmaceutically acceptable salt thereof can be present in 0.1% to 10%, such as 0.2% to 10% or 0.2% to 5%, or 0.5% to 10% or 0.5% to 5%, or 1% to 10% or 1% to 5%, or 0.5% to 3%, or 1% to 3% (e.g. about 0.5% or in particular about 2%), by weight of the composition (w/w). Typically, an external-topical pharmaceutical composition can be administered once daily, twice daily or more than twice daily, to external body part(s), e.g. to the skin such as at a site of diseased skin. The amount administered is usually such as substantially to cover the site(s) of diseased skin.

A pharmaceutical composition of the invention can e.g. be in unit dose form such as a tablet or capsule for oral administration, e.g. for oral administration to a human.

In the pharmaceutical composition of the invention, a or each dosage unit for oral or parenteral administration can for example contain from 0.01 to 3000 mg, such as 0.5 to 1000 mg, of a compound of the formula (I) or a pharmaceutically acceptable salt thereof, calculated as the free base. A or each dosage unit for nasal or inhaled administration can for example contain from 0.001 to 50 mg, such as 0.01 to 5 mg, of a compound of the formula (I) or a pharmaceutically acceptable salt thereof, calculated as the free base.

When a parenteral or oral composition is used, a pharmaceutically acceptable compound or salt of the invention can for example be administered to a mammal (e.g. human) in a daily oral or parenteral dose of 0.001 mg to 50 mg per kg body weight per day (mg/kg/day), for example 0.01 to 20 mg/kg/day or 0.03 to 10 mg/kg/day or 0.1 to 2 mg/kg/day, of the compound of the formula (I) or a pharmaceutically acceptable salt thereof, calculated as the free base.

When a parenteral or oral composition is used, a pharmaceutically acceptable compound or salt of the invention can for example be administered to a mammal (e.g. human) in a daily nasal or inhaled dose of: 0.0001 to 5 mg/kg/day or 0.0001 to 1 mg/kg/day, e.g. 0.001 to 1 mg/kg/day or 0.001 to 0.3 mg/kg/day or 0.001 to 0.1 mg/kg/day or 0.005 to 0.3 mg/kg/day, of the compound of the formula (I) or a pharmaceutically acceptable salt thereof, calculated as the free base.

The pharmaceutically acceptable compounds or salts of the invention can for example be administered to a human in a daily dose (for an adult patient) of, for example, an oral or parenteral dose of 0.01 mg to 3000 mg per day or 0.5 to 1000 mg per day e.g. 2 to 500 mg per day, or a nasal or inhaled dose of 0.001 to 50 mg per day or 0.01 to 30 mg per day or 0.01 to 5 mg per day or 0.02 to 2 mg per day, of the compound of the formula (I) or a pharmaceutically acceptable salt thereof, calculated as the free base.

### Combinations

The compounds, salts and/or pharmaceutical compositions according to the invention may also be used in combination with another therapeutically active agent, for example, a 2 adrenoreceptor agonist, an anticholinergic compound (e.g. muscarinic (M) receptor antagonist), an anti-histamine, an anti-allergic, an anti-inflammatory agent, an antiinfective agent or an immunosuppressant.

The invention thus provides, in a further aspect, a combination comprising a compound of formula (I) or a pharmaceutically acceptable salt thereof together with another therapeutically active agent, for example, a β₂-adrenoreceptor agonist, an anti-histamine, an anti-allergic, an anti-inflammatory agent, an antiinfective agent or an immunosuppressant.

In one particular embodiment, the β₂-adrenoreceptor agonist is salmeterol (e.g. as racemate or a single enantiomer such as the R-enantiomer), salbutamol, formoterol, salmefamol, fenoterol or terbutaline, or a salt thereof (e.g. pharmaceutically acceptable salt thereof), for example the xinafoate salt of salmeterol, the sulphate salt or free base of salbutamol or the fumarate salt of formoterol. Long-acting β₂-adrenoreceptor agonists are optionally used, especially those having a therapeutic effect over a 12-24 hour period such as salmeterol or formoterol. In particular, the β₂-adrenoreceptor agonist is for inhaled administration, e.g. once per day and/or for simultaneous inhaled administration; and in particular the β₂-adrenoreceptor agonist can be in particle-size-reduced form e.g. as defined herein. The β₂-adrenoreceptor agonist combination can for example be for treatment and/or prophylaxis of COPD or asthma. Salmeterol or a pharmaceutically acceptable salt thereof, e.g. salmeterol xinofoate, is suitably administered to humans at an inhaled dose of 25 to 50 micrograms twice per day (measured as the free base).

Examples of long acting β₂-adrenoreceptor agonists which could be used include those described in WO 02/066422A, WO 03/024439, WO 02/070490 and WO 02/076933.

Particular examples of 2-adrenoreceptor agonists disclosed in WO 02/066422 include:
3-(4-{[6-({(2R)-2-hydroxy-2-[4-hydroxy-3-(hydroxymethyl)-phenyl]ethyl}amino)hexyl]oxy}butyl)benzenesulfonamide and
3-(3-{[7-({(2R)-2-hydroxy-2-[4-hydroxy-3-hydroxymethyl)phenyl]ethyl}-amino)heptyl]oxy}propyl)benzenesulfonamide.

A particular example of a β₂-adrenoreceptor agonist disclosed in WO 03/024439 is:
4-{(1R)-2-[(6-{2-[(2,6-dichlorobenzyl)oxy]ethoxy}hexyl)amino]-1-hydroxyethyl}-2-(hydroxymethyl)phenol.

An anti-histamine usable in a combination of a compound of formula (I) or salt can for example be for oral administration (e.g. as a combined composition such as a combined tablet), and can be for treatment and/or prophylaxis of allergic rhinitis. Examples of anti-histamines include methapyrilene, or H₁ antagonists such as cetirizine, loratadine (e.g. Clarityn™), desloratadine (e.g. Clarinex™) or fexofenadine (e.g. Allegra™).

The invention also provides, in a further aspect, a combination comprising a compound of formula (I) or a pharmaceutically acceptable salt thereof together with an anticholinergic compound, e.g. a muscarinic (M) receptor antagonist such as an M₁, M₂, M₁/M₂, or M₃ receptor antagonist, in particular a M₃ receptor antagonist, such as a M₃ receptor antagonist which selectively antagonises (e.g. antagonises 10 times or more strongly) the M₃ receptor over the M₁ and/or M₂ receptor. For combinations of anticholinergic compounds/muscarinic (M) receptor antagonist with PDE4 inhibitors, see for example WO 03/011274 A2 and WO 02/069945 A2/US 2002/0193393 A1 and US 2002/052312 A1, and some or all of these publications give examples of anticholinergic compounds/muscarinic (M) receptor antagonists which may be used with the compounds of formula (I) or salts, and/or suitable pharmaceutical compositions. For example, the muscarinic receptor antagonist can comprise or be an ipratropium salt (e.g. ipratropium bromide), an oxitropium salt (e.g. oxitropium bromide), or a tiotropium salt (e.g. tiotropium bromide); see e.g. EP 418 716 A1 for tiotropium.

The anticholinergic compound or muscarinic (M) receptor antagonist, e.g. M₃ receptor antagonist, can for example be for inhaled administration, in particular in particle-size-reduced form e.g. as defined herein. In one optional embodiment, both the muscarinic (M) receptor antagonist and the compound of formula (I) or the pharmaceutically acceptable salt thereof are for inhaled administration. For example, the anticholinergic compound or muscarinic receptor antagonist and the compound of formula (I) or salt are optionally for simultaneous administration. The muscarinic receptor antagonist combination is optionally for treatment and/or prophylaxis of COPD.

Other possible combinations include, for example, a combination comprising a compound of formula (I) or a pharmaceutically acceptable salt thereof together with another anti-inflammatory agent such as an anti-inflammatory corticosteroid; or a non-steroidal anti-inflammatory drug (NSAID) such as a leukotriene antagonist (e.g. montelukast), an iNOS inhibitor, a tryptase inhibitor, a elastase inhibitor, a beta-2 integrin antagonist, a adenosine 2a agonist, a CCR3 antagonist, or a 5-lipoxogenase inhibitor; or an antiinfective agent (e.g. an antibiotic or an antiviral). An iNOS inhibitor can e.g. be for oral administration. Examples of iNOS inhibitors (inducible nitric oxide synthase inhibitors) include those disclosed in WO 93/13055, WO 98/30537, WO 02/50021, WO 95/34534 and WO 99/62875.

Example combinations, in particular for external topical administration (e.g. versus atopic dermatitis), include, for example, a combination comprising a compound of formula (I) or a pharmaceutically acceptable salt thereof together with an immunosuppressant, e.g. a calcineurin inhibitor such as pimecrolimus or tacrolimus. The immunosuppressant can in particular be an externally-topically administrable immunosuppressant such as pimecrolimus (e.g. pimecrolimus at ca. 1% w/w concentration in a topical composition such as a cream, and/or e.g. Elidel™) or tacrolimus (e.g. tacrolimus at from about 0.03% to about 0.1% w/w concentration in a topical composition such as an ointment, and/or e.g. Protopic™). The externally-topically administrable immunosuppressant can be administered or administrable in a external-topical composition separately from the compound or salt of the invention, or it can be contained with the compound of formula (I) or pharmaceutically acceptable salt in a combined externally-topically-administrable composition.

For external topical administration, e.g. versus an inflammatory and/or allergic skin disease such as atopic dermatitis or psoriasis, in a combination of the compound or salt of the invention together with an anti-infective agent, the anti-infective agent can include (e.g. be) an externally-topically-administrable antibacterial, such as mupiricin or a salt thereof (e.g. mupiricin calcium salt) (e.g. Bactroban™) or such as an externally-topically-administrable pleuromutilin antibacterial (e.g. retapamulin or a salt thereof, which can be present in about 1% w/w by weight of an externally-topically-administrable pharmaceutical composition, such as an ointment). Alternatively or additionally, for external topical administration, the anti-infective agent can include an externally-topically-administrable antifungal such as clortrimazole, clotrimazole or ketoconazole.

For external topical administration, e.g. versus atopic dermatitis, a combination with an anti-itch compound may optionally be used.

In a combination comprising a compound of formula (I) or a pharmaceutically acceptable salt thereof together with an anti-inflammatory corticosteroid (which can for example be for treatment and/or prophylaxis of asthma, COPD or allergic rhinitis), then optionally the anti-inflammatory corticosteroid is fluticasone propionate (e.g. see U.S. Pat. No. 4,335,121), beclomethasone 17-propionate ester, beclomethasone 17,21-dipropionate ester, dexamethasone or an ester thereof, mometasone or an ester thereof (e.g. mometasone furoate), ciclesonide, budesonide, flunisolide, or a compound as described in WO 02/12266 A1 (e.g. as claimed in any of claims 1 to 22 therein), or a pharmaceutically acceptable salt of any of the above. If the anti-inflammatory corticosteroid is a compound as described in WO 02/12266 A1, then it can for example be Example 1 therein {which is 6α,9α-difluoro-17α-[(2-furanylcarbonyl)oxy]-11β-hydroxy-16α-methyl-3-oxo-androsta-1,4-diene-17β-carbothioic acid S-fluoromethyl ester} or Example 41 therein {which is 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester}, or a pharmaceutically acceptable salt thereof. The anti-inflammatory corticosteroid can for example be for external topical, intranasal or inhaled administration. Fluticasone propionate can be used for inhaled administration to a human, for example either (a) at a dose of 250 micrograms once per day or (b) at a dose of 50 to 250 micrograms twice per day.

Also provided is a combination comprising a compound of formula (I) or a pharmaceutically acceptable salt thereof together with β₂-adrenoreceptor agonist and an anti-inflammatory corticosteroid, for example as described in WO 03/030939 A1. This combination can for example be for treatment and/or prophylaxis of asthma, COPD or allergic rhinitis. The 2-adrenoreceptor agonist and/or the anti-inflammatory corticosteroid can be as described above and/or as described in WO 03/030939 A1. In this “triple” combination, the β₂-adrenoreceptor agonist can e.g. be salmeterol or a pharmaceutically acceptable salt thereof (e.g. salmeterol xinafoate) and the anti-inflammatory corticosteroid can e.g. be fluticasone propionate.

The combinations referred to above may be presented for use in the form of a pharmaceutical composition and thus a pharmaceutical composition comprising a combination as defined above together with one or more pharmaceutically acceptable carriers and/or excipients represent a further aspect of the invention.

The individual compounds of such combinations may be administered either sequentially or simultaneously in separate or combined pharmaceutical composition.

In one embodiment, the combination as defined herein can be for simultaneous inhaled administration and is disposed in a combination inhalation device. Such a combination inhalation device is another aspect of the invention. Such a combination inhalation device can comprise a combined pharmaceutical composition for simultaneous inhaled administration (e.g. dry powder composition), the composition comprising all the individual compounds of the combination, and the composition being incorporated into a plurality of sealed dose containers mounted longitudinally in a strip or ribbon inside the inhalation device, the containers being rupturable or peel-openable on demand; for example such inhalation device can be substantially as described in GB 2,242,134 A (DISKUS™) and/or as described above. Alternatively, the combination inhalation device can be such that the individual compounds of the combination are administrable simultaneously but are stored separately (or wholly or partly stored separately for triple combinations), e.g. in separate pharmaceutical compositions, for example as described in PCT/EP03/00598 filed on 22 Jan. 2003, published as WO 03/061743 (e.g. as described in the claims thereof e.g. claim 1).

The invention also provides a method of preparing a combination as defined herein,
the method comprising either
(a) preparing a separate pharmaceutical composition for administration of the individual compounds of the combination either sequentially or simultaneously, or
(b) preparing a combined pharmaceutical composition for administration of the individual compounds of the combination simultaneously,
wherein the pharmaceutical composition comprises the combination together with one or more pharmaceutically acceptable carriers and/or excipients.

The invention also provides a combination as defined herein, prepared by a method as defined herein.

### Biological Test Methods

### PDE 3, PDE 4B, PDE 4D, PDE 5, PDE 6 In Vitro Assay Methods

The biological activity of the compounds or salts of the invention can be measured in the assay methods shown below, or in generally similar or generally analogous assay methods.

### Possible PDE Enzyme Sources and Literature References

Human recombinant PDE4B, in particular the 2B splice variant thereof (HSPDE4B2B), is disclosed in WO 94/20079 and also M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phoshodiesterase (PDE IV_{D})”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 can be purified from bovine aorta as described by H. Coste and P. Grondin, “Characterisation of a novel potent and specific inhibitor of type V phosphodiesterase”, Biochem. Pharmacol., 1995, 50, 1577-1585.

PDE6 can be purified from bovine retina as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

### Inhibition of PDE 3, PDE 4B, PDE 4D, PDE 5 or PDE 6 Activity: Radioactive Scintillation Proximity Assay (SPA)

The ability of compounds to inhibit catalytic activity at PDE4B or 4D (human recombinant), PDE3 (from bovine aorta), PDE5 (human recombinant) or PDE6 (from bovine retina) can optionally be determined by Scintillation Proximity Assay (SPA) in a 96-well format.

Test compounds (as a solution in DMSO, suitably about 2 microlitre (ul) volume of DMSO solution) are preincubated at ambient temperature (room temperature, e.g. 19-23° C.) in Wallac Isoplates (code 1450-514) with PDE enzyme in 50 mM Tris-HCl buffer pH 7.5, 8.3 mM MgCl₂, 1.7 mM EGTA, 0.05% (w/v) bovine serum albumin for 10-30 minutes (usually 30 minutes). The enzyme concentration is adjusted so that no more than 20% hydrolysis of the substrate defined below occurs in control wells without compound, during the incubation. For the PDE3, PDE4B and PDE4D assays, [5′,8-³H]Adenosine 3′,5′-cyclic phosphate (Amersham Pharmacia Biotech, code TRK.559; or Amersham Biosciences UK Ltd, Pollards Wood, Chalfont St Giles, Buckinghamshire HP8 4SP, UK) is added to give 0.05 uCi per well and about 10 nM final concentration. For the PDE5 and PDE6 assays, [8-³H]Guanosine 3′,5′-cyclic phosphate (Amersham Pharmacia Biotech, code TRK.392) is added to give 0.05 uCi per well and about 36 nM final concentration. Plates containing assay mixture, suitably approx. 100 ul volume of assay mixture, are mixed on an orbital shaker for 5 minutes and incubated at ambient temperature for 1 hour. Phosphodiesterase SPA beads (Amersham Pharmacia Biotech, code RPNQ 0150) are added (about 1 mg per well) to terminate the assay. Plates are sealed and shaken and allowed to stand at ambient temperature for 35 minutes to 1 hour (suitably 35 minutes) to allow the beads to settle. Bound radioactive product is measured using a WALLAC TRILUX 1450 Microbeta scintillation counter. For inhibition curves, 10 concentrations (e.g. 1.5 nM-30 uM) of each compound are assayed. Curves are analysed using ActivityBase and XLfit (ID Business Solutions Limited, 2 Ocean Court, Surrey Research Park, Guildford, Surrey GU2 7QB, United Kingdom). Results are expressed as pIC₅₀ values.

In an alternative to the above radioactive SPA assay, PDE4B or PDE4D inhibition can be measured in the following Fluorescence Polarisation (FP) assay:

### Inhibition of PDE4B or PDE4D Activity: Fluorescence Polarisation (FP) Assay

The ability of compounds to inhibit catalytic activity at PDE4B (human recombinant) or PDE4D (human recombinant) can optionally be determined by IMAP Fluorescence Polarisation (FP) assay (IMAP Explorer kit, available from Molecular Devices Corporation, Sunnydale, Calif., USA; Molecular Devices code: R8062) in 384-well format.

The IMAP FP assay is able to measure PDE activity in an homogenous, non-radioactive assay format. The FP assay uses the ability of immobilised trivalent metal cations, coated onto nanoparticles (tiny beads), to bind the phosphate group of Fl-AMP that is produced on the hydrolysis of fluorescein-labelled (Fl) cyclic adenosine mono-phosphate (Fl-cAMP) to the non-cyclic Fl-AMP form. Fl-cAMP substantially does not bind. Binding of Fl-AMP product to the beads (coated with the immobilised trivalent cations) slows the rotation of the bound Fl-AMP and leads to an increase in the fluorescence polarisation ratio of parallel to perpendicular light. Inhibition of the PDE reduces/inhibits this signal increase.

Test compounds (small volume, e.g. ca. 0.5 to 1 microlitres (ul), suitably ca. 0.5 ul, of solution in DMSO) are preincubated at ambient temperature (room temperature, e.g. 19-23° C.) in black 384-well microtitre plates (supplier: NUNC, code 262260) with PDE enzyme in 10 mM Tris-HCl buffer pH 7.2, 1 mM MgCl₂, 0.1% (w/v) bovine serum albumin, and 0.05% NaN₃ for 10-30 minutes. The enzyme level is set by experimentation so that reaction is linear throughout the incubation. Fluorescein adenosine 3′,5′-cyclic phosphate (from Molecular Devices Corporation, Molecular Devices code: R7091) is added to give about 40 nM final concentration (final assay volume usually ca. 20-40 ul, suitably ca. 20 ul). Plates are mixed on an orbital shaker for 10 seconds and incubated at ambient temperature for 40 minutes. IMAP binding reagent (as described above, from Molecular Devices Corporation, Molecular Devices code: R7207) is added (60 ul of a 1 in 400 dilution in binding buffer of the kit stock solution) to terminate the assay. Plates are allowed to stand at ambient temperature for 1 hour. The Fluorescence Polarisation (FP) ratio of parallel to perpendicular light is measured using an Analyst™ plate reader (from Molecular Devices Corporation). For inhibition curves, 10 concentrations (e.g. 1.5 nM-30 uM) of each compound are assayed. Curves are analysed using ActivityBase and XLfit (ID Business Solutions Limited, 2 Ocean Court, Surrey Research Park, Guildford, Surrey GU2 7QB, United Kingdom). Results are expressed as pIC₅₀ values.

In the FP assay, reagents can be dispensed using Multidrop™ (available from Thermo Labsystems Oy, Ratastie 2, PO Box 100, Vantaa 01620, Finland).

For a given PDE4 inhibitor, the PDE4B (or PDE4D) inhibition values measured using the SPA and FP assays can differ slightly.

Biological Data obtained for the compound of the invention (e.g. as prepared in Example 1) (PDE4B and PDE4D inhibitory activity, as an average of 2 or more readings (n=2 or more, e.g. 3) are generally as follows, based on measurements only, generally using FP assay(s) generally as described above or generally similar or generally analogous to those described above. In each of the SPA and FP assays, absolute accuracy of measurement is not possible, and the readings given are generally thought to be accurate only up to very approximately ±0.5 of a log unit, depending on the number of readings made and averaged:

| | Mean pIC₅₀ value |
|---|---|
| | (number (n) of |
| Example 1 data type | readings averaged) |
| PDE4B pIC₅₀ (± about 0.5) (FP assay) | about 9.8 (n = 3) |
| PDE4D pIC₅₀ (± about 0.5) (FP assay) | about 10.0 (n = 3) |

The compound of the invention (e.g. as prepared in Example 1) has also been tested for PDE3, PDE5 and PDE6 inhibition, e.g. using (independently for each PDE type) either a Fluorescence Polarisation (FP) general type of assay or using an SPA general type of assay. The compound of the invention (e.g. as prepared in Example 1) exhibits a larger PDE4B pIC₅₀ value than its PDE3, PDE5 and PDE6 pIC₅₀ values, i.e. it inhibits PDE4B more strongly than it inhibits PDE3, PDE5 and PDE6 (in the particular assays used).

Emesis: Some known PDE4 inhibitors can cause emesis and/or nausea to greater or lesser extents, especially after systemic exposure e.g. after oral administration (e.g. see Z. Huang et al., Current Opinion in Chemical Biology, 2001, 5: 432-438, see especially pages 433-434 and refs cited therein). Therefore, it would be preferable, but not essential, if a PDE4 inhibitory compound or salt of the invention were to cause only limited or manageable emetic side-effects, e.g. after oral or parenteral or external-topical administration. Emetic side-effects can for example be measured by the emetogenic potential of the compound or salt when administered to ferrets; for example one can measure the time to onset, extent, frequency and/or duration of vomiting, retching and/or writhing in ferrets after oral or parenteral administration of the compound or salt. See for example In vivo Assay 3 hereinafter for one optional measurement method for anti-inflammatory effect, emetic side-effects and therapeutic index (TI) in the ferret. See also for example A. Robichaud et al., “Emesis induced by inhibitors of [PDE IV] in the ferret”, Neuropharmacology, 1999, 38, 289-297, erratum Neuropharmacology, 2001, 40, 465-465. However, optionally, emetic side-effects and therapeutic index (TI) after oral administration in rats can be conveniently measured by monitoring the pica feeding behaviour of rats after administration of the compound or salt of the invention (see In Vivo Assay 2 below).

### Other Optional In Vitro Assays:

### Inhibition of TNF-α (TNF-alpha) Production in Human PBMC (Peripheral Blood Mononuclear Cell) Assay (MSD Technology)

This is an optional supplementary test.

A 96-well plate (96 MicroWell™ Plates Nunclon™Δ-High Flange Design, Fisher Scientific UK, Bishop Meadow Road, Loughborough LE 11 5 RG, Leicestershire, UK) is prepared by initially adding to column 1 ca. 10 mM of test compound dissolved in DMSO. For a more potent compound, a more diluted solution in DMSO may be used. The compound is further diluted with DMSO into columns 2 to 9 by 8 successive 3-fold dilutions using the Biomek® FX Laboratory Automation Workstation (Beckman Coulter, Inc., 4300 N. Harbor Boulevard, P.O. Box 3100, Fullerton, Calif. 92834-3100 USA). Column 10 is used as a DMSO negative control (High Signal, 0% response), whilst column 11, which contains 10 mM of the PDE4 inhibitor roflumilast, is used as a positive control (Low Signal, 100% response). About 1 μl (about 1 ul) of compound is transferred to the compound plate using the Biomek® FX.

PBMC cells (peripheral blood mononuclear cells) are prepared from heparinised human blood (using 1% v/v Heparin Sodium 1000 IU/ml Endotoxin Free, Leo Laboratories Ltd., Cashel Road, Dublin 12. Ireland, Cat No: PL0043/0149) from normal volunteers using the Accuspin™ System-Histopaque®-1077 essentially (Sigma-Aldrich Company Ltd., The Old Brickyard New Rd, Gillingham Dorset SP8 4XT). About 20 ml of blood is overlaid onto 15 ml Histopaque® in Accuspin™ tubes. The tube is then centrifuged at about 800 g for ca. 20 minutes. The cells are collected from the interface, washed by centrifugation (ca. 1300 g, ca. 10 minutes) and resuspended in RPM11640 medium (Low endotoxin RPM11640 medium, Cat No: 31870-025, Invitrogen Corporation Invitrogen Ltd, 3 Fountain Drive, Inchinnan Business Park, Paisley PA4 9RF, UK) containing 10% foetal calf serum, 1% L-glutamine (Invitrogen Corporation, Cat No: 25030024) and 1% penicillin/streptomycin (Invitrogen Corporation, Cat No: 15140-122). Viable cells are counted by trypan blue staining and diluted to 1×10⁶ viable cells/ml. About 50 μl (about 50 ul) of diluted cells and about 75 μl (about 75 ul) of LPS (ca. 1 ng/ml final; Sigma Cat No: L-6386) are added to the compound plate, which is then incubated at 37° C., 5% CO₂, for 20 hours.

The supernatant is removed and the concentrations of TNF-α are determined by electrochemiluminescence assay using the Meso Scale Discovery (MSD) technology (Meso Scale Discovery, 9238 Gaither Road, Gaithersburg, Md. 20877, USA). See the “TNF-α (TNF-alpha) MSD Assay” described below for typical details.

Results can be expressed as pIC50 values for inhibition of TNF-α (TNF-alpha) production in PBMCs, and it should be appreciated that these results can be subject to variability or error.

### Inhibition of TNFα (TNF-alpha) Production in Human PBMC (Peripheral Blood Mononuclear Cell) Assay (IGEN Technology)

This is an optional supplementary test.

Test compounds are prepared as a ca. 10 mM stock solution in DMSO and a dilution series prepared in DMSO with 8 successive 3-fold dilutions, either directly from the 10 mM stock solution or from a more dilute solution in DMSO. The compound is added to assay plates using a Biomek Fx liquid handling robot.

PBMC cells (peripheral blood mononuclear cells) are prepared from heparinised human blood from normal volunteers by centrifugation on histopaque at ca. 1000 g for ca. 30 minutes. The cells are collected from the interface, washed by centrifugation (ca. 1300 g, ca. 10 minutes) and resuspended in assay buffer (RPMI 1640 containing 10% foetal calf serum, 1% L-glutamine and 1% penicillin/streptomycin) at 1×10⁶ cells/ml. Ca. 50 μl (ca. 50 ul) of cells are added to microtitre wells containing ca. 0.5 or ca. 1.0 μl (ul) of an appropriately diluted compound solution. Ca. 75 μl (ul) of LPS (lipopolysaccharide) (ca. 1 ng/ml final) is added and the samples are incubated at 37° C., 5% CO₂, for 20 hours. The supernatant is removed and the concentrations of TNF-α are determined by electrochemiluminescence assay using the IGEN technology or by ELISA (see below).

Results can be expressed as pIC50 values for inhibition of TNF-α (TNF-alpha) production in PBMCs, and it should be appreciated that these results can be subject to variability or error.

### Inhibition of TNFα (TNF-alpha) Production in Human Whole Blood

This is an optional supplementary test, e.g. for potentially orally-administrable PDE4 inhibitors. Also, as the assay may measure the effect of PDE4 inhibitors after loss by protein binding, it might possibly also be relevant to externally-topically-administrable PDE4 inhibitors as protein-binding-loss of compound is possible during transport through the skin.

Test compounds are prepared as a ca. 10 mM stock solution in DMSO and a dilution series prepared in DMSO with 8 successive 3-fold dilutions, either directly from the 10 mM stock solution or from a more dilute solution in DMSO. The compound is added to assay plates using a Biomek Fx liquid handling robot.

Heparinised blood drawn from normal volunteers is dispensed (ca. 100 μl=ca. 100 ul) into microtitre plate wells containing ca. 0.5 or ca. 1.0 μl (ul, microlitres) of an appropriately diluted test compound solution. After ca. 1 hr incubation at ca. 37° C., 5% CO₂, ca. 25 μl (ca. 25 ul) of LPS (lipopolysaccharide) solution (S. typhosa) in RPMI 1640 (containing 1% L-glutamine and 1% Penicillin/streptomycin) is added (ca. 50 ng/ml final). The samples are incubated at ca. 37° C., 5% CO₂, for ca. 20 hours, and ca. 100 μl (ca. 100 ul) physiological saline (0.138% NaCl) is added, and diluted plasma is collected using a Platemate or Biomek FX liquid handling robot after centrifugation at ca. 1300 g for ca. 10 min. Plasma TNFα content is determined by electrochemiluminescence assay using the MSD technology (see below), the IGEN technology (see below) or by enzyme linked immunosorbant assay (ELISA) (see below).

Results can be expressed as pIC50 values for inhibition of TNF-α (TNF-alpha) production in Human Whole Blood, and it should be appreciated that these results can be subject to variability or error.

### Human Whole Blood Assay Results:

For the compound of the invention (e.g. as prepared in Example 1), and generally when using the one of the above assays, or a generally similar or generally analogous assay, the mean and/or measured pIC50 values for inhibition of TNF-α (TNF-alpha) production in Human Whole Blood are generally as follows (subject to variability or error):

| | Whole Blood, Mean pIC₅₀ | |
|---|---|---|
| Example | (MSD assay) | Whole Blood, Mean pIC₅₀ |
| number | (n = no. of measurements) | (IGEN assay) |
| 1 | about 9.4 (n = about 3 or 4) | about 8.7 (n = about 4) |
| | (individual pIC₅₀ test values are: | (individual pIC₅₀ test |
| | about 9.19, about 9.50, & about | values range from about |
| | 9.50; and also about >9.82) | 8.58 to about 8.90) |

### TNF-α (TNF-alpha) MSD Assay

Using the Biomek FX, 25 μl (25 ul) of MSD Human Serum Cytokine Assay Diluent (Meso Scale Discovery, 9238 Gaither Road, Gaithersburg, Md. 20877) is added to a 96-well High-Bind MSD plate pre-coated with anti-hTNF alpha capture antibody (MA6000) and then incubated for 24 hours at 4° C. to prevent non-specific binding. About 20 μl (ul) of supernatant from the PBMC plate or about 40 μl (ul) of supernatant from the whole blood (WB) plate are then transferred from columns 1-11 to columns 1-11 of the MSD plate using the Biomek FX. About 20 μl (ul) of TNF-α standard (Cat No. 210-TA; R&D Systems Inc., 614 McKinley Place Nebr., Minneapolis, Minn. 55413, USA) are added to column 12 of the MSD plate to generate a standard calibration curve (about 0 to 30000 pg/ml final).

For the Whole Blood assay, plates are washed after 2 hours shaking with a Skanwasher 300 version B (Skatron Instruments AS. PO Box 8, N-3401 Lier, Norway). About 40 μl (ul) of diluted sulfo-TAG antibody (ca. 1 μg/ml final) is added, the plates are shaken at room temperature for 1 hours, and the plates washed again as above. About 150 μl (ul) of Read Buffer T (2×) is added to the plates, which are then read on a MSD Sector 6000.

For the PBMC assay, about 20 μl (ul) of diluted sulfo-TAG antibody (ca. 1 μg/ml final) is added to each well, and the plates/wells are shaken at room temperature for 2 hours. Finally, about 90 μl (ul) of MSD Read Buffer P (diluted to 2.5 times with distilled water) is added and the plates are read on a MSD Sector 6000.

### Data Analysis

Data analysis is performed with ActivityBase/XC50 module (ID Business Solutions Ltd., 2 Occam Court, Surrey Research Park, Guildford, Surrey, GU2 7QB UK). Data are normalized and expressed as % inhibition using the formula 100*((U−C1)/(C2−C1)) where U is the unknown value, C1 is the average of the high signal (0%) control wells (column 10), and C2 is the average of the low signal (100%) control wells (column 11). Curve fitting is performed with the following equation: y=A+((B−A)/(1+(10̂x/10̂C)̂D)), where A is the minimum response, B is the maximum response, C is the log10(IC50), and D is the Hill slope. The results for each compound are recorded as pIC50 values (—C in the above equation).

### TNF-α (TNF-alpha) IGEN Assay

Ca. 50 μl supernatant from either whole blood or PBMC assay plates is transferred to a 96 well polypropylene plate. Each plate also contains a TNF-α standard curve (ca. 0 to 30000 pg/ml: R+D Systems, 210-TA). Ca. 50 μl (ul) of streptavidin/biotinylated anti-TNF-α antibody mix, ca. 25 μl ruthenium tagged anti-TNF-α monoclonal and ca. 100 μl PBS containing 0.1% bovine serum albumin are added to each well and the plates are sealed and shaken for ca. 2 hours before being read on an IGEN instrument.

### TNF-α (TNF-alpha) ELISA Assay (Enzyme Linked Immunosorbant Assay)

Human TNF-α can be assayed using a commercial ELISA assay kit (AMS Biotechnology, 211-90-164-40) according to the manufacturers' instructions but with TNF-α calibration curves prepared using Pharmingen TNF-α (cat No. 555212).

### In Vivo Biological Assays

The in vitro enzymatic PDE4B inhibition assay(s) described above or generally similar or generally analogous assays should be regarded as being the primary test(s) of biological activity. However, some additional in vivo biological tests, which are optional only, and which are not an essential measure of activity, efficacy or side-effects, and which have not necessarily been carried out, are described below.

### In Vivo Assay A:

### Activity of Topically-Applied Compounds in a Pig Model of Atopic Dermatitis: Effect of Compounds, Applied by Skin Topical Administration, on the Dinitrofluorobenzene (DNFB)-Induced Delayed Type Hypersensitivity (DTH) Response in Pigs

### General Study Design:

The pig DTH (delayed type hypersensitivity) model of contact hypersensitivity utilizes the Th2-mediated inflammatory response in pig skin to mimic the pathology of atopic dermatitis in humans. The model measures the potential anti-inflammatory effect of compounds, topically-applied to the skin, on the acute DTH (delayed type hypersensitivity) response in castrated male Yorkshire pigs.

In general in the assay, pigs (domestic Yorkshire pigs, 15-18 kg at time of sensitization, castrated males) are sensitized by topical application of ca. 10% (w/v) dinitrofluorobenzene (DNFB) dissolved in DMSO:acetone:olive oil (ca. 1:5:3) (ca. 40 mg DNFB, 400 microlitre solution total) to the ears (outer) and groin (inner) on day −12 and −5. The pigs are then challenged with ca. 0.6% (w/v) DNFB applied to randomized sites on the shaved back of the pigs (ca. 90 micrograms/site; sites are identified and numbered by grid made with marking pen).

On the day of challenge, the treatments are performed at the challenge sites at about 2 hours prior to and about 6 hours after challenge (for DMSO/acetone solutions/suspensions containing the PDE4 inhibitor, to maximize exposure to drug), or at about 30 minutes after and about 6 hours after challenge (for topical ointments or creams containing the PDE4 inhibitor, representing a more clinically relevant treatment protocol).

After 24 hrs and 48 hrs post challenge, test sites are visually evaluated for intensity and extent of erythema by measuring the diameter of the reaction at its widest point and assigning scores of 0 to 4 for each of erythema intensity and erythema extent. Induration (a measure of swelling) is also scored 0 to 4. Scores for erythema intensity, erythema extent and induration are assigned according to the following criteria: Intensity of Erythema: O-normal, 1=minimal, barely visible, 2=mild, 3=moderate, 4=severe. Extent of Erythema (not raised): 0=no edema, 1=macules of pin head size, 2=lentil sized macules, 3=confluent macules, 4=diffuse over entire site. Induration (palpable): 0=normal, 1=nodules of pin head size, 2=doughy lentil sized nodules, 3=confluent firm nodules, 4=diffuse hard lesion. The summed visual score at ca. 24 and 48 hours includes the individual scores for erythema intensity, erythema extent, and induration; so the maximal summed score for each site would be 12. High summed scores can generally indicate a high inflammatory response. Visual scores are subject to some inaccuracy/error.

Differences in the summed score between adjacent control (placebo) and treatment sites on the grids are calculated (see experimental design for details). This difference value is then used to determine the percent inhibition compared to the summed score for the control (placebo) sites. The more negative the difference value, the greater the calculated inhibition. Percent inhibition of (percent inhibition compared to) the mean summed score can be calculated.

About 24 and 48 hours after challenge, treatment sites can optionally also be visually evaluated for lesion area.

### Experimental Design

An 8 row by 3 column grid is outlined on the back of each pig. Due to potential heterogeneity in skin thickness, etc, across various regions of the back, the assignment of treatments and their controls were assigned to each row/column in a systematic manner. Specifically, each row contained a treatment and its control (n=1 or 2 of each in the 3 columns). Row assignments were randomized across pigs to ensure that treatments were not always in the same region of the pig's backs. Column assignments were also randomized: the number of treatments per row (either 1 or 2) was randomly chosen, and treatment and control assignments were randomized across the 3 columns.

### Data Analysis

Proper statistical analysis will incorporate features of the experimental design, which is generically known as a randomized block design (a block being a row on the grid). For each row, the average treatment and control response is calculated. The difference, d, between treatment and control is determined, and this is the response variable. The set of differences for each treatment and pig is analyzed using a 2 factor analysis of variance (ANOVA), with pig and treatment as factors. The average difference of each treatment group is tested against 0 using results from the two factor ANOVA; p-values<0.05 are considered significant. (However, given the number of tests, a significant p-value cutoff of <0.01 might optionally (but not necessarily) be used to protect against possible false positive results.)

Statistical analysis which does not incorporate features of the randomized block design generally will not be as powerful (i.e. will tend to generate higher p-values). For example, pooling treatment and control data across all rows and columns from all pigs and conducting t-tests on treatment vs. control will be expected to have larger variance estimates because heterogeneity within and between pigs are part of the variance. In the 2 factor ANOVA on the differences, within and between effects are factored out of the variance resulting in more powerful tests. See Statistics for Experimenters, Box, Hunter and Hunter, 2^{nd} edition, chapter 4 (Wiley and Sons, 2005).

Data are presented as Change (average difference in Matched pair scores) and p value (2 factor ANOVA via SAS software).

### Results: Pig DTH Assay—Study #8

A single specific ointment composition, being either Composition Example 1 or Composition Example 3 (Ointment D or D2) as disclosed hereinafter, and containing 2% w/w of the compound of the invention, and white petrolatum and ST-Cyclomethicone 5-NF™, significantly (p<0.05) inhibited inflammation scores at the 48 hour time point, but not at the 24 hour time point (placebo/control=Reference Composition Example 2 or 4 Placebo Ointment AP or AP2, disclosed hereinafter). See the results tabulated below under the label “Modified White Petrolatum Ointment”.

A single specific cream composition, being either Composition Example 5 or Composition Example 7 (Water-in-oil cream Cr-D or Cr-D2) as disclosed hereinafter, and containing inter alia propylene glycol and 2% w/w of the compound of the invention, significantly (p<0.05) inhibited inflammation scores at the 48 hour time point, but not at the 24 hour time point (placebo/control=Reference Composition Example 6 or 8=Placebo Water-in-oil cream Cr-AP or Cr-AP2, disclosed hereinafter). See the results tabulated below under the label “Cream”.

| | | | |
|---|---|---|---|
| | | | p |
| Drug % | Change | study | value |

24 hr
Modified White
Petrolatum Ointment

| | | | |
|---|---|---|---|
| 2 | 0.15 | 8 | 0.766 |

24 hr
Cream

| | | | |
|---|---|---|---|
| 2 | 0.10 | 8 | 0.843 |

| | | | |
|---|---|---|---|
| | | | p |
| Drug % | Change | study | value |

48 hr
Modified White
Petrolatum Ointment

| | | | |
|---|---|---|---|
| 2 | −1.10 | 8 | 0.031 |

48 hr
Cream

| | | | |
|---|---|---|---|
| 2 | −1.10 | 8 | 0.031 |

### Results: Pig DTH Assay—Study #9

A single specific ointment composition, being either Composition Example 3 or Composition Example 1 (Ointment D2 or D) as disclosed hereinafter, and containing 2% w/w of the compound of the invention, and white petrolatum and ST-Cyclomethicone 5-NF™, significantly (p<0.05) inhibited inflammation scores at the 24 and 48 hour time point (placebo/control=Reference Composition Example 4 or 2=Placebo Ointment AP2 or AP, disclosed hereinafter). See the results tabulated below under the label “Modified White Petrolatum Ointment”.

A single specific cream composition, being either Composition Example 7 or Composition Example 5 (Water-in-oil cream Cr-D2 or Cr-D) as disclosed hereinafter, and containing inter alia propylene glycol and 2% w/w of the compound of the invention, appeared to be somewhat inhibitory (negative Change observed), but the differences (with p≧0.05) did not reach statistical significance (placebo/control=Reference Composition Example 8 or 6=Placebo Water-in-oil cream Cr-AP2 or Cr-AP, disclosed hereinafter). See the results tabulated below under the label “Cream”.

| | | | |
|---|---|---|---|
| | | | p |
| Drug % | Change | study | value |

24 hr
Modified White
Petrolatum Ointment

| | | | |
|---|---|---|---|
| 2 | −1.29 | 9 | 0.028 |

24 hr
Cream

| | | | |
|---|---|---|---|
| 2 | −1.00 | 9 | 0.086 |

| | | | |
|---|---|---|---|
| | | | p |
| Drug % | Change | study | value |

48 hr
Modified White
Petrolatum Ointment

| | | | |
|---|---|---|---|
| 2 | −1.92 | 9 | about |
| | | | 0.000 |

48 hr
Cream

| | | | |
|---|---|---|---|
| 2 | −0.65 | 9 | 0.156 |

Compounds or pharmaceutical compositions showing apparent activity in the above pig DTH model (In Vivo Assay A) may have potential utility via skin topical administration in the treatment and/or prophylaxis of atopic dermatitis, e.g. in humans. The preliminary nature of In Vivo Assay A, and the potential for error or for variation in results when the assay is repeated (see e.g. results of Study #8 cf. those of Study #9) is noted.

### In Vivo Assay 1. LPS-Induced Pulmonary Neutrophilia in Rats: Effect of Orally Administered PDE4 Inhibitors

Pulmonary neutrophil influx is thought to be a significant component to the family of pulmonary diseases like chronic obstructive pulmonary disease (COPD) which can involve chronic bronchitis and/or emphysema (G. F. Filley, Chest 2000; 117(5); 251s-260s). The purpose of this neutrophilia model is to study the potentially anti-inflammatory effects in vivo of orally administered PDE4 inhibitors on neutrophilia induced by inhalation of aerosolized lipopolysaccharide (LPS), modelling the neutrophil inflammatory component(s) of COPD. See the literature section below for scientific background.

Male Lewis rats (Charles River, Raleigh, N.C., USA) weighing approximately 300-400 grams are pretreated with either (a) test compound, for example suspended in about 0.5% methylcellulose (obtainable from Sigma-Aldrich, St Louis, Mo., USA) in water or (b) vehicle only, delivered orally in a dose volume of ca. 10 ml/kg. Generally, dose response curves can for example be generated using the following approx. doses of PDE4 inhibitors: 2.0, 0.4, 0.08, 0.016 and 0.0032 mg/kg (or alternatively approx. 10, 2.0, 0.4, 0.08 and 0.016 mg/kg). About thirty minutes following pretreatment, the rats are exposed to aerosolized LPS (Serotype E. Coli 026:B6 prepared by trichloroacetic acid extraction, obtainable from Sigma-Aldrich, St Louis, Mo., USA), generated from a nebulizer containing a ca. 100 μg/ml LPS solution (ca. 100 ug/ml). Rats are exposed to the LPS aerosol at a rate of ca. 4 L/min for ca. 20 minutes. LPS exposure is carried out in a closed chamber with internal dimensions of roughly 45 cm length×24 cm width×20 cm height. The nebulizer and exposure chamber are contained in a certified fume hood. At about 4 hours-post LPS exposure the rats are euthanized by overdose with pentobarbital at ca. 90 mg/kg, administered intraperitoneally. Bronchoalveolar lavage (BAL) is performed through a 14 gauge blunt needle into the exposed trachea. Five, 5 ml washes are performed to collect a total of 25 ml of BAL fluid. Total cell counts and leukocyte differentials are performed on BAL fluid in order to calculate neutrophil influx into the lung. Percent neutrophil inhibition at each dose (cf. vehicle) is calculated and a variable slope, sigmoidal dose-response curve is generated, usually using Prism Graph-Pad. The dose-response curve is used to calculate an ED50 value (in mg per kg of body weight) for inhibition by the PDE4 inhibitor of the LPS-induced neutrophilia.

Alternative method: In an alternative simpler embodiment of the procedure, a single oral dose of 10 mg/kg, or more usually 1.0 mg/kg or 0.3 mg/kg, of the PDE4 inhibitor (or vehicle) is administered to the rats, and percent neutrophil inhibition is calculated and reported for that specific dose.

Literature:

Filley G. F. Comparison of the structural and inflammatory features of COPD and asthma. Chest. 2000; 117(5) 251s-260s.

Howell R E, Jenkins L P, Fielding L E, and Grimes D. Inhibition of antigen-induced pulmonary eosinophilia and neutrophilia by selective inhibitors of phosphodiesterase types 3 and 4 in brown Norway rats. Pulmonary Pharmacology. 1995; 8: 83-89.

Spond J, Chapman R, Fine J, Jones H, Kreutner W, Kung T T, Minnicozzi M. Comparison of PDE 4 inhibitors, Rolipram and SB 207499 (Ariflo™), in a rat model of pulmonary neutrophilia. Pulmonary Pharmacology and Therapeutics. 2001; 14: 157-164.

Underwood D C, Osborn R R, Bochnowicz S, Webb E F, Rieman D J, Lee J C, Romanic A M, Adams J L, Hay D W P, and Griswold D E. SB 239063, a p38 MAPK inhibitor, reduces neutrophilia, inflammatory cytokines, MMP-9, and fibrosis in lung. Am J Physiol Lung Cell Mol Physiol. 2000; 279: L895-L902.

### In Vivo Assay 2. Rat Pica Model of Emesis

Background: Selective PDE4 inhibitors are thought to inhibit inflammation in various in vitro and in vivo models by increasing intracellular levels of cAMP of many immune cells (e.g. lymphocytes, monocytes). However, a side effect of some PDE4 inhibitors in some species is emesis. Because many rat models of inflammation are well characterized, they can be used in procedures (see e.g. In Vivo Assay 1 above) to show beneficial anti-inflammatory effects of PDE 4 inhibitors. However rats have no emetic response (they have no vomit reflex), so that the relationship between beneficial anti-inflammatory effects of PDE 4 inhibitors and emesis is difficult to study directly in rats.

However, in 1991, Takeda et al. (see Literature section below) demonstrated that the pica feeding response is analogous to emesis in rats. Pica feeding is a behavioural response to illness in rats wherein rats eat non-nutritive substances such as earth or in particular clay (e.g. kaolin) which may help to absorb toxins. Pica feeding can be induced by motion and chemicals (especially chemicals which are emetic in humans), and can be inhibited pharmacologically with drugs that inhibit emesis in humans. The Rat Pica Model, In Vivo Assay 2, can determine the level of pica response of rats to PDE 4 inhibition at pharmacologically relevant doses in parallel to in vivo anti-inflammatory Assays in (a separate set of) rats (e.g. In Vivo Assay 1 above).

Anti-inflammatory and pica assays in the same species together can provide data on the “therapeutic index” (TI) in the rat of the compounds/salts of the invention. The Rat TI can for example be calculated as the ratio of a) the potentially-emetic Pica Response ED50 dose from Assay 2 to b) the rat anti-inflammatory ED50 dose (e.g. measured by rat neutrophilia-inhibition in eg In Vivo Assay 1), with larger TI ratios possibly indicating lower emesis at many anti-inflammatory doses. This might allow a choice of a non-emetic or low-emetic pharmaceutical dose of the compounds or salts of the invention which has an anti-inflammatory effect. It is recognised however that achieving a low-emetic PDE4 inhibitory compound is not essential to the invention.

Procedure: On the first day of the experiment, the rats are housed individually in cages without bedding or “enrichment”. The rats are kept off of the cage floor by a wire screen. Pre-weighed food cups containing standard rat chow and clay pellets are placed in the cage. The clay pellets, obtainable from Languna Clay Co, City of Industry, Calif., USA, are the same size and shape as the food pellets. The rats are acclimated to the clay for 72 hours, during which time the cups and food and clay debris from the cage are weighed daily on an electronic balance capable of measuring to the nearest 0.1 grams. By the end of the 72 hour acclimation period the rats generally show no interest in the clay pellets.

At the end of 72 hours the rats are placed in clean cages and the food cups weighed. Rats that are still consuming clay regularly are removed from the study. Immediately prior to the dark cycle (the time when the animals are active and should be eating) the animals are split into treatment groups and dosed orally with a dose of the compound or salt of the invention (different doses for different treatment groups) or with vehicle alone, at a dose volume of ca. 2 ml/kg. In this oral dosing, the compound or salt can for example be in the form of a suspension in about 0.5% methylcellulose (obtainable Sigma-Aldrich, St. Louis, Mo., USA) in water. The food and clay cups and cage debris are weighed the following day and the total clay and food consumed that night by each individual animal is calculated.

A dose response is calculated by first converting the data into quantal response, where animals are either positive or negative for the pica response. A rat is “pica positive” if it consumes greater than or equal to 0.3 grams of clay over the mean of its control group. The D50 value is usually calculated using logistic regression performed by the Statistica software statistical package. A Pica Response ED50 value in mg per kg of body weight can then be calculated.

The Pica Response ED50 value can be compared to the neutrophilia-inhibition ED50 values for the same compound administered orally to the rat (measurable by In Vivo Assay 1 above), so that a Therapeutic Index (TI) in rats can be calculated thus:

$$Rat Therapeutic index ( T I ) ( 50 / 50 ) = \frac{Pica Response ED 50 value}{rat neutrophilia \text{-} inhibition ED 50 value}$$

In general, the Therapeutic Index (TI) calculated this way can be substantially different to, and for example (without being bound) can be substantially higher than, the TI (D20O/D50) calculated in the ferret (see In vivo Assay 3 below).

Alternatively, e.g. for a simpler test, the In Vivo Assay 2 (pica) can use only a single oral dose of the test compound (e.g. 10 mg/kg orally).

Literature:

Beavo J A, Contini, M., Heaslip, R. J. Multiple cyclic nucleotide phosphodiesterases. Mol Pharmacol. 1994; 46:399-405.

Spond J, Chapman R, Fine J, Jones H, Kreutner W, Kung T T, Minnicozzi M. Comparison of PDE 4 inhibitors, Rolipram and SB 207499 (Ariflo™), in a rat model of pulmonary neutrophilia. Pulmonary Pharmacology and Therapeudtics. 2001; 14:157-164.

Takeda N, Hasegawa S, Morita M, and Matsunaga T. Pica in rats is analogous to emesis: an animal model in emesis research. Pharmacology, Biochemistry and Behavior. 1991; 45:817-821.

Takeda N, Hasegawa S, Morita M, Horii A, Uno A, Yamatodani A and Matsunaga T. Neuropharmacological mechanisms of emesis. I. Effects of antiemetic drugs on motion- and apomorphine-induced pica in rats. Meth Find Exp Clin Pharmacol. 1995; 17(9) 589-596.

Takeda N, Hasegawa S, Morita M, Horii A, Uno A, Yamatodani A and Matsunaga T. Neuropharmacological mechanisms of emesis. I. Effects of antiemetic drugs on cisplatin-induced pica in rats. Meth Find Exp Clin Pharmacol. 1995; 17(9) 647-652.

### In Vivo Assay 3. Evaluation of Therapeutic Index of Orally-administered PDE 4 Inhibitors in the Conscious Ferret

### 1.1 Materials

The following materials can be used for these studies:

PDE4 inhibitors are prepared for oral (p.o.) administration by dissolving in a fixed volume (ca. 1 ml) of acetone and then adding cremophor to ca. 20% of the final volume. Acetone is evaporated by directing a flow of nitrogen gas onto the solution. Once the acetone is removed, the solution is made up to final volume with distilled water. LPS is dissolved in phosphate buffered saline.

### 1.2 Animals

Male ferrets (Mustela Pulorius Furo, weighing 1-2 kg) are transported and allowed to acclimatise for not less than 7 days. The diet comprises SDS diet C pelleted food given ad lib with Whiskers™ cat food given 3 times per week. The animals are supplied with pasteurised animal grade drinking water changed daily.

### 1.3 Experimental Protocol(s)

1.3.1 Dosing with PDE4 Inhibitors

PDE4 inhibitors are administered orally (p.o.), using a dose volume of ca. 1 ml/kg. Ferrets are fasted overnight but allowed free access to water. The animals are orally dosed with vehicle or PDE 4 inhibitor using a ca. 15 cm dosing needle that is passed down the back of the throat into the oesophagus. After dosing, the animals are returned to holding cages fitted with perspex doors to allow observation, and given free access to water. The animals are constantly observed and any emetic episodes (retching and vomiting) or behavioural changes are recorded. The animals are allowed access to food ca. 60-90 minutes after p.o. dosing.

### 1.3.2 Exposure to LPS

About thirty minutes after oral dosing with compound or vehicle control, the ferrets are placed into sealed perspex containers and exposed to an aerosol of LPS (ca. 30 μg/ml=ca. 30 ug/ml) for ca. 10 minutes. Aerosols of LPS are generated by a nebuliser (DeVilbiss, USA) and this is directed into the perspex exposure chamber. Following a 10-minute exposure period, the animals are returned to the holding cages and allowed free access to water, and at a later stage, food. General observation of the animals continues for a period of at least 2.5 hours post oral dosing. All emetic episodes and behavioural changes are recorded.

### 1.3.3 Bronchoalveolar Lavage and Cell Counts

About six hours after LPS exposure the animals are killed by overdose of sodium pentobarbitone administered intraperitoneally. The trachea is then cannulated with polypropylene tubing and the lungs lavaged twice with ca. 20 ml heparinised (10 units/ml) phosphate buffered saline (PBS). The bronchoalveolar lavage (BAL) samples are centrifuged at ca. 1300 rpm for ca. 7 minutes. The supernatant is removed and the resulting cell pellet re-suspended in ca. 1 ml PBS. A cell smear of re-suspended fluid is prepared and stained with Leishmans stain to allow differential cell counting. A total cell count is made using the remaining re-suspended sample. From this, the total number of neutrophils in the BAL sample is determined.

### 1.3.4 Pharmacodynamic Readouts

The following parameters are recorded:

a) % inhibition of LPS-induced pulmonary neutrophilia to determine the dose of PDE4 inhibitor which gives 50% inhibition (D50).
b) Emetic episodes—the number of vomits and retches are counted to determine the dose of PDE4 inhibitor that gives a 20% incidence of emesis (D20).
c) A therapeutic index (TI), using this assay, is then calculated for each PDE4 inhibitor using the following equation:

$$Ferret Therapeutic index ( T I ) ( D 20 / D 50 ) = \frac{D 20 incidence of emesis in ferret}{D 50 inhibition of neutrophilia in ferret}$$

It is noted that the Ferret Therapeutic index (TI) (D₂O/D50) calculated using this in vivo Assay 3 can be substantially different to, and for example (without being bound) can be substantially lower than, the Rat TI (50/50) calculated using the rat oral inflammation and pica feeding Assays 1+2.

All publications, including but not limited to patents and patent applications, cited in this specification are herein incorporated by reference as if each individual publication were specifically and individually indicated to be incorporated by reference herein as though fully set forth.

### EXAMPLES

The various aspects of the invention will now be described by reference to the following examples. These examples are merely illustrative and are not to be construed as a limitation of the scope of the present invention.

In this section, “Intermediates” can represent syntheses of intermediate compounds intended for use in the synthesis of one or more of the “Examples”, and/or “Intermediates” can represent syntheses of intermediate compounds which can be used in the synthesis of compounds of formula (I) or salts thereof. “Examples” are examples of a compound or salt of the invention, i.e. examples of a compound of formula (I) or a salt thereof.

### Abbreviations Used Herein

AcOH acetic acid
Ac₂O acetic anhydride
BEMP 2-t-butylimino-2-diethylamino-1,3-dimethylperhydro-1,3,2-diazaphosphazine
BOC₂O di tert-butyl carbonate
DMSO dimethyl sulfoxide
DCM dichloromethane
DMF dimethyl formamide
DIPEA diisopropylethyl amine (ⁱPr₂NEt)
EDC 1-(3-Dimethylaminopropyl)-3-ethylcarbodiimide hydrochloride
EtOAc ethyl acetate
Et₂O diethyl ether
Et₃N triethylamine
EtOH ethanol
HATU O-(7-Azabenzotriazol-1-yl)-N,N,N′,N′-tetramethyluronium hexafluorophosphate
HBTU O-(Benzotriazol-1-yl)-N,N,N′,N′-tetramethyluronium hexafluorophosphate
HOBT hydroxybenzotriazole=1-hydroxybenzotriazole
Lawesson's reagent 2,4-bis(4-methoxyphenyl)-1,3-dithia-2,4-diphosphetane-2,4-disulphide
MeCN acetonitrile
MeOH methanol
PyBOP Benzotriazole-1-yl-oxy-trispyrrolidinophosphonium hexafluorophosphate
THF Tetrahydrofuran
TFA Trifluoroacetic acid
Tᵢₙₜ internal temperature of the reaction mixture
HPLC high performance liquid chromatography
h hours
min minutes
LCMS liquid chromatography/mass spectroscopy
NMR nuclear magnetic resonance (in which: s=singlet, d=doublet, t=triplet, q=quartet, dd=doublet of doublets, m=multiplet, H=no. of protons)
SPE solid phase extraction
TLC thin layer chromatography
T_{RET} retention time (e.g. from LCMS)
Room temperature (ambient temperature): this is usually in the range of about 18 to about 25° C.

### General Experimental Details

### Machine Methods Used Herein:

### LCMS (Liquid Chromatography/Mass Spectroscopy)

Waters ZQ mass spectrometer operating in positive ion electrospray mode, mass range 100-1000 amu.
UV wavelength: 215-330 nm
Column: 3.3 cm×4.6 mm ID (internal diameter), 3 μm (3 micrometres) ABZ+PLUS
Flow Rate: 3 ml/min
Injection Volume: 5 μl (5 microlitres)
Solvent A: 0.05% v/v solution of formic acid in a mixture of [95% acetonitrile and 5% water]
Solvent B: aqueous solution of [0.1% v/v formic acid+10 mMolar ammonium acetate]
Gradient: Mixtures of Solvent A and Solvent B are used according to the following gradient profiles (expressed as % Solvent A in the mixture): 0% A/0.7 min, 0-100% A/3.5 min, 100% A/1.1 min, 100-0% A/0.2 min

It should be noted that retention times (T_{RET}) quoted herein are inherently variable (e.g. the variability can be about +/−0.2 min or more.). Variability can arise e.g. when samples are run on different Waters machines, or on the same Waters machine at different times of day or under slightly different conditions, even when the same type of column and identical flow rates, injection volumes, solvents and gradients are used.

### Mass Directed Autoprep HPLC

The preparative HPLC column generally used is a Supelcosil ABZplus (10 cm×2.12 cm internal diameter; particle size 5 μm=5 micrometres) e.g. for eluent containing formic acid, or is a C18 column (10 cm×2.12 cm internal diameter; particle size 5 μm=5 micrometres) e.g. for eluent containing trifluoroacetic acid. A mass spectrometer attached to the end of the column can detect peaks arising from eluted compounds.

UV wavelength: usually 200-320 nm
Flow: 20 ml/min

### Injection Volume: 0.5 to 1 ml

Gradient systems: mixtures of Solvent A and Solvent B are used according to a choice of 5 generic gradient profiles (expressed as % Solvent B in the mixture), ranging from a start of 0 to 50% Solvent B, with all finishing at 100% Solvent B to ensure total elution. Generally, two alternative solvent systems have been used, Method 1 and Method 2:

### Method 1

Solvent A: 0.1% v/v aqueous formic acid solution
Solvent B: 0.05% v/v solution of formic acid in a mixture of [95% acetonitrile and 5% water]

It is thought that compounds isolated by this method can sometimes be isolated as formate salts.

### Method 2

Solvent A: 0.1% v/v aqueous trifluoroacetic acid solution
Solvent B: solution of 0.1% v/v trifluoroacetic acid in acetonitrile

It is thought that compounds isolated by this method can sometimes be isolated as trifluoroacetate salts.

### Intermediates and Examples

Reagents not detailed in the text below are usually commercially available from chemicals suppliers, e.g. established suppliers such as Sigma-Aldrich. The addresses and/or contact details of the suppliers for some of the starting materials mentioned in the Intermediates and Examples below or the Assays above, or suppliers of miscellaneous chemicals in general, are as follows:
Albemarle Corporation, 451 Florida Street, Baton Rouge, La. 70801, USA; or Albemarle Europe SPRL, Parc Scientific de LLN, Rue du Bosquet 9, B-1348 Louvain-la-Neuve, Belgium
Acros Organics, A Division of Fisher Scientific Company, 500 American Road, Morris Plains, N.J. 07950, USA
Apin Chemicals Ltd., 82 C Milton Park, Abingdon, Oxon OX14 4RY, United Kingdom
Aldrich (catalogue name), Sigma-Aldrich Company Ltd., Dorset, United Kingdom, telephone: +44 1202 733114; Fax: +44 1202 715460; ukcusts@eurnotes.sial.com; or
Aldrich (catalogue name), Sigma-Aldrich Corp., P.O. Box 14508, St. Louis, Mo. 63178-9916, USA; telephone: +1-314-771-5765; fax: +1-314-771-5757; custserv@sial.com; or
Aldrich (catalogue name), Sigma-Aldrich Chemie GmbH, Munich, Germany; telephone: +49 89 6513 0; Fax: +49 89 6513 1169; deorders@eurnotes.sial.com.
Amersham Biosciences UK Ltd, Pollards Wood, Chalfont St Giles, Buckinghamshire HP8 4SP, United Kingdom
Art-Chem GmbH: (a) Lininsky prospect 47, Moscow 119991, Russia, or (b) Campus Berlin-Buch, Haus B55, Robert-Roessle Strasse 10, 13125 Berlin, Germany
AstaTech, Inc., 8301 Torresdale Ave., 19C, Philadelphia, Pa. 19136, USA
Bayer A G, Business Group Basic and Fine Chemicals, D-51368 Leverkusen, Germany
Chemical Building Blocks (catalogue name), Ambinter, 46 quai Louis Bleriot, Paris, F-75016, France
Combi-Blocks Inc., 7949 Silverton Avenue, Suite 915, San Diego, Calif. 92126, USA
Enamine, 23 A. Motrosova Street, Kiev 01103, Ukraine
Fluka Chemie AG, Industriestrasse 25, P.O. Box 260, CH-9471 Buchs, Switzerland
Fluorochem Ltd., Wesley Street, Old Glossop, Derbyshire SK13 7RY, United Kingdom
Interchim Intermediates (catalogue name), Interchim, 213 Avenue Kennedy, BP 1140, Montlucon, Cedex, 03103, France
Lancaster Synthesis Ltd., Newgate, White Lund, Morecambe, Lancashire LA3 3DY, United Kingdom
Matrix Scientific, P.O. Box 25067, Columbia, S.C. 29224-5067, USA
MicroChemistry Building Blocks (catalogue name), MicroChemistry-RadaPharma, Shosse Entusiastov 56, Moscow, 111123, Russia
Molecular Devices Corporation, Sunnydale, Calif., USA
Oakwood Products Inc., 1741, Old Dunbar Road, West Columbia, S.C., 29172, USA
Peakdale Molecular Ltd., Peakdale Science Park, Sheffield Road, Chapel-en-le-Frith, High Peak SK23 OPG, United Kingdom
SALOR (catalogue name) (Sigma Aldrich Library of Rare Chemicals), Aldrich Chemical Company Inc, 1001 West Saint Paul Avenue, Milwaukee, Wis. 53233, USA
Sigma (catalogue name), Sigma-Aldrich Corp., P.O. Box 14508, St. Louis, Mo. 63178-9916, USA; see “Aldrich” above for other non-US addresses and other contact details
SIGMA-RBI, One Strathmore Road, Natick, Mass. 01760-1312, USA
TimTec Corporation (e.g. “TimTec Overseas Stock”), 100 Interchange Boulevard, Newark, Del. 19711, USA
“TimTec Building Blocks A or B”, or “TimTec Stock Library”, TimTec, Inc., P 0 Box 8941, Newark, Del. 19714-8941, USA

### Intermediates

### Intermediate 1 Ethyl 4-chloro-1,6-diethyl-1H-pyrazolo[3,4-b]pyridine-5-carboxylate

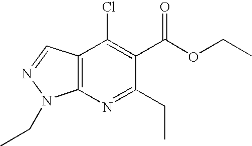

Diethyl propanoylpropanedioate which is Et-C(O)—C(CO₂Et)₂ [e.g. see J. Org. Chem., (1976), 41 (24), 3857] (2.74 g) was dissolved in phosphoryl chloride (30 ml), tributylamine (3 ml) was added cautiously, and the mixture was heated to 110° C. for 5 h. The mixture was concentrated under reduced pressure to remove the phosphoryl chloride, and the residue was dissolved in diethyl ether (about 20 ml) and n-hexane added until two layers formed. The top layer (diethyl ether) was collected and the diethyl ether/hexane extraction procedure repeated twice more. The combined ether extracts were washed with 1N HCl solution (2×50 ml), 1N sodium hydroxide solution and water, and were dried and evaporated to give diethyl (1-chloropropylidene)propanedioate (1.61 g). LCMS showed MH⁺=235; T_{RET}=3.21 min.

A mixture of diethyl (1-chloropropylidene)propanedioate (1.61 g) and 1-ethyl-1H-pyrazol-5-amine [e.g. available from Aldrich Chemical Company Inc., Albemarle Corporation, Art-Chem GmbH, Enamine and/or TimTec Corporation] (0.755 g) in toluene (40 ml) was treated with triethylamine (1.8 ml) and the mixture heated at 120° C. for 16 h. The solvent was evaporated under reduced pressure and the residue was dissolved in phosphoryl chloride (50 ml) and heated at 110° C. overnight. The mixture was evaporated under reduced pressure to remove the phosphoryl chloride, and the residue was partitioned between ethyl acetate and saturated aqueous sodium hydrogen carbonate solution. The organic layer was separated, dried (sodium sulphate) and evaporated under reduced pressure. The residue was purified on an SPE cartridge (silica, 50 g) eluting with 5% ethyl acetate in cyclohexane to give the title compound as a yellow oil (1.22 g). LCMS showed MH⁺=282; T_{RET}=3.40 min.

### Intermediate 1 (Alternative Synthesis B) Ethyl 4-chloro-1,6-diethyl-1H-pyrazolo[3,4-b]pyridine-5-carboxylate

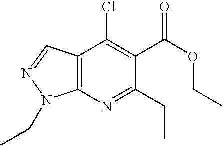

Triethylamine (230 ml) is added dropwise to a mixture of diethyl (1-chloropropylidene)propanedioate (208 g) and 1-ethyl-1H-pyrazol-5-amine (101 g) in toluene (2.65 L). The mixture is heated under reflux for 16 hours. The reaction mixture is cooled to room temperature, and the solid removed by filtration. The filtrate is evaporated under reduced pressure. The residue is heated under reflux in phosphorus oxychloride (POCl₃, 2.65 L) for 16 hrs. Excess phosphorus oxychloride is removed under reduced pressure and the cooled mixture is poured onto a mixture of saturated aqueous NaHCO₃ solution (4 L) and EtOAc (1.5 L). The organic layer is separated and the aqueous layer further extracted with ethyl acetate (2×1 L). The combined EtOAc extracts are washed with saturated aqueous NaHCO₃ solution (2×2 L) and dried (Na₂SO₄). Evaporation of solvent under reduced pressure affords the crude product. The crude product is purified by chromatography (silica gel, 60-120 mesh, 3.5 kg), eluting with 3% EtOAc in hexane. Fractions containing the product are pooled and evaporated to give the title compound.

### Intermediate 2 Ethyl 1,6-diethyl-4-(tetrahydro-2H-pyran-4-ylamino)-1H-pyrazolo[3,4-b]pyridine-5-carboxylate

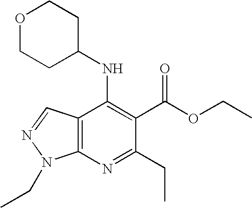

Ethyl 4-chloro-1,6-diethyl-1H-pyrazolo[3,4-b]pyridine-5-carboxylate (0.50 g) (e.g. this can be as prepared in Intermediate 1) was dissolved in 1-methyl-2-pyrrolidinone (5 ml) and treated with tetrahydro-2H-pyran-4-amine hydrochloride (0.49 g) [e.g. this can be as prepared in Intermediate 12, see below] and DIPEA (0.60 ml) at 120° C. overnight. The mixture was allowed to cool and was partitioned between ethyl acetate (3×50 ml) and water (50 ml). The organic layer was separated, dried and evaporated in vacuo. The residue was purified on an SPE cartridge (e.g. solid phase can be ca. 20 g or ca. 50 g; e.g. can be silica) eluting with from 5% to 20% ethyl acetate in cyclohexane to give the title compound (0.413 g). LCMS showed MH⁺=347; T_{RET}=3.05 min.

### Intermediate 2 (Alternative Synthesis B) Ethyl 1,6-diethyl-4-(tetrahydro-2H-pyran-4-ylamino)-1H-pyrazolo[3,4-b]pyridine-5-carboxylate

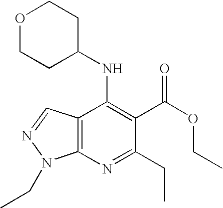

To a solution of ethyl 4-chloro-1,6-diethyl-1H-pyrazolo[3,4-b]pyridine-5-carboxylate (36 g, 127.8 mmol) (which can e.g. be as prepared in Intermediate 1) in 1-methyl-2-pyrrolidinone (300 ml) is added DIPEA (44.5 ml, 255.6 mmol). Tetrahydro-2H-pyran-4-amine (e.g. available from Peakdale and/or Combi-Blocks Inc., 15.5 g, 153.3 mmol) is added and the reaction mixture is heated at 115° C. with stirring overnight. The cooled mixture is poured into water (1200 ml), which may form an oily mixture. This is extracted with EtOAc (4×250 ml), and the organic extracts are combined, washed with water (50 ml), 5% aqueous LiCl solution (50 ml), dried (MgSO₄), filtered and evaporated. The residue is purified by silica gel (1 kg) chromatography eluting with 2:1 cyclohexane:EtOAc (6000 ml) followed by 1:1 cyclohexane:EtOAc (3000 ml). The fractions containing product are pooled and evaporated to give the title compound.

### Reference Intermediate 3 [1-ethyl-4-(tetrahydro-2H-pyran-4-ylamino)-1H-pyrazolo[3,4-b]pyridin-5-yl]methanol

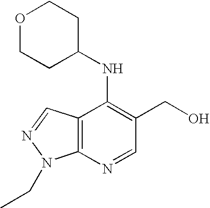

A solution of 1M diisobutylaluminium hydride in dichloromethane (80 ml) was added dropwise to a stirred solution of ethyl 1-ethyl-4-(tetrahydro-2H-pyran-4-ylamino)-1H-pyrazolo[3,4-b]pyridine-5-carboxylate (13.80 g) [e.g. see Intermediate 32 and/or Example 3 of WO 2004/024728 A2, and/or Intermediate 4 of WO 2005/058892 A1] in dichloromethane (75 ml) at 0° C. under a nitrogen atmosphere. The reaction mixture was maintained below 5° C. during the addition, and was then stirred at about 0° C. The reaction mixture was then quenched by addition of aqueous potassium sodium tartrate (10% solution), diluted with water (150 ml) and the organic phase separated. The aqueous phase was extracted with ethyl acetate (2×250 ml) and the combined organics were dried (magnesium sulphate) and evaporated. The residue was divided into three portions and each portion was purified by column chromatography on silica gel (100 g) eluting with a gradient of from 0 to 100% ethyl acetate in cyclohexane followed by from 0 to 20% methanol in ethyl acetate, and appropriate fractions were combined and evaporated, to give the title compound as a white solid (10.29 g). LCMS showed MH⁺=277; T_{RET}=1.81 min.

### Intermediate 4 [1,6-diethyl-4-(tetrahydro-2H-pyran-4-ylamino)-1H-pyrazolo[3,4-b]pyridin-5-yl]methanol

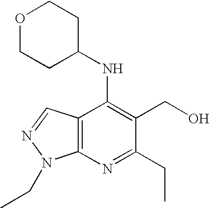

Intermediate 4 can be prepared, in an analogous manner to Reference Intermediate 3, from ethyl 1,6-diethyl-4-(tetrahydro-2H-pyran-4-ylamino)-1H-pyrazolo[3,4-b]pyridine-5-carboxylate (e.g. which can be as prepared in Intermediate 2).

### One Specific Synthesis of Intermediate 4:

Two reactions were done. The first reaction was as follows: To ethyl 1,6-diethyl-4-(tetrahydro-2H-pyran-4-ylamino)-1H-pyrazolo[3,4-b]pyridine-5-carboxylate (7.5 g) (e.g. which can be as prepared in Intermediate 2) in dichloromethane (75 ml) under nitrogen at 0° C. was added a solution of diisobutyaluminium hydride in toluene (1.5M, 43 ml), dropwise, keeping the temperature at 0° C. The addition took 9.5 mins. Stirring at 0° C. was continued for 30 mins; then the reaction was quenched with saturated aqueous sodium potassium tartrate solution (30 ml). There was much effervescence. The mixture gelled which made work-up more difficult. Ethyl acetate and water were added and the layers separated. The aqueous phase was extracted with more ethyl acetate and the combined organic extracts were washed with brine, dried and evaporated to give a white solid (6.13 g). The second duplicate reaction was generally the same (also using a 9.5 mins addition), and gave 6.59 g of product. The two batches of product from each reaction were each dissolved in chloroform and were filtered. The solutions from each duplicate reaction were combined and then evaporated to give the title compound as a white solid (12.57 g). LCMS showed MH⁺ 305; T_{RET}=1.84 min.

### Intermediate 4 (Alternative Synthesis B) [1,6-diethyl-4-(tetrahydro-2H-pyran-4-ylamino)-1H-pyrazolo[3,4-b]pyridin-5-yl]methanol

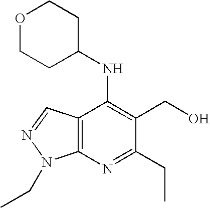

### Intermediate 4 (Alternative Synthesis B) Process Scheme

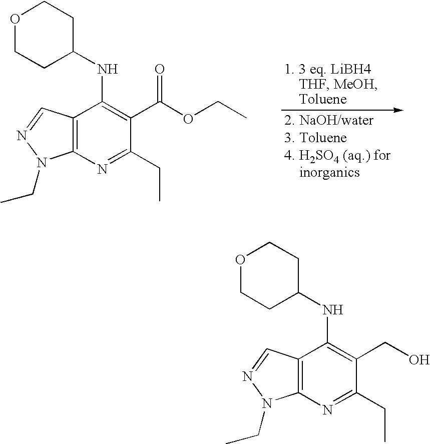

### Intermediate 4 (Alternative Synthesis B) Process Summary

Note: All or most of this process is carried out under a nitrogen atmosphere. All weights, volumes and equivalents are relative to ethyl 1,6-diethyl-4-(tetrahydro-2H-pyran-4-ylamino)-1H-pyrazolo[3,4-b]pyridine-5-carboxylate.

To a stirred solution of lithium borohydride (3 eq, 4.34 vol, 2M in THF) and toluene (4 vols) at 64-68° C., is added a solution of ethyl 1,6-diethyl-4-(tetrahydro-2H-pyran-4-ylamino)-1H-pyrazolo[3,4-b]pyridine-5-carboxylate (lwt) in THF (1 vol) and methanol (9 eq, 1.05 vol) over 2.5 hours. The reaction is monitored by HPLC and is typically complete 60 minutes after the addition. The reaction mixture is then cooled down to 20±3° C. and water (3 vol) is added dropwise over 20 mins followed by 10.8M NaOH (6.0 vol) (dropwise over 10 mins), ensuring the temperature remains below 40° C. The temperature of the reaction is then adjusted to 40±3° C. and stirred vigorously for at least 90 minutes. The organic layer is separated and the aqueous layer is transferred to a Schott bottle. Water is added to the organic layer (2.5 vols) followed by NaOH (10.8M, 2 vols), the biphase is stirred vigorously at 40° C. for at least 30 minutes. The organic layer is again separated and the organic solution is concentrated in vacuo to 3 vol, and the product precipitates as yellow solid. Toluene (3 vol) is then added and the slurry is reconcentrated in vacuo to 3 vol. The suspension is then cooled to 10±3° C. and aged for at least 30 min. The solid is then filtered and washed with toluene (3 vol). The product is then dried in vacuo at 45° C. to constant temperature.

### Intermediate 4 (Alternative Synthesis B) Procedure (Can be Run in 1.0 L or 10.0-L Equipment)

Note: All or most of this process is carried out under a nitrogen atmosphere. All weights, volumes and equivalents are relative to ethyl 1,6-diethyl-4-(tetrahydro-2H-pyran-4-ylamino)-1H-pyrazolo[3,4-b]pyridine-5-carboxylate.

1. Purge vessel with nitrogen.
2. Charge toluene (4 vols) to the vessel, mark 3 volumes during addition.
3. Charge LiBH₄ (2M in THF, 4.34 vols) to vessel.
4. Heat contents to 64-68° C.
5. Charge a Schott bottle with ethyl 1,6-diethyl-4-(tetrahydro-2H-pyran-4-ylamino)-1H-pyrazolo[3,4-b]pyridine-5-carboxylate (1 wt), add THF (1 vol) and MeOH (1.05 vols) and stir to dissolve contents.
6. Add the solution from step 5 to the LiBH₄ solution via peristaltic pump fitted with silicon tubing over 2.5 hours. Maintain Tᵢₙₜ at 64-68° C.
7. Stir the mixture for at 50-70 minutes at 64-68° C.
8. Sample the reaction and check for completeness by generic HPLC (typically complete at 60 minutes). The reaction can be held at 20° C. overnight at this point. Do not heat reaction beyond 70 minutes; start to cool at 70 minutes.
9. Cool contents of vessel to 20±3° C., add water (3 vols) to the vessel via peristaltic pump fitted with silicon tubing over at least 20 minutes. Ensure Tᵢₙₜ<40° C.
10. Adjust contents to 20±3° C. and add 10.8M sodium hydroxide (6.0 vols) to the vessel via peristaltic pump fitted with silicon tubing over 10 minutes, stir vigorously.
11. Adjust contents to 40±3° C. and stir for at least 90 minutes then allow layers to separate (separation<3 minutes).
12. Remove bottom aqueous layer from vessel. The organic layer can be held at this point at 20° C. overnight.
13. Add water (2.5 vols) and NaOH (10.8M, 2 vols) to the organic layer, stir biphase for at least 30 minutes at 40° C. and then allow layers to separate.
14. Remove bottom aqueous layer from vessel.
15. Concentrate the vessel contents to 3 vols using vacuum distillation.
16. Add toluene to the vessel (3 vols) and reconcentrate the contents to 3 vol via vacuum distillation.
17. Cool the contents to 10±3° C. and stir for at least 30 minutes. The batch can be held overnight at this point.
18. Filter off the product and wash the cake with toluene (1×3 vol) before sucking the cake dry (filtration times are both less than 2 minutes).
19. Transfer product to a vacuum oven and dry to constant temperature at 45-50° C. under vacuum (drying complete in less than 24 hours).
20. A yellow solid is obtained of [1,6-diethyl-4-(tetrahydro-2H-pyran-4-ylamino)-1H-pyrazolo[3,4-b]pyridin-5-yl]methanol.
21. For dispposal of aqueous layer, aqueous layer ex-step 12 and 14 can be charged to a vessel and the contents adjusted to 10±3° C. The stirred solution is acidified using 5M H₂SO₄ (11 vols) which is added over approx 1 hour (caution, exothermic). Check pH<3.

### Reference Intermediate 5 5-(chloromethyl)-1-ethyl-N-(tetrahydro-2H-pyran-4-yl)-1H-pyrazolo[3,4-b]pyridin-4-amine

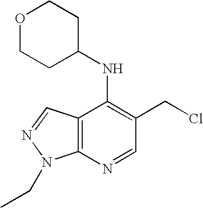

[1-ethyl-4-(tetrahydro-2H-pyran-4-ylamino)-1H-pyrazolo[3,4-b]pyridin-5-yl]methanol (80 mg) (e.g. which can be as prepared in Reference Intermediate 3) was treated with thionyl chloride (1 ml), heated at 80° C. for 1 h and then allowed to cool. The orange solution was evaporated to dryness and the residue azeotroped with toluene (2×5 ml) to give the title compound as a brown foam. LCMS of the compound in methanol showed MH⁺ 291, T_{RET}=1.90 min, which is consistent with the 5-(chloromethyl)-pyrazolo[3,4-b]pyridine derivative reacting with the methanol during LCMS to give the corresponding 5-(methoxymethyl)-pyrazolo[3,4-b]pyridine derivative.

### Intermediate 6 5-(chloromethyl)-1,6-diethyl-N-(tetrahydro-2H-pyran-4-yl)-1H-pyrazolo[3,4-b]pyridin-4-amine

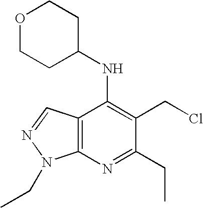

Intermediate 6 can be prepared, in an analogous manner to Reference Intermediate 5, from [1,6-diethyl-4-(tetrahydro-2H-pyran-4-ylamino)-1H-pyrazolo[3,4-b]pyridin-5-yl]methanol (e.g. which can be as prepared in Intermediate 4).

### One Specific Synthesis of Intermediate 6:

[1,6-diethyl-4-(tetrahydro-2H-pyran-4-ylamino)-1H-pyrazolo[3,4-b]pyridin-5-yl]methanol (0.120 g) (e.g. which can be Intermediate 4) is treated with thionyl chloride (1.5 ml), and the solution is heated at 80° C. for 1.5 h and then allowed to cool. The reaction mixture is concentrated to dryness and then azeotroped with toluene (5 ml) to leave the title compound.

### Reference Intermediate 7 5-(azidomethyl)-1-ethyl-N-(tetrahydro-2H-pyran-4-yl)-1H-pyrazolo[3,4-b]pyridin-4-amine

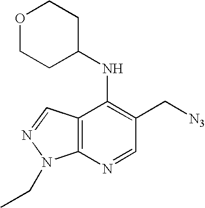

[1-ethyl-4-(tetrahydro-2H-pyran-4-ylamino)-1H-pyrazolo[3,4-b]pyridin-5-yl]methanol (0.552 g) (e.g. which can be as prepared in Reference Intermediate 3) was treated with thionyl chloride (2 ml, excess) and the solution was heated at 80° C. for 2 h and then allowed to cool. The thionyl chloride was evaporated off and the resulting solid was azeotroped with toluene (2×5 ml) to leave a yellow solid.

A solution of this solid, presumed to be 5-(chloromethyl)-1-ethyl-N-(tetrahydro-2H-pyran-4-yl)-1H-pyrazolo[3,4-b]pyridin-4-amine, (50 mg) in anhydrous dimethylsulphoxide (0.20 ml) was treated with lithium azide (9 mg) and the solution stirred at room temperature for 20 h. A further portion of lithium azide (15 mg) was then added, and, after a further day of stirring at room temperature, water (0.25 ml) was added. The solution was extracted with dichloromethane (2×5 ml) and the combined organic extracts were passed through a hydrophobic frit (6 ml) then blown to dryness to leave a clear colourless gum. This was dissolved in dichloromethane (0.5 ml) and applied to an SPE cartridge (silica; 1 g). The cartridge was eluted with 50% ethyl acetate in cyclohexane and fractions containing the desired material were combined and blown to dryness to give the title compound as a clear colourless gum (10 mg). LCMS showed MH⁺=302; T_{RET}=2.06 min.

### Intermediate 8 5-(azidomethyl)-1,6-diethyl-N-(tetrahydro-2H-pyran-4-yl)-1H-pyrazolo[3,4-b]pyridin-4-amine

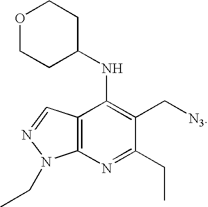

It is thought that Intermediate 8 can be prepared, in an analogous manner to Reference Intermediate 7, from 5-(chloromethyl)-1,6-diethyl-N-(tetrahydro-2H-pyran-4-yl)-1H-pyrazolo[3,4-b]pyridin-4-amine (e.g. which can be as prepared in Intermediate 6).

### One Specific Synthesis of Intermediate 8:

To [1,6-diethyl-4-(tetrahydro-2H-pyran-4-ylamino)-1H-pyrazolo[3,4-b]pyridin-5-yl]methanol (12.57 g) (e.g. which can be as prepared in Intermediate 4) was added thionyl chloride (51 ml, 83.6 g) and the mixture was heated to 80° C. There was effervescence during the addition, and the reaction mixture turned yellow, and then red on heating. After 5 h excess thionyl chloride was removed on an evaporator and the wine-red residue was azeotroped with toluene (75 ml) to leave a residue containing 5-(chloromethyl)-1,6-diethyl-N-(tetrahydro-2H-pyran-4-yl)-1H-pyrazolo[3,4-b]pyridin-4-amine.

This residue was dissolved in DMF (100 ml) and treated with sodium azide (4.0 g) and the mixture was stirred at room temperature overnight. Water was added, followed by 2M sodium hydroxide solution to adjust from a pH of about 3.5 to a pH of about 8. Ethyl acetate was added and the layers separated. The aqueous phase was extracted with more ethyl acetate. Then the combined organics were washed with water, aqueous lithium chloride solution and brine, and were dried and evaporated to give an orange-brown oil which was subject to high vacuum. This was purified by column chromatography using 400 ml of 7734 silica gel eluting with a 3:2 mixture of cyclohexane:ethyl acetate, and fractions containing a mixture of two products (as measured by TLC) were collected and reduced to give the title compound as a golden oil which started to crystallise under high vacuum (9.74 g). LCMS showed MH⁺=330; T_{RET}=2.30 min.

### Reference Intermediate 9 5-(aminomethyl)-1-ethyl-N-(tetrahydro-2H-pyran-4-yl)-1H-pyrazolo[3,4-b]pyridin-4-amine

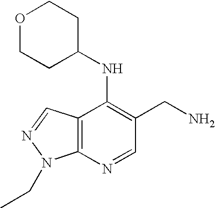

A solution of 5-(azidomethyl)-1-ethyl-N-(tetrahydro-2H-pyran-4-yl)-1H-pyrazolo[3,4-b]pyridin-4-amine (0.351 g) (e.g. which can be as prepared in Reference Intermediate 7) in ethanol (30 ml) was added to 5% palladium on carbon (wet) (0.050 g) and the mixture was stirred at room temperature for 20 hours under an atmosphere of hydrogen. The mixture was filtered first through a glass fibre filter and then through a plug of celite, which was then washed with ethanol (about 50 ml). The combined filtrate and washings were evaporated to dryness to give the title compound as a dirty green gum (0.318 g). LCMS showed MH⁺=276; T_{RET}=1.63 min.

### Intermediate 10 5-(aminomethyl)-1,6-diethyl-N-(tetrahydro-2H-pyran-4-yl)-1H-pyrazolo[3,4-b]pyridin-4-amine

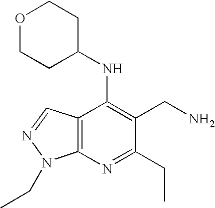

It is thought that Intermediate 10 can be prepared, in an analogous manner to Reference Intermediate 9, from 5-(azidomethyl)-1,6-diethyl-N-(tetrahydro-2H-pyran-4-yl)-1H-pyrazolo[3,4-b]pyridin-4-amine (e.g. which can be as prepared in Intermediate 8).

### One Specific Synthesis of Intermediate 10:

To 10% by weight palladium on carbon (0.4 g) was added ethanol (10 ml) followed by 5-(azidomethyl)-1,6-diethyl-N-(tetrahydro-2H-pyran-4-yl)-1H-pyrazolo[3,4-b]pyridin-4-amine (4 g) (e.g. which can be as prepared in Intermediate 8) in ethanol (90 ml) in a hydrogenation flask. The mixture was then hydrogenated at room temperature and atmospheric pressure with stirring overnight. Then, the palladium on carbon catalyst was filtered off twice to give a solution. The solvent was removed to give the title compound as a grey solid (2.59 g). LCMS showed MH⁺=304; T_{RET}=1.63 min. LCMS showed MH⁺=304; T_{RET}=1.63 min.

### Intermediate 11 5-(aminomethyl)-1,6-diethyl-N-(tetrahydro-2H-pyran-4-yl)-1H-pyrazolo[3,4-b]pyridin-4-amine hydrochloride

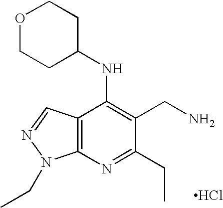

### Intermediate 11 Process Scheme

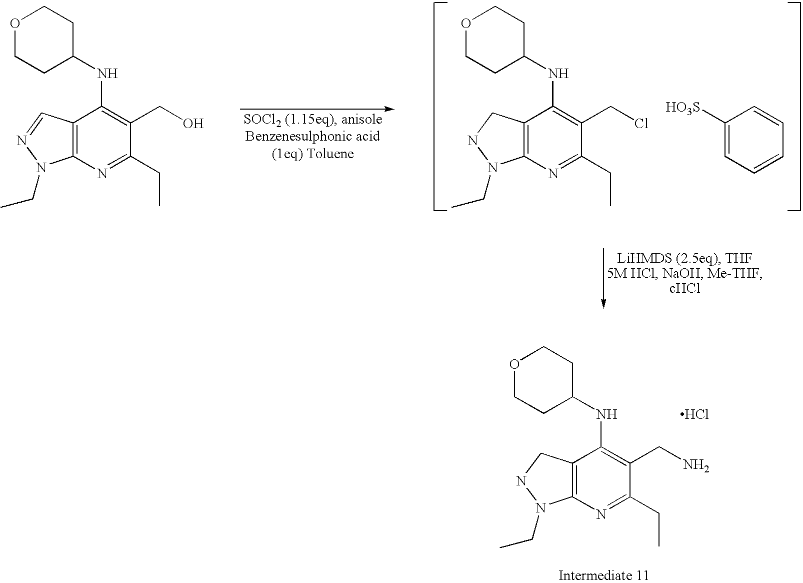

### Intermediate 11 Process Summary

Note: All or most of this process is carried out under a nitrogen atmosphere.

[1,6-diethyl-4-(tetrahydro-2H-pyran-4-ylamino)-1H-pyrazolo[3,4-b]pyridin-5-yl]methanol (e.g. which can be as prepared in Intermediate 4, e.g. Alternative Synthesis B thereof) is suspended in anisole and treated with solid benzensulfonic acid. This suspension is aged at room temperature for 30 minutes. Thionyl chloride is added at about 20 degrees C. over 20 minutes, and stirred for 20 minutes. The reaction is then sampled for HPLC. The resulting solution of 5-(chloromethyl)-1,6-diethyl-N-(tetrahydro-2H-pyran-4-yl)-1H-pyrazolo[3,4-b]pyridin-4-amine benzenesulfonate is treated with toluene and then placed under medium vacuum (100 mbar) to remove hydrogen chloride and sulfur dioxide, then the vacuum is increased (50 mbar) to azeotrope off excess thionyl chloride and toluene.

This solution of 5-(chloromethyl)-1,6-diethyl-N-(tetrahydro-2H-pyran-4-yl)-1H-pyrazolo[3,4-b]pyridin-4-amine benzenesulfonate is added to a solution of lithium hexamethyldisilazide (LiHMDS) in tetrahydrofuran at 35-40 degrees C. over about an hour and the reaction is usually complete after a further 10 minutes.

The mixture is cooled to 10 degrees C. and hydrochloric acid (5M) is added. The phases are separated and the lower aqueous layer is transferred back into the vessel, and the organic phase is discarded. Methyl-THF (methyl-tetrahydrofuran) is added and then the biphasic mixture is adjusted to pH>13 with sodium hydroxide solution (10M). The layers are separated and the lower aqueous layer is back extracted with further methyl-THF. The combined organic phase is washed with brine.

The amount of the desired 5-(aminomethyl)-1,6-diethyl-N-(tetrahydro-2H-pyran-4-yl)-1H-pyrazolo[3,4-b]pyridin-4-amine is quantified via yieldaliser, and an appropriate amount of concentrated hydrochloric acid is added at 55-60 degrees C. The suspension is held for 2 hours at about 50 degrees C., and then cooled to 10 degrees C. over 3 hours and held at this temperature for at least 3 hours.

The slurry is filtered and washed with methyl-THF and dried at 60° C. to constant weight or batch temperature, to give 5-(aminomethyl)-1,6-diethyl-N-(tetrahydro-2H-pyran-4-yl)-1H-pyrazolo[3,4-b]pyridin-4-amine hydrochloride (Intermediate 11).

### Intermediate 11 Detailed Procedure: (Can be Run in 1.0/10 L Equipment)

Note: All or most of this process is carried out under a nitrogen atmosphere.

1. [1,6-diethyl-4-(tetrahydro-2H-pyran-4-ylamino)-1H-pyrazolo[3,4-b]pyridin-5-yl]methanol (1 wt) is suspended in anisole (6 vol) at 20-25° C.
2. Benzenesulfonic acid (1 equivalent, 0.52 wt) is added.
3. The temperature is adjusted to 20-25° C. and the reaction is aged for 30 minutes.
4. Thionyl chloride (1.15 equivalents, 0.45 wt, 0.275 vol) is added over about 20 minutes at 20-25° C.
5. Anisole (0.25 vol) is used to wash the line.
6. Sample after 20 minutes at 20-25° C. Reaction is judged complete when <4% of [1,6-diethyl-4-(tetrahydro-2H-pyran-4-ylamino)-1H-pyrazolo[3,4-b]pyridin-5-yl]methanol remains when compared to the 5-(methoxymethyl)-pyrazolo[3,4-b]pyridine derivative derived from a methanol quench of 5-(chloromethyl)-1,6-diethyl-N-(tetrahydro-2H-pyran-4-yl)-1H-pyrazolo[3,4-b]pyridin-4-amine benzenesulfonate.
7. Vessel fill volume is noted, and toluene (2 vol) added.
8. The mixture is heated to a jacket temperature of 40 degrees C. and held under moderate vacuum (100 mbar) for 1 hour with agitation.
9. Vacuum is increased to about 50 mbar and contents are distilled to marked volume above (about 7.5 vol). The Contents should not be heated above 50 degrees C. (Distillation time around 80 minutes, p=45-50 mbar, Tᵢₙₜ=20-48° C.)
10. Reaction mixture is cooled to 20-25° C. and can be held at this point.
11. This mixture (ex-step 10) is added to lithium hexamethyldisilazide in THF (1.26M in THF, 6.51 vol, 5.73 wt) over 60-70 minutes at 35-40° C.
12. Sample after a further 10 minutes at 35-40° C. Reaction is judged complete if <4% of the 5-(methoxymethyl)-pyrazolo[3,4-b]pyridine derivative is seen relative to 5-(aminomethyl)-1,6-diethyl-N-(tetrahydro-2H-pyran-4-yl)-1H-pyrazolo[3,4-b]pyridin-4-amine (after quench into methanol) by HPLC.
13. Mixture is cooled to 10° C. and hydrochloric acid (5M, 3.6 vol) is added over 20 minutes. The phases are stirred vigorously for 10 minutes.
14. The lower aqueous layer is retained, and the upper anisole layer discarded. The batch can be held at 20-25° C. at this point.
15. The brown aqueous layer is treated with methyl-tetrahydrofuran (8 vol).
16. Sodium hydroxide solution (ca 1.8 vol, 10M) is added to the biphasic mixture to give a resulting aqueous layer with pH>13. The temperature is raised to 30° C. and the mixture is stirred vigorously for 20 minutes (separation time 5 minutes in 10 L CLR equipment).
17. The layers are separated and the lower aqueous layer (yellow) is backwashed with methyl-tetrahydrofuran (2 vol). The aqueous layer is sampled and analysed for residual 5-(aminomethyl)-1,6-diethyl-N-(tetrahydro-2H-pyran-4-yl)-1H-pyrazolo[3,4-b]pyridin-4-amine prior to disposal.
18. Brine (15% w/w NaCl, 2 vol) is added to the combined brown organic layers the biphase is vigorously stirred at 30° C. and separated. The lower aqueous layer is removed.
19. The volume of the organic layer is measured and a sample taken. The yieldaliser system is used to determine the absolute amount of 5-(aminomethyl)-1,6-diethyl-N-(tetrahydro-2H-pyran-4-yl)-1H-pyrazolo[3,4-b]pyridin-4-amine present.
20. The reaction mixture is heated to 55-60° C. and treated with concentrated hydrochloric acid [1.03 equivalents relative to the 5-(aminomethyl)-1,6-diethyl-N-(tetrahydro-2H-pyran-4-yl)-1H-pyrazolo[3,4-b]pyridin-4-amine present, about 0.24-0.30 vol].
21. The mixture is aged at 50-55° C. for 2 hours and then cooled to 5-10° C. over 3 hours.
22. The mixture is aged at 5-10° C. for at least 3 hours.
23. Mixture is filtered and washed with methyl-THF (2×2 vol). The mother liquors are kept until a dried weight and yield of product is obtained. (Filtration times all less then 90 seconds on a scale of about 700 g.)
24. The off white solid cake is dried in a vacuum oven at 60° C. to constant temperature.

### Intermediate 12: Tetrahydro-2H-pyran-4-amine hydrochloride, which is 4-aminotetrahydro-2H-pyran hydrochloride

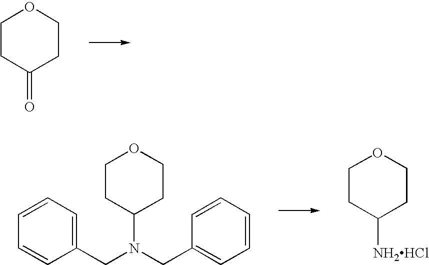

### Step 1: N,N-dibenzyltetrahydro-2H-pyran-4-amine

Dibenzylamine (34.5 g) and acetic acid (6.7 ml) are added to a stirred solution of tetrahydro-4H-pyran-4-one (16.4 g, e.g. commercially available from Aldrich) in dichloromethane (260 ml) at 0° C. to 5° C. After 2.5 h at 0° C. to 5° C., sodium triacetoxyborohydride (38.9 g) is added portionwise, and the mixture is allowed to warm to room temperature. After stirring at room temperature overnight, the reaction mixture is washed successively with 2M-sodium hydroxide (200 ml and 50 ml), water (2×50 ml) and brine (50 ml), then dried and evaporated. The residue (which may be an oil) is stirred with methanol (50 ml) at 4° C. for 30 min to give the product (e.g. as a solid).

### Step 2: Tetrahydro-2H-pyran-4-amine hydrochloride

N,N-dibenzyltetrahydro-2H-pyran-4-amine (20.5 g) is dissolved in ethanol (210 ml) and hydrogenated over 10% palladium on carbon catalyst (4 g) at 100 psi for 72 h at room temperature. The reaction mixture is filtered and the filtrate is adjusted to pH 1 with 2M-hydrogen chloride in diethyl ether. Evaporation of solvents gives a residue (which may be a solid) which is triturated with diethyl ether to give the product (which may be a solid).

### EXAMPLES

### Example 1

### Synthesis A

### N-{[1,6-diethyl-4-(tetrahydro-2H-pyran-4-ylamino)-1H-pyrazolo[3,4-b]pyridin-5-yl]methyl}-3-methyl-5-isoxazolecarboxamide

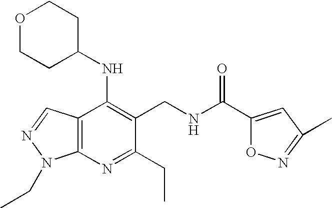

An example of a specific synthesis of Example 1 is as follows:

3-Methyl-5-isoxazolecarboxylic acid (92 mg) was suspended in dry dichloromethane (2 ml) and treated at 20° C. with oxalyl chloride (0.064 ml) and diethyl formamide (1 drop). Effervescence occurred over ca. 10 mins, and after 40 mins stirring at room temperature the solution was added dropwise to a solution of 5-(aminomethyl)-1,6-diethyl-N-(tetrahydro-2H-pyran-4-yl)-1H-pyrazolo[3,4-b]pyridin-4-amine (198 mg) (e.g. which can be as prepared in Intermediate 10) in anhydrous acetonitrile (4 ml). The mixture was treated with DIPEA (0.128 ml) and stirred at room temperature under nitrogen for 15 h. Dichloromethane (50 ml) was added to the reaction mixture and the solution was washed with dilute aqueous sodium chloride solution (2×50 ml). The organic phase was separated using a hydrophobic frit (70 ml) and loaded directly onto an SPE cartridge (10 g, aminopropyl) which had been pre-washed with methanol. The cartridge was eluted with methanol (×2) and the fractions collected and blown down/evaporated to dryness. The residual gum was dissolved in dichloromethane and purified by SPE cartridge (10 g, silica) on a Flashmaster 2 eluting with a gradient of 0-100% ethyl acetate in cyclohexane over 40 mins. Two fractions (from two chromatographic peaks) were collected and evaporated separately, and each was purified by mass directed autoprep HPLC. Relevant fractions from each were evaporated to dryness. The two residual gums (125 mg and 48 mg respectively) were combined and purified by SPE cartridge (2 g, aminopropyl) which had been pre-washed with methanol. Elution with methanol (×2), collection of methanol, and evaporation to dryness gave the title compound as a white foam (173 mg). LCMS showed MH⁺=413; T_{RET}=2.20 min.

Note: 3-Methyl-5-isoxazolecarboxylic acid is thought to be commercially available from one or more of the following suppliers: Chemical Block Stock Library, Chemical Block Building Blocks, Fluorochem, Scientific Exchange, Aurora Screening Library, Oakwood Products Catalog, Ambinter Stock Screening Collection, TimTec Building Blocks and Reagents, TimTec Overseas Stock, Enamine Building Blocks, Enamine Screening Library, Interchim Intermediates, AsInEx Express Gold Collection, and/or MicroChemistry Building Blocks. See near the start of the “Intermediates and Examples” section hereinabove, for the addresses of some of these suppliers.

### Example 1

### Synthesis B

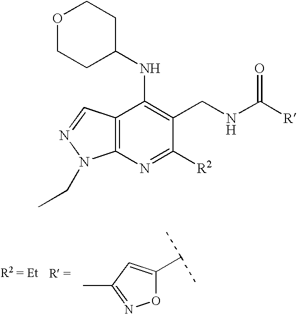

The carboxylic acid R′COOH

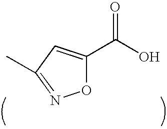

(0.12 mmol) is treated with a solution of HATU (0.12 mmol) in DMF (0.25 ml), and DIPEA (0.052 ml, ca. 0.3 mmol) is added. The solution is shaken for 10 mins, and is treated with a solution of 5-(aminomethyl)-1,6-diethyl-N-(tetrahydro-2H-pyran-4-yl)-1H-pyrazolo[3,4-b]pyridin-4-amine (0.1 mmol, ca. 30.3 mg) (e.g. which can be as prepared in Intermediate 10) in DMF (0.2 ml). The resulting solution is shaken for 10 mins and left to stand for 18 h (e.g. at room temperature), and then the DMF is removed in a Genevac vacuum centrifuge. The residue is dissolved in chloroform (0.3 ml), is applied to an SPE cartridge (1 g, aminopropyl) which has been pre-washed with chloroform (6 ml), and is eluted sequentially with chloroform (3 ml) and 10% methanol in ethyl acetate (3 ml). Fractions containing the desired product are concentrated in vacuo in a Genevac vacuum centrifuge, and where necessary the residue is purified by mass directed autoprep HPLC (e.g. acetonitrile/water). Where necessary, the compound is dissolved in chloroform, and is further purified by loading onto a SPE cartridge (0.5 g, aminopropyl) which has been prewashed with chloroform, eluting with 10% methanol in ethyl acetate.

### Example 1

### Synthesis C

### N-{[1,6-diethyl-4-(tetrahydro-2H-pyran-4-ylamino)-1H-pyrazolo[3,4-b]pyridin-5-yl]methyl}-3-methyl-5-isoxazolecarboxamide

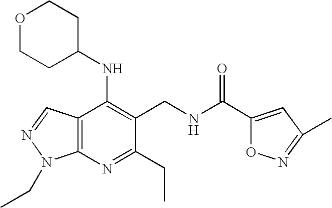

3-Methyl-5-isoxazolecarboxylic acid (6.59 g, 0.052 mol, 1.0 equivalent), O-(benzotriazol-1-yl)-N,N,N′N¹-tetramethyluronium hexafluorophosphate (HBTU) (20.88 g, 1.08 equivalents) and diisopropylethylamine (18 ml, 2 equivalents) in dimethyl formamide (DMF, 314 ml, 20 volumes) were stirred under nitrogen for 10 minutes. 5-(aminomethyl)-1,6-diethyl-N-(tetrahydro-2H-pyran-4-yl)-1H-pyrazolo[3,4-b]pyridin-4-amine (15.7 g, 0.052 mol, 1.0 equivalent) (e.g. which can be as prepared in Intermediate 10) was added to the mixture with extra DMF (20 ml) to wash it into the flask. The reaction mixture was left to stir for three (3) hours, and then left under nitrogen overnight. The reaction mixture was partitioned between saturated sodium hydrogen carbonate solution (3 L) and dichloromethane (800 mL). The aqueous layer was extracted again with dichloromethane (800 mL). The combined organic layers were backwashed with water (2×500 mL), dried over MgSO₄ (50 g), filtered, reduced in vacuo, and cooled to give a crude brown oil (about 30 g) which appeared to include DMF.

This crude brown oil was slurried in 1:0.98:0.2 DCM: EtOAc: MeOH, whereupon the crude product precipitated, was filtered and the filtered solid was washed with diethyl ether (100 ml) to give a damp mass (12 g). The reduced liquors (the reduced slurry liquids remaining after filtration and the diethyl ether washings) were purified by column chromatography (silica, 800 ml), using 1:0.98:0.2 DCM: EtOAc: MeOH (4 L) as eluent, to give a further batch of product (4 g).

Both batches of product were combined (appeared impure by ¹H NMR), and were purified by column chromatography (silica, 800 ml), using 3 to 4% MeOH in DCM (3 L) as eluent, to give 13 g of material which still appeared impure (by TLC).

This resulting impure solid (13 g) was slurried in 1:0.96:0.4 of DCM: EtOAc: MeOH and the solid remaining was separated by filtration and dried, to give the title compound N-{[1,6-diethyl-4-(tetrahydro-2H-pyran-4-ylamino)-1H-pyrazolo[3,4-b]pyridin-5-yl]methyl}-3-methyl-5-isoxazolecarboxamide as an off-white solid (9 g).

The reduced liquors (the reduced slurry liquids) were purified by column chromatography (silica, 400 ml) using 1:0.96:0.4 DCM: EtOAc: MeOH (2 L) as eluent, giving, after reduction in vacuo, a further batch of product.

The two batches of product were combined to give 10.714 g in total of the title compound N-{[1,6-diethyl-4-(tetrahydro-2H-pyran-4-ylamino)-1H-pyrazolo[3,4-b]pyridin-5-yl]methyl}-3-methyl-5-isoxazolecarboxamide as an off-white solid (more than about 95% purity as measured by NMR in CDCl₃ solvent (not the NMR given below)).

¹H NMR (500 MHz in d₆-DMSO, δ (delta) ppm): 9.38 (br t, 1H), 8.04 (s, 1H), 7.00 (s, 1H), 6.64 (d, 1H), 4.52 (d, 2H), 4.33 (q, 2H), 4.12 (br s, 1H), 3.88 (d, 2H), 3.57 (t, 2H), 2.95 (q, 2H), 2.29 (s, 3H), 1.93 (d, 2H), 1.54 (q, 2H), 1.35 (t, 3H), 1.24 (t, 3H). Plus trace other peaks: possibly solvent.

### PHARMACEUTICAL COMPOSITION EXAMPLES

The following are examples of pharmaceutical compositions (formulations) suitable for external topical administration (e.g. topical administration to skin).

### Composition Example 1

### Ointment D

The following pharmaceutical composition (formulation) is an ointment suitable for external topical administration (e.g. topical administration to skin), and comprises 2% w/w of the compound of the invention N-{[1,6-diethyl-4-(tetrahydro-2H-pyran-4-ylamino)-1H-pyrazolo[3,4-b]pyridin-5-yl]methyl}-3-methyl-5-isoxazolecarboxamide (i.e. the “free base” form):

Product: Ointment D
Batch Size: 20 g

| | Concentration in | | |
|---|---|---|---|
| | composition | Theoretical | Actual |
| Ingredient | (% w/w) | Amount (g) | Amount (g) |
| white petrolatum | 73 | 14.6 | 14.69 |
| decamethyl- | 25 | 5 | 5.01 |
| cyclopentasiloxane | | | |
| (ST-Cyclomethicone | | | |
| 5-NF ™) | | | |
| compound of the | 2 | 0.4 | 0.4006 |
| invention (i.e. “free | | | |
| base” form) | | | |
| TOTAL | | 20 | 20.1006 |

### Preparation Procedure:

Approximately the following procedure is used to prepare Ointment D:

The white petrolatum (a solid at room temperature, and e.g. which can optionally be high melting point white petrolatum such as Penreco Ultima White™ grade white petrolatum) and the decamethyl-cyclopentasiloxane (a liquid at room temperature) are heated together, in a beaker in a hot water bath, to a temperature of approximately 60-65° C., to melt the white petrolatum.

The drug substance (the compound of the invention, which, for example, can be as prepared in Example 1, Synthesis C) is added slowly into the melted oil phase and is stirred with a spatula and dispersed completely. The mixture is homogenised under high shear conditions (using setting #5, a high setting, on an Ultra-turrax T25 homogenizer), using the small homogenizer shaft, for 10 minutes.

The formulation is allowed to cool to room temperature (e.g. about 17 to about 22° C.) while stirring. The ointment formulation is filled into a 30 mL transparent container.

### Reference Composition Example 2

### Placebo Ointment AP

The following pharmaceutical composition (formulation) is an ointment suitable for external topical administration (e.g. topical administration to skin), but it does not include any compound of the invention. Thus it can be used as a comparator placebo ointment, when testing (e.g. in “In Vivo Assay A” herein) a corresponding ointment containing the compound of the invention such as Composition Example 1 (Ointment D).

Product: Placebo Ointment AP
Batch Size: 100 g

| | Concentration | | Actual |
|---|---|---|---|
| | in composition | Theoretical | Amount |
| Ingredient | (% w/w) | Amount (g) | (g) |
| white petrolatum | 75 | 75 | 75.61 |
| decamethyl-cyclopentasiloxane | 25 | 25 | 25.00 |
| (ST-Cyclomethicone 5-NF ™) | | | |
| TOTAL | | 100 | 100.61 |

### Preparation Procedure:

Approximately the following procedure is used to prepare Placebo Ointment AP:

The white petrolatum (which can optionally be high melting point white petrolatum such as Penreco Ultima White™ grade white petrolatum) and the decamethyl-cyclopentasiloxane are heated together, in a small beaker in a hot water bath, to a temperature of approximately 60-65° C.

The mixture is homogenised under high shear conditions (using setting #5, a high setting, on an Ultra-turrax T25 homogenizer), for approximately 10 minutes.

The formulation is allowed to cool and is stirred until room temperature (e.g. about 17 to about 22° C.) is reached. The ointment formulation is packed into a 125 mL transparent plastic container.

### Composition Example 3

### Ointment D2

The following pharmaceutical composition (formulation) is an ointment suitable for external topical administration (e.g. topical administration to skin), and comprises 2% w/w of the compound of the invention N-{[1,6-diethyl-4-(tetrahydro-2H-pyran-4-ylamino)-1H-pyrazolo[3,4-b]pyridin-5-yl]methyl}-3-methyl-5-isoxazolecarboxamide (i.e. the “free base” form):

Product: Ointment D2
Batch Size: 20 g

| | Concentration | | |
|---|---|---|---|
| | in | | Actual |
| | composition | Theoretical | Amount |
| Ingredient | (% w/w) | Amount (g) | (g) |
| white petrolatum | 73 | 14.6 | 14.66 |
| decamethyl- | 25 | 5 | 5.09 |
| cyclopentasiloxane | | | |
| (ST-Cyclomethicone 5-NF ™) | | | |
| compound of the invention | 2 | 0.4 | 0.4012 |
| (i.e. “free base” form) | | | |
| TOTAL | | 20 | 20.1512 |

### Preparation Procedure:

Substantially the same preparation procedure as in Composition Example 1 (Ointment D) is used to prepare Ointment D2.

### Reference Composition Example 4

### Placebo Ointment AP2

The following pharmaceutical composition (formulation) is an ointment suitable for external topical administration (e.g. topical administration to skin), but it does not include any compound of the invention. Thus it can be used as a comparator placebo ointment, when testing (e.g. in “In Vivo Assay A” herein) a corresponding ointment containing the compound of the invention such as Composition Example 3 (Ointment D2).

Product: Placebo Ointment AP2
Batch Size: 100 g

| | Concentration | | Actual |
|---|---|---|---|
| | in composition | Theoretical | Amount |
| Ingredient | (% w/w) | Amount (g) | (g) |
| white petrolatum | 75 | 75 | 75.52 |
| decamethyl- | 25 | 25 | 25.03 |
| cyclopentasiloxane | | | |
| (ST-Cyclomethicone 5-NF ™) | | | |
| TOTAL | | 100 | 100.55 |

### Preparation Procedure:

Substantially the same preparation procedure as in Reference Composition Example 2 (Placebo Ointment AP) is used to prepare Placebo Ointment AP2.

### Composition Example 5

### Water-in-Oil Cream Cr-D

The following pharmaceutical composition (formulation) is believed to be a water-in-oil cream emulsion, for external topical administration (e.g. topical administration to skin), and comprises propylene glycol and 2% w/w of the compound of the invention N-{[1,6-diethyl-4-(tetrahydro-2H-pyran-4-ylamino)-1H-pyrazolo[3,4-b]pyridin-5-yl]methyl}-3-methyl-5-isoxazolecarboxamide (i.e. the “free base” form):

Product: Water-in-oil cream Cr-D
Batch Size: 20 g

| | Concentration | | Actual |
|---|---|---|---|
| | in composition | Theoretical | Amount |
| Ingredient | (% w/w) | Amount (g) | (g) |
| white petrolatum | 40 | 8 | 8.05 |
| mineral oil | 10 | 2 | 2.00 |
| steareth-2 = polyoxyl | 8 | 1.6 | 1.62 |
| 2 stearyl ether | | | |
| (Volpo s-2 ™) (surfactant) | | | |
| Aqueous phase | | | |
| propylene glycol | 20 | 4 | 4.01 |
| purified water | 20 | 4 | 4.01 |
| compound of the invention | 2 | 0.4 | 0.4001 |
| (i.e. “free base” form) | | | |
| TOTAL | | 20 | 20.0901 |

### Preparation Procedure:

Approximately the following procedure is used to prepare Water-in-oil cream Cr-D:

The white petrolatum (which can optionally be high melting point white petrolatum such as Penreco Ultima White™ grade white petrolatum), the mineral oil and the steareth-2 (Volpo s-2™) are heated together, via a hot water bath, to a temperature of approximately 60-65° C. to form an oily phase.

In a separate container, the drug substance (the compound of the invention, which, for example, can be as prepared in Example 1, Synthesis C) is dispersed in water and sonicated for approximately 10 minutes. Propylene glycol is added to this aqueous phase and the mixture is sonicated for another 10 minutes. The drug substance is at least partly in suspension.

The aqueous phase is heated to approximately the same temperature as the oily phase (e.g. is heated to approximately 60-65° C.), and then the aqueous phase is added slowly to the oily phase while homogenizing the mixture under high shear conditions (using setting #5, a high setting, e.g. on an Ultra-turrax T25 homogenizer), for approximately 10 minutes.

After homogenization, the formulation is allowed to cool to room temperature (e.g. about 17 to about 22° C.) with constant mixing with a spatula. The cream formulation is packed into a 20 mL scintillation vial.

### Reference Composition Example 6

### Placebo Water-in-Oil Cream Cr-AP

The following pharmaceutical composition (formulation) is believed to be a water-in-oil cream emulsion, for external topical administration, but it does not include any compound of the invention. Thus it can be used as a comparator placebo cream, when testing (e.g. in “In Vivo Assay A” herein) a corresponding cream containing the compound of the invention such as Composition Example 5 (Water-in-oil cream Cr-D).

Product: Placebo Water-in-oil cream Cr-AP
Batch Size: 100 g

| | Concentration | | Actual |
|---|---|---|---|
| | in composition | Theoretical | Amount |
| Ingredient | (% w/w) | Amount (g) | (g) |
| white petrolatum | 42 | 42 | 42.98 |
| mineral oil | 10 | 10 | 10.06 |
| steareth-2 = polyoxyl 2 stearyl | 8 | 8 | 8.04 |
| ether (Volpo s-2 ™) (surfactant) | | | |
| Aqueous phase | | | |
| propylene glycol | 20 | 20 | 20.06 |
| purified water | 20 | 20 | 20.03 |
| TOTAL | | 100 | 101.17 |

### Preparation Procedure:

Approximately the following procedure is used to prepare Placebo Water-in-oil cream Cr-AP:

The white petrolatum (which can optionally be high melting point white petrolatum such as Penreco Ultima White™ grade white petrolatum), the mineral oil and the steareth-2 (Volpo s-2™) are heated together, via a hot water bath, to a temperature of approximately 60-65° C. to form an oily phase. In another beaker, the aqueous phase (propylene glycol and water) is heated to a temperature of approximately 60-65° C.

The aqueous phase is added slowly to the oily phase while homogenizing the mixture at low speed, and then the speed is increased to high shear conditions (using setting #5, a high setting, e.g. on an Ultra-turrax T25 homogenizer). The mixture is homogenized, while being kept hot via a hot water bath, for approximately 10 minutes.

The formulation is then allowed to cool and stirred until room temperature (e.g. about 17 to about 22° C.) is reached. The cream formulation is packed into a 125 mL transparent plastic container.

### Composition Example 7

### Water-in-Oil Cream Cr-D2

The following pharmaceutical composition (formulation) is believed to be a water-in-oil cream emulsion, for external topical administration (e.g. topical administration to skin), and comprises propylene glycol and 2% w/w of the compound of the invention N-{[1,6-diethyl-4-(tetrahydro-2H-pyran-4-ylamino)-1H-pyrazolo[3,4-b]pyridin-5-yl]methyl}-3-methyl-5-isoxazolecarboxamide (i.e. the “free base” form):

Product: Water-in-oil cream Cr-D2
Batch Size: 20 g

| | Concentration | | Actual |
|---|---|---|---|
| | in composition | Theoretical | Amount |
| Ingredient | (% w/w) | Amount (g) | (g) |
| white petrolatum | 40 | 8 | 8.01 |
| mineral oil | 10 | 2 | 2.02 |
| steareth-2 = polyoxyl 2 stearyl | 8 | 1.6 | 1.64 |
| ether (Volpo s-2 ™) | | | |
| (surfactant) | | | |
| Aqueous phase | | | |
| propylene glycol | 20 | 4 | 4.03 |
| purified water | 20 | 4 | 4.07 |
| compound of the invention | 2 | 0.4 | 0.4004 |
| (i.e. “free base” form) | | | |
| TOTAL | | 20 | 20.1704 |

### Preparation Procedure:

Substantially the same preparation procedure as in Composition Example 5 (Water-in-oil cream Cr-D) is used to prepare Water-in-oil cream Cr-D2, except that the cream formulation is packed into a 30 mL transparent container.

### Reference Composition Example 8

### Placebo Water-in-Oil Cream Cr-AP2

The following pharmaceutical composition (formulation) is believed to be a water-in-oil cream emulsion, for external topical administration, but it does not include any compound of the invention. Thus it can be used as a comparator placebo cream, when testing (e.g. in “In Vivo Assay A” herein) a corresponding cream containing the compound of the invention such as Composition Example 7 (Water-in-oil cream Cr-D2).

Product: Placebo Water-in-oil cream Cr-AP2
Batch Size: 100 g

| | Concentration | | Actual |
|---|---|---|---|
| | in composition | Theoretical | Amount |
| Ingredient | (% w/w) | Amount (g) | (g) |
| white petrolatum | 42 | 42 | 42.71 |
| mineral oil | 10 | 10 | 10.15 |
| steareth-2 = polyoxyl 2 stearyl | 8 | 8 | 8.01 |
| ether (Volpo s-2 ™) (surfactant) | | | |
| Aqueous phase | | | |
| propylene glycol | 20 | 20 | 20.00 |
| purified water | 20 | 20 | 20.02 |
| TOTAL | | 100 | 101.89 |

### Preparation Procedure:

Substantially the same preparation procedure as in Reference Composition Example 6 (Placebo Water-in-oil cream Cr-AP) is used to prepare Placebo Water-in-oil cream Cr-AP2.

1. N-{[1,6-diethyl-4-(tetrahydro-2H-pyran-4-ylamino)-1H-pyrazolo[3,4-b]pyridin-5-yl]methyl}-3-methyl-5-isoxazolecarboxamide 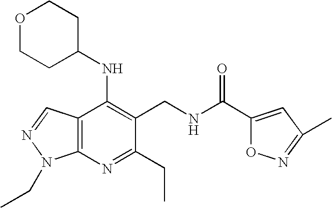
or a salt thereof.

2. A compound as claimed in claim 1 or a pharmaceutically acceptable salt thereof.

3. N-{[1,6-diethyl-4-(tetrahydro-2H-pyran-4-ylamino)-1H-pyrazolo[3,4-b]pyridin-5-yl]methyl}-3-methyl-5-isoxazolecarboxamide

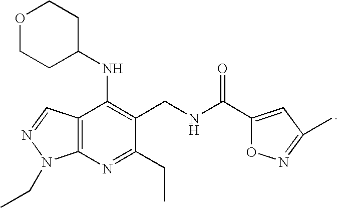

4. A compound or salt as claimed in claim 2, wherein the pharmaceutically acceptable salt comprises a pharmaceutically acceptable acid addition salt.

5. A compound or salt as claimed in claim 2, wherein the pharmaceutically acceptable salt is a pharmaceutically acceptable acid addition salt.

6. (canceled)

7. A compound or salt as claimed in claim 4, wherein the pharmaceutically acceptable acid addition salt of the N-{[1,6-diethyl-4-(tetrahydro-2H-pyran-4-ylamino)-1H-pyrazolo[3,4-b]pyridin-5-yl]methyl}-3-methyl-5-isoxazolecarboxamide comprises a hydrobromide, hydrochloride, sulfate, nitrate, phosphate, p-toluenesulfonate, benzenesulfonate, methanesulfonate, ethanesulfonate, or naphthalenesulfonate salt thereof.

8. (canceled)

9. A process for preparing a compound of formula (I) or a salt thereof: 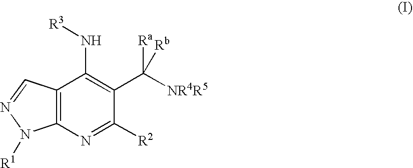
wherein R¹ is ethyl; R² is ethyl;
R³ is a heterocyclic group of sub-formula (bb) which is not substituted on a ring carbon: 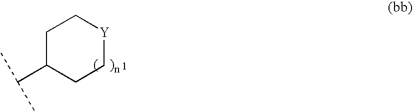
in which n1 is 1; and in which Y is O;
and wherein:
R^{a} is a hydrogen atom (H); R^{b} is a hydrogen atom (H);
R⁴ is a hydrogen atom (H); R⁵ is —C(O)—(CH₂)ₙ—Ar; wherein n is 0;
and Ar has the sub-formula (z) which is sub-formula (z9): 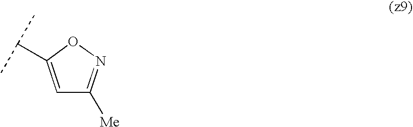
which process comprises:
(A) reacting an amine of formula (II) or a salt thereof 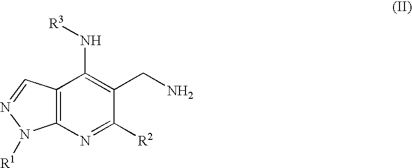
with a compound Ar—C(O)—X¹, wherein X¹ is a leaving group substitutable by the NH₂ amine moiety of the compound of formula (II);
and optionally converting the compound of formula (I) into a salt thereof;
or (B), in a process for preparing a pharmaceutically acceptable salt of a compound of formula (I), converting the compound of formula (I) or a salt thereof into a desired pharmaceutically acceptable salt of the compound of formula (I).

10. A process as claimed in claim 9, which is a process for preparing a compound of formula (I) or a pharmaceutically acceptable salt thereof, and wherein:
in process (A), in the compound Ar—C(O)—X¹,
X¹ is a chlorine atom (Cl),
or X¹ is 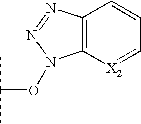
wherein X₂ is CH or N;
or X¹ is O—C(NHR^{C1})═NR^{C2} wherein R^{C1} and R^{C2} are independently C₁₋₄alkyl, cyclohexyl or 3-dimethylaminopropyl;
and/or
in process (B), the process comprises conversion of the compound of formula (I) or a salt thereof into a desired pharmaceutically acceptable acid addition salt of the compound of formula (I).

11. (canceled)

12. A pharmaceutical composition comprising a compound of formula (I), as defined in claim 1, or a pharmaceutically acceptable salt thereof, and one or more pharmaceutically acceptable carriers and/or excipients.

13. (canceled)

14. A pharmaceutical composition as claimed in claim 12, which is suitable for external topical administration to a mammal such as a human.

15. (canceled)

16. A pharmaceutical composition as claimed in claim 14, which is for the treatment and/or prophylaxis of atopic dermatitis in a mammal such as a human, by external topical administration to the mammal.

17.-20. (canceled)

21. An pharmaceutical composition as claimed in claim 16 being an ointment and comprising:
the compound as defined in claim 1 or the pharmaceutically acceptable salt thereof present at 0.5% to 10% w/w;
white petrolatum present at 45% to 99.5% w/w (i.e. by weight of the composition); and
a silicone oil present at 5% to 50% w/w (measured as the total silicone oil content, by weight of the composition)).

22. An pharmaceutical composition as claimed in claim 20, being an ointment and comprising:
the compound as defined in claim 1 or the pharmaceutically acceptable salt thereof present at 1% to 10% w/w;
white petrolatum present at 45% to 99.5% w/w; and
decamethyl-cyclopentasiloxane present at 5% to 50% w/w.

23-29. (canceled)
